(19) United States
(12) Patent Application Publication
(10) Pub. No.: US 2026/0028396 A1
(43) Pub. Date: Jan. 29, 2026

(54) ANTI-MYOSTATIN ANTIBODIES AND METHODS

(71) Applicants: AstralBio, Inc., Boston, MA (US); iBio, Inc., San Diego, CA (US)

(72) Inventors: Patrick Crutcher, Boston, MA (US); Justin DiMartino, Boston, MA (US); Cory Schwartz, San Diego, CA (US); Cody Allen Moore, San Diego, CA (US); Alexander Tomoaki Taguchi, San Diego, CA (US); Martin Brenner, San Diego, CA (US)

(21) Appl. No.: 19/336,730

(22) Filed: Sep. 23, 2025

Related U.S. Application Data
(60) Provisional application No. 63/674,644, filed on Jul. 23, 2024.
(60) Provisional application No. 63/697,692, filed on Sep. 23, 2024.

(51) Int. Cl. C07K 16/22; A61K 39/00

(52) U.S. Cl. CPC C07K 16/22; A61K 2039/505

(57) ABSTRACT

Provided herein are anti-myostatin antibodies which bind myostatin. The anti-myostatin antibodies of the disclosure are useful for the treatment of muscle dystrophies by blocking myostatin signaling. Also provided herein are methods of use of the anti-myostatin antibodies.

### CROSS-REFERENCE TO RELATED APPLICATIONS

This application claims priority to U.S. Provisional Application Ser. No. 63/697,692, filed Sep. 23, 2024 and U.S. Provisional Application Ser. No. 63/764,644, filed Feb. 28, 2025, and, the entire contents of which are incorporated herein by reference.

### TECHNICAL FIELD OF THE INVENTION

The present invention relates to anti-myostatin antibodies and methods of making and using the same.

### INCORPORATION-BY-REFERENCE OF MATERIALS FILED ON COMPACT DISC

The application contains a Sequence Listing which has been submitted electronically in .XML format and is hereby incorporated by reference in its entirety. Said .XML copy, created on Sep. 22, 2025, is named “IBIO1043.xml” and is 239,458 bytes in size. The sequence listing contained in this .XML file is part of the specification and is hereby incorporated by reference herein in its entirety.

### BACKGROUND OF THE INVENTION

Without limiting the scope of the invention, its background is described in connection with anti-myostatin antibodies.

One such patent is U.S. Pat. No. 11,155,611, issued to Scholar Rock and U.S. Patent Publication Nos. 20180344844, 20170333558, and 20170198032, which are directed to compositions and methods for making and using anti-myostatin antibodies. These inventors are said to teach antibodies, or antigen-binding fragments thereof, that specifically bind proMyostatin and/or latent Myostatin, and methods and uses thereof for treating metabolic diseases.

Another such patent application is U.S. Patent Publication No. 20190002548, filed by Ruike and Kuramochi, entitled “Anti-Myostatin Antibodies and Methods of Use”. These applicants are said to teach an anti-myostatin antibody that binds to mature myostatin, and uptake of the antibody into cells is enhanced when complexed with the antigen. The invention also provides isolated nucleic acids encoding an anti-myostatin antibody, host cells comprising a nucleic acid encoding the antibody, and a method of producing the antibody. The anti-myostatin are said to be used as a medicament for treating a muscle wasting disease, increasing mass of muscle tissue, and increasing strength of muscle tissue.

Yet another such patent application is U.S. Patent Publication No. 20130209489, filed by Han, et al., entitled “Antibodies that Bind Myostatin, Compositions and Methods”. These applicants are said to teach anti-myostatin antibodies, nucleic acids encoding them, and methods of making and using them, wherein the neutralizing antibodies recognizing the conformational epitope near position 21 to 31 and position 50 to 60.

Despite these advances, a need remains for novel anti-myostatin antibodies with enhanced targeted activity and use in treating muscle wasting disease(s), increasing the mass of muscle tissue, and increasing the strength of muscle tissue.

### SUMMARY OF THE INVENTION

As embodied and broadly described herein, an aspect of the present disclosure relates to an anti-myostatin antibody or antigen binding domain or fragment thereof, wherein the antibody or antigen binding domain thereof comprises: a heavy chain variable domain (VH) complementarity determining region (CDR) 1, VH CDR2, and VH CDR3 comprising the amino acid sequence of any one of the following SEQ ID NOS: 3, 4, 5; 13, 14, 15; 23, 24, 25; 33, 34, 35; 43, 44, 45; 53, 54, 55; 63, 64, 65; 73, 74, 75; 83, 84, 85; 93, 94, 95; 103, 104, 105; 113, 114, 115; 123, 124, 125; 133, 134, 135; 143, 144, 145; 153, 154, 155; 163, 164, 165; 173, 174, 175; 183, 184, 185; 193, 194, 195; 203, 204, 205; 213, 214, 215; 223, 224, 225; 233, 234, 235; 243, 244, 245; or 253, 254, 255, respectively; and a light chain variable domain (VL) CDR1, VL CDR2, and VL CDR3 comprising the amino acid sequence of any one of the following SEQ ID NOS: 6, 7, 8; 16, 17, 18; 26, 27, 28; 36, 37, 38; 46, 47, 48; 56, 57, 58; 66, 67, 68; 76, 77, 78; 86, 87, 88; 96, 97, 98; 106, 107, 108; 116, 117, 118; 126, 127, 128; 136, 137, 138; 146, 147, 148; 156, 157, 158; 166, 167, 168; 176, 177, 178; 186, 187, 188; 196, 197, 198; 206, 207, 208; 216, 217, 218; 226, 227, 228; 236, 237, 238; 246, 247, 248; or 256, 257, 258, respectively; or a heavy chain amino acid sequence having at least 95, 96, 97, 98, 99, or 100% sequence identity to any one of the following SEQ ID NOS: 1, 11, 21, 31, 41, 51, 61, 71, 81, 91, 101, 111, 121, 131, 141, 151, 161, 171, 181, 191, 201, 211, 221, 231, 241, or 251, respectively; and a light chain amino acid sequence having at least 95, 96, 97, 98, 99, or 100% sequence identity to any one of the following SEQ ID NOS: 2, 12, 22, 32, 42, 52, 62, 72, 82, 92, 102, 112, 122, 132, 142, 152, 162, 172, 182, 192, 202, 212, 222, 232, 242, or 252, respectively. In one aspect, antibody or antigen binding domain comprises: a VH comprising the amino acid sequence of any one of the following SEQ ID NOS: 1, 11, 21, 31, 41, 51, 61, 71, 81, 91, 101, 111, 121, 131, 141, 151, 161, 171, 181, 191, 201, 211, 221, 231, 241, or 251, and a VL comprising the amino acid sequence of any one of the following SEQ ID NOS: 2, 12, 22, 32, 42, 52, 62, 72, 82, 92, 102, 112, 122, 132, 142, 152, 162, 172, 182, 192, 202, 212, 222, 232, 242, or 252. In another aspect, the antibody is a monoclonal antibody. In another aspect, the antibody is a full-length antibody. In another aspect, the antibody is an antibody domain or single chain antigen binding domain of the heavy chain selected from SEQ ID NOS: 9, 19, 29, 39, 49, 59, 69, 79, 89, 99, 109, 119, 129, 139, 149, 159, 169, 179, 189, 199, 209, 219, 229, 239, 249, or 259; and the light chain selected from SEQ ID NOS: 10, 20, 30, 40, 50, 60, 70, 80, 90, 100, 110, 120, 130, 140, 150, 160, 170, 180, 190, 200, 210, 220, 230, 240, 250, or 260. In another aspect, the antigen binding domain is fused to an Fc domain of any one of the following: human IgG1, human IgG2, human IgG3, and human IgG4. In another aspect, the antibody comprises an Fc domain having an amino acid sequence selected from SEQ ID NOS: 261, 263, or 265. In another aspect, the antibody or antigen binding domain heavy chain amino acid sequence is encoded by a nucleic acid having at least 90, 91, 92, 93, 94, 95, 96, 97, 98, 99, or 100% sequence identity to any one of the following SEQ ID NOS: 9, 19, 29, 39, 49, 59, 69, 79, 89, 99, 109, 119, 129, 139, 149, 159, 169, 179, 189, 199, 209, 219, 229, 239, 249, or 259; and the antibody or antigen binding domain light chain amino acid sequence is encoded by a nucleic acid having at least 90, 91, 92, 93, 94, 95, 96, 97, 98, 99, or 100% sequence identity to any one of the following SEQ ID NOS: 10, 20, 30, 40, 50, 60, 70, 80, 90, 100, 110, 120, 130, 140, 150, 160, 170, 180, 190, 200, 210, 220, 230, 240, 250, or 260.

As embodied and broadly described herein, an aspect of the present disclosure relates to a method of treating a muscle condition in a subject in need thereof, comprising administering to the subject a therapeutically effective amount of the antibody or antigen binding domain or fragment described hereinabove. In one aspect, the muscle condition is selected from myopathy, muscular atrophy, muscular dystrophy, and nerve injury, sarcopenia, cachexia, Parkinson's disease, osteoporosis, osteoarthritis, osteopenia, muscle injury, muscle wasting from disuse, immobilization, bed rest, injury, medical treatments, or surgical interventions. In another aspect, the muscle condition is associated with spinal muscular atrophy (SMA), amyotrophic lateral sclerosis (ALS), or myasthenia gravis. In another aspect, the nerve injury comprises partial denervation of neurons that innervate muscle, or impaired signaling between a motor neuron and a target muscle. In another aspect, the metabolic disease selected from the group consisting of type I diabetes, type II diabetes, obesity, metabolic syndrome/pre-diabetes, cardiovascular disease, non-alcoholic steatohepatitis (NASH), spinal cord injury (SCI), a hypo-metabolic state, double diabetes, Cushings disease, and an obesity syndrome. In another aspect, the antibody or antigen binding domain is provided in an amount sufficient to treat at least one of: treatment of a muscle wasting disease, increasing mass of muscle tissue, increasing strength of muscle tissue, enhance force generation, or prevent muscle loss, wherein the subject has, or is at risk of, at least one of: developing myopathy, muscle atrophy, or a metabolic disorder. In another aspect, the subject is human.

As embodied and broadly described herein, an aspect of the present disclosure relates to a method of treating a disease in a subject in need thereof, comprising administering to the subject a therapeutically effective amount of the antibody or antigen binding domain described hereinabove. In one aspect, the antibody or antigen binding domain is provided in an amount effective to cause one or more of the following in the subject: (a) an increase in mass and/or function of a muscle tissue in the subject; (b) an increase in the metabolic rate of the subject; (c) an increase in insulin sensitivity of the subject; (d) an increase in a level of brown adipose tissue in the subject; (e) an increase in a level of beige adipose tissue in the subject; (f) a decrease in a level of white adipose tissue in the subject; (g) a decrease in a level of visceral adipose tissue in the subject; (h) a decrease in ratio of adipose-to-muscle tissue in the subject; (i) an increase in glucose uptake by a brown adipose tissue, a beige adipose tissue, or a muscle tissue in the subject; (j) a decrease in glucose uptake by a white adipose tissue or a liver tissue; (k) a decrease in muscle catabolism of protein and/or muscle release of amino acids in the subject; (l) an increase in insulin dependent glycemic control in the subject; (m) a decrease in intramuscular fat infiltration in the subject; (n) an improvement in quality of life, as assessed by a standardized quality of life test; (o) prevention of muscle loss or atrophy in the subject; and/or, (p) prevention of developing a metabolic dysregulation associated with muscle dysfunction in the subject, wherein the subject is a human subject that benefits from reduced myostatin signaling. In another aspect, the subject is human.

As embodied and broadly described herein, an aspect of the present disclosure relates to a polynucleotide encoding an anti-myostatin antibody or antigen binding domain or fragment thereof comprising: a heavy chain amino acid sequence is encoded by a nucleic acid having at least 90, 91, 92, 93, 94, 95, 96, 97, 98, 99, or 100% sequence identity to any one of the following SEQ ID NOS: 9, 19, 29, 39, 49, 59, 69, 79, 89, 99, 109, 119, 129, 139, 149, 159, 169, 179, 189, 199, 209, 219, 229, 239, 249, or 259; and a light chain amino acid sequence is encoded by a nucleic acid having at least 90, 91, 92, 93, 94, 95, 96, 97, 98, 99, or 100% sequence identity to any one of the following SEQ ID NOS: 10, 20, 30, 40, 50, 60, 70, 80, 90, 100, 110, 120, 130, 140, 150, 160, 170, 180, 190, 200, 210, 220, 230, 240, 250, or 260. In one aspect, the antibody is a monoclonal, bispecific, multivalent, multi-specific, diabody, chimeric, scFv antibody, or domain thereof. In another aspect, the antibody binding domain is fused to an Fc domain of any one of the following: human IgG1, human IgG2, human IgG3, and human IgG4. In another aspect, the nucleic acid sequence is optimized for expression in a human, mouse, rat, hamster, bacterial, fungal, insect, or plant cell. In another aspect, the polynucleotide further comprising a vector comprising the polynucleotides claimed hereinabove. In another aspect, the polynucleotide of claim 19, further comprising a host cell comprising the vector claimed hereinabove.

As embodied and broadly described herein, an aspect of the present disclosure relates to a method of making an anti-myostatin antibody or binding domain or fragment thereof comprising expression in a host cell a polynucleotide that expresses: a heavy chain variable domain (VH) complementarity determining region (CDR) 1, VH CDR2, and VH CDR3 comprising the amino acid sequence of any one of the following SEQ ID NOS: 3, 4, 5; 13, 14, 15; 23, 24, 25; 33, 34, 35; 43, 44, 45; 53, 54, 55; 63, 64, 65; 73, 74, 75; 83, 84, 85; 93, 94, 95; 103, 104, 105; 113, 114, 115; 123, 124, 125; 133, 134, 135; 143, 144, 145; 153, 154, 155; 163, 164, 165; 173, 174, 175; 183, 184, 185; 193, 194, 195; 203, 204, 205; 213, 214, 215; 223, 224, 225; 233, 234, 235; 243, 244, 245; or 253, 254, 255, respectively; and a light chain variable domain (VL) CDR1, VL CDR2, and VL CDR3 comprising the amino acid sequence of any one of the following SEQ ID NOS: 6, 7, 8; 16, 17, 18; 26, 27, 28; 36, 37, 38; 46, 47, 48; 56, 57, 58; 66, 67, 68; 76, 77, 78; 86, 87, 88; 96, 97, 98; 106, 107, 108; 116, 117, 118; 126, 127, 128; 136, 137, 138; 146, 147, 148; 156, 157, 158; 166, 167, 168; 176, 177, 178; 186, 187, 188; 196, 197, 198; 206, 207, 208; 216, 217, 218; 226, 227, 228; 236, 237, 238; 246, 247, 248; or 256, 257, 258, respectively; or a heavy chain amino acid sequence is encoded by a nucleic acid having at least 90, 91, 92, 93, 94, 95, 96, 97, 98, 99, or 100% sequence identity to any one of the following SEQ ID NOS: 9, 19, 29, 39, 49, 59, 69, 79, 89, 99, 109, 119, 129, 139, 149, 159, 169, 179, 189, 199, 209, 219, 229, 239, 249, or 259; and a light chain amino acid sequence is encoded by a nucleic acid having at least 90, 91, 92, 93, 94, 95, 96, 97, 98, 99, or 100% sequence identity to any one of the following SEQ ID NOS: 10, 20, 30, 40, 50, 60, 70, 80, 90, 100, 110, 120, 130, 140, 150, 160, 170, 180, 190, 200, 210, 220, 230, 240, 250, or 260.

### BRIEF DESCRIPTION OF THE DRAWINGS

For a more complete understanding of the features and advantages of the present invention, reference is now made to the detailed description of the invention along with the accompanying figures and in which:

FIG. 1 shows SPR affinity measurements of binding to GDF8 or GDF11 for phage display-selected clones.

FIG. 2 shows SPR affinity measurements of binding to GDF8 or GDF11 for phage display and mammalian display-selected clones.

FIG. 3A shows reporter cell line to measure GDF8 antagonism. Antibodies expressed with Fc_1.

FIG. 3B shows reporter cell line to measure GDF11 antagonism. Antibodies expressed with Fc_1.

FIG. 4A shows myotube coverage as measure of myoblast differentiation. GDF8 antagonism. Antibodies made with Fc_1.

FIG. 4B shows myotube coverage as measure of myoblast differentiation. GDF11 antagonism. Antibodies made with Fc_1.

FIG. 5A shows a fusion index as measure of myoblast differentiation. GDF8 antagonism. Antibodies made with Fc_1.

FIG. 5B shows a fusion index as measure of myoblast differentiation. GDF11 antagonism. Antibodies made with Fc_1.

FIG. 6A shows the binding kinetics for antibody and Fc combinations against human FcRn via BLI.

FIG. 6B is an isoaffinity plot of antibody and Fc combinations for binding against human FcRn via BLI.

FIG. 7A shows myotube coverage as measure of myoblast differentiation.

FIG. 7B shows the IC50 (nM) for antibody and Fc combinations against GDF8 and GDF11 in myoblast differentiation, for both myotube coverage and fusion index.

FIG. 8A shows the measure of antibody size distribution by DLS, reported as PDI, after treatment under several stress conditions.

FIG. 8B shows the measure of antibody monomericity by HPLC-SEC, reported as percent monomer.

FIG. 9A shows a diet-induced obesity (DIO) mouse study design to test Ab_15 with semaglutide. FIG. 9B is a graph that shows the baseline corrected percent change in lean mass measured by DEXA scan.

FIG. 10A shows an Obese NHP study design to test Ab_15 Fc_3, and FIG. 10B is a graph that shows serum antibody concentration levels over time, after single I.V. dose at 2 levels. FIG. 10C is a table with the calculated linear elimination half-life, and FIG. 10D is a graph that shows a change in lean mass from baseline based on DEXA scan analysis.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It should be understood that, unless clearly indicated, in any method described or disclosed herein that includes more than one act, the order of the acts is not necessarily limited to the order in which the acts of the method are recited, but the disclosure encompasses exemplary embodiments in which the order of the acts is so limited.

### Use of Myostatin Inhibitors, Such as Anti-Myostatin Antibodies and Antigen Binding Domain (or Fragments) Thereof, for Treating Diseases/Disorders

The pharmaceutical compositions described herein are suitable for administration to human patients for the treatment or prevention of diseases and conditions where reduced myostatin signaling is desirable. Such diseases and conditions include, but are not limited to, e.g., muscle conditions or disorders, metabolic disorders, and diseases associated with impaired neurological signaling, e.g., spinal cord injury. Non-limiting examples of diseases or conditions for which the compositions and methods of the present invention may be useful are further described below.

Incretin-based therapies are becoming standard treatments for weight loss. However, up to 40% of the weight lost through these medications is attributed to reductions in lean muscle mass. Myostatin is produced by and acts on muscle cells to trigger muscle wasting. Homozygous loss of function mutations leads to significant muscle hypertrophy without obvious deleterious health effects. Expressed as homodimer and signals through activin receptors and Smad2/3 pathway. Beyond muscle, myostatin plays a role in the regulation of adipogenesis & leads to reduction in total body fat mass, visceral & intramuscular fat. Thus, the present invention in one aspect enhances the quality of weight loss by maintaining lean muscle mass during weight loss. By generating the novel antibodies herein, it was possible to highly selective binders with optimized target product profile for obesity by subcutaneous administration and having an extended half-life. For example, the half-life can be extended in non-human primate studies with a predicted 7, 8, 9, 10, 11, 12, 13, 14, 15, 16, 17, 18, 19, 20, 21, 22, 23, 24, 25, 26, 27, 28, 29, 30, 35, 40, or 45 days supports every 2 months to every 3 months dosing in humans.

As compared to present antibodies, the present invention provides not only high potency, but can also be provided subcutaneously in a low or minimal volume (e.g., 0.1, 0.2, 0.3, 0.4, 0.5, 0.6, 0.7, 0.9, 1, 2, 3, 4, 5, 10, 15, 20, 25, 30, 40, 50, 60, 70, 75, 80, 90, 100, 200, 300, 400, 500, 600, 700, 750, 800, 900, or 1,000 milliliters). Further, the present invention targets extended dosing frequency (e.g., every 4 weeks, every 6 weeks, every 8 weeks, every 10 weeks, every 12 weeks, or less frequently). Thus, the present invention overcomes the problem with existing anti-myostatin antibodies such as the combination treatment with trevogrumab and garetosmab mAb, bimagrumab, and/or apitegromab, GYM329″, “taldefgrobep alfa, which cannot be applied in a small/low volume subcutaneously, do not enable extended dosing frequency, and fail to provide the safety margin of the present invention.

### Muscle Conditions and Disorders

In some aspects, the methods of the present invention are suitable for treating or preventing muscle conditions and disorders. As used herein, the term “muscle condition” or “muscle disorder” refers to a disease, condition, or disorder, where the muscle does not function normally, or a disease, condition, or disorder, where the function of muscle is normal, but there is less force generated by the muscle due to a reduced amount of muscle available. A muscle condition or disorder may include, without limitation, a myopathy, muscular atrophy, a muscular dystrophy, etc. Such conditions may be caused by a defect or defects in a motor neuron, a genetic mutation, or an injury, such as a nerve injury.

In another aspect, the muscle condition is a myopathy. As used herein, the term “myopathy” refers to a muscular condition characterized by impaired muscle structure or function, typically resulting in muscular weakness; a muscular condition characterized by normal muscle structure but impaired or abnormal neuronal input, which in turn affects muscle function; or inflammatory myopathies and/or autoimmune myopathies, e.g., myasthenia gravis.

Myopathies for treatment with the antibodies or antigen binding domain or fragments can also include muscular conditions that are neuromuscular or musculoskeletal in nature. The myopathy can be an inherited myopathy such as, e.g., dystrophies, myotonias, congenital myopathies (nemaline myopathy, multi/minicore myopathy, or centronuclear myopathy), mitochondrial myopathies, familial periodic myopathies, inflammatory myopathies and metabolic myopathies (glycogen storage diseases and lipid storage disorder). In some aspects, the myopathy is an acquired myopathy, such as, e.g., external substance induced myopathy (drug-induced myopathy and glucocorticoid myopathy, alcoholic myopathy, and myopathy due to other toxic agents), myositis (dermatomyositis, polymositis and inclusion body myositis), myositis ossificans, rhabdomyolysis, and myoglobinurias, and disuse atrophy. In some aspects, the myopathy is disuse atrophy, which may be caused by prolonged disuse of muscles, leading to deterioration of normal muscle function, such as e.g., atrophy resulting from hospitalization, bone fracture(s), or by nerve injury. In some aspect the myopathy is related to a disease or disorder such as amyotrophic lateral sclerosis (ALS), spinal muscular atrophy (SMA), cachexia syndromes due to renal failure, cardiac conditions, long-COVID, and/or cancer. In some aspect, the myopathy is related to ageing, sarcopenia, or paraspinal muscle atrophy (PMA).

In some aspects, the myopathy is a primary myopathy. In one aspect, a primary myopathy comprises disuse atrophy resulting from, e.g., hip fracture, elective joint replacement, critical care myopathy, spinal cord injury or stroke. In some embodiments, the myopathy is a genetic muscle weakness associated with, for example, a muscular dystrophy.

In some aspects, the myopathy is a secondary myopathy, in which muscle loss or dysfunction is secondary to a disease pathology, such as denervation or cachexia, denervation associated with monitor neuron dysfunction. In some aspects, motor neuron dysfunction is due to genetic mutation(s) that affect motor neurons, such as, e.g., amyotrophic lateral sclerosis (ALS) and spinal muscular atrophy (SMA). In some embodiments, the secondary myopathy is a cachexia associated with renal failure, long-COVID, a cardiac condition, cancer or aging. In some aspects, the secondary myopathy is caused by a nerve injury, such as, nerve injury sustained during a medical procedure, such as surgeries. Detrimental effects of such injury to the function of a target tissue (e.g., target muscle) may be effectively treated by administration of the anti-myostatin antibodies or antigen binding domain(s) described herein. For example, such administration may prevent and/or alleviate myopathy, and/or facilitate recovery.

The antibodies described herein are valuable for the treatment, prevention, and/or mitigation of diseases or disorders linked to myostatin activity. In particular, these antibodies are beneficial in treating conditions that can be improved by enhancing muscle strength, power, mass, or function in an individual, or by positively influencing metabolic processes (e.g., carbohydrate, lipid, and protein metabolism) through the inhibition of myostatin activity. Examples of diseases, disorders, and conditions treatable with the anti-myostatin antibodies disclosed herein include, but are not limited to, sarcopenia, cachexia (whether idiopathic or secondary to conditions such as cancer, chronic kidney disease, or chronic obstructive pulmonary disease), muscle injuries, muscle wasting, and muscle atrophy, such as those arising from or associated with disuse, immobilization, bed rest, injury, medical treatments, or surgical interventions (e.g., hip fractures, hip or knee replacements) or the necessity for mechanical ventilation. The anti-myostatin antibodies may also be employed in the treatment, prevention, or mitigation of diseases including, but not limited to, cancer, obesity, diabetes, arthritis, multiple sclerosis, muscular dystrophy, amyotrophic lateral sclerosis, Parkinson's disease, osteoporosis, osteoarthritis, osteopenia, and metabolic syndromes (such as diabetes, obesity, nutritional disorders, organ atrophy, chronic obstructive pulmonary disease, and anorexia).

In some aspects, the antibodies, or antigen binding domain or fragments thereof can be used in methods for treating or preventing muscle conditions and disorders, including paraspinal muscle atrophy, nerve injury-dependent muscle atrophy, postoperative nerve injury-dependent muscle atrophy, a lumbar spine surgery, a lumbar spine procedure (e.g., a lumbar fusion procedure, a lumbar nonfusion procedure, a posterior lumbar fusion procedure, an anterior lumbar fusion procedure, a minimally invasive (MIS) posterior lumbar decompression procedure, a minimally invasive (MIS) posterior lumbar fusion procedure, a non-MIS equivalent procedure), etc.

In one aspect, the methods of treatment with the myostatin inhibitors, e.g., antibodies, or antigen binding domain thereof, described herein results in at least 1%, 2%, 3%, 4%, 5%, 6%, 7%, 8%, 9%, 10%, 12%, 15%, 18%, 20%, or 25% decrease in muscle atrophy. In one embodiment, methods of treatment with the myostatin inhibitors, e.g., antibodies, or antigen binding domain thereof, described herein results in preventing at least a 1%, 2%, 3%, 4%, 5%, 6%, 7%, 8%, 9%, 10%, 12%, 15%, 18%, 20%, or 25% of the muscle atrophy.

As used herein, a “subject” may be a mammalian subject. Mammalian subjects include, humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

### Antibodies

As used herein, the term “antibody” refers to an intact antibody or a binding domain or fragment thereof that binds specifically to a target antigen. Binding domains are produced by recombinant DNA techniques, or by enzymatic or chemical cleavage of intact antibodies. Binding domains or fragments include Fab, Fab′, F(ab′)₂, Fv, and single-chain variable domain (scFv) antibodies. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay). The term “antibody” is used in the broadest sense, and specifically covers monoclonal antibodies (including full-length antibodies or other bivalent, Fc-region containing antibodies such as bivalent scFv Fc-fusion antibodies), polyclonal antibodies, multispecific antibodies (e.g., bispecific antibodies), and antibody fragments (e.g., Fab, Fab′, F(ab′)₂, Fv, scFv) so long as they exhibit the desired biological activity. Antibodies (Abs) and immunoglobulins (Igs) are glycoproteins having the same structural characteristics. The present invention includes monoclonal antibodies (and binding domains thereof) that are completely recombinant, in other words, where the complementarity determining regions (CDRs) are genetically spliced into a human antibody backbone, often referred to as veneering an antibody. Thus, in certain aspects, the monoclonal antibody is a fully synthesized antibody. In certain embodiments, the monoclonal antibodies (and binding fragments thereof) can be made in bacterial or eukaryotic cells, including plant cells.

As used herein, the terms “antigen binding domain” or “antibody fragment” refer to a portion of a full-length antibody, generally the antigen-binding or variable region, and include Fab, Fab′, F(ab′)₂, Fv, and scFv fragments. Papain digestion of antibodies produces two identical antigen-binding fragments, called the Fab fragment, each with a single antigen-binding site, and a residual “Fc” fragment, so-called for its ability to crystallize readily. Pepsin treatment yields an F(ab′)₂ fragment that has two antigen-binding fragments which are capable of cross-linking antigen, and a residual other fragment (which is termed pFc′). As used herein, “functional fragment” with respect to antibodies, refers to Fv, F(ab) and F(ab′)₂ fragments.

As used herein, the “Fv” fragment is the minimum antibody fragment that contains a complete antigen recognition and binding site. This region consists of a dimer of one heavy and one light chain variable domain in a tight, non-covalent association (V_{H}-V_{L} dimer). It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

Native antibodies and immunoglobulins are usually heterotetrameric glycoproteins of about 150,000 daltons, composed of two identical light (L) chains and two identical heavy (H) chains. Each light chain is linked to a heavy chain by at least one covalent disulfide bond, however, the number of disulfide linkages varies between the heavy chains of different immunoglobulin isotypes. Each heavy and light chain also has regularly spaced intrachain disulfide bridges. Each heavy chain has at one end a variable domain (V_{H}) followed by the constant domains. Each light chain has a variable domain at one end (V_{L}) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaccous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the terms “antibody mutant” or “antibody variant” refer to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90, 92, 93, 94, 95, 96, 97, 98, or 99% sequence identity.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another: 1) Alanine (A) and Glycine (G); 2) Aspartic acid (D) and Glutamic acid (E); 3) Asparagine (N) and Glutamine (Q); 4) Arginine (R) and Lysine (K); 5) Isoleucine (I), Leucine (L), Methionine (M), and Valine (V); 6) Phenylalanine (F), Tyrosine (Y), and Tryptophan (W); 7) Serine (S) and Threonine (T); and/or 8) Cysteine (C) and Methionine (M).

As used herein, the term “host cell” refers to any eukaryotic or prokaryotic cell (e.g., mammalian cells, avian cells, amphibian cells, plant cells, fish cells, and insect cells), whether located in vitro or in vivo.

The polynucleotide (nucleic acid) sequences encoding the antibody and antigen binding domains or fragments disclosed herein may be codon optimized using techniques well known in the art. Codon optimization refers to the fact that different cells differ in their usage of particular codons (such as in a mammalian cell, e.g., a human cell). The codon bias corresponds to the relative abundance of particular tRNAs in the cell type. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC: aspartic acid can be encoded by GAT or GAC; cysteine can be encoded by TOT or TGC: alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, Biochemistry, 2023, 10th Edition, MacMillan Learning, which is incorporated herein by reference in its entirety). By altering the codons in the polynucleotide sequence to match the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Generally, the polynucleotide sequence is optimized for expression in CHO or human cells, such as one having at least 70, 75, 80, 90, 91, 92, 93, 94, 95, 96, 97, 98, 99, or 100% sequence identity to its corresponding wild-type or originating polynucleotide sequence. In one example, a codon optimized coding sequence has at least 80, 90, 91, 92, 93, 94, 95, 96, 97, 98, 99, or 100% sequence identity to its corresponding wild-type or originating sequence. In another embodiment, a eukaryotic cell codon optimized nucleic acid sequence encodes a polynucleotide having at least 80, 90, 91, 92, 93, 94, 95, 96, 97, 98, 99, or 100% sequence identity to its corresponding wild-type or originating protein. In another embodiment, a variety of clones comprising functionally equivalent nucleic acids may be routinely generated, such as nucleic acids which differ in sequence, but which encode the same protein sequence.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used herein, the term “variable” in the context of the variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342:877), or both, that is Chothia plus Kabat. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al.) The constant domains are not involved directly in binding an antibody to its cognate antigen but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain. Depending on the amino acid sequences of the constant domain of their heavy chains, “immunoglobulins” can be assigned to different classes. There are at least five (5) major classes of immunoglobulins: IgA, IgD, IgE, IgG, and IgM, and several of these may be further divided into subclasses (isotypes), e.g., IgG-1, IgG-2, IgG-3, and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed invention may be made by the hybridoma method first described by Kohler and Milstein, Nature 256, 495 (1975), relevant portions incorporated herein by reference.

The antibodies in this invention were the result of a mutagenesis library and in vitro selection process. The starting antibody was originally from an immunization.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted therefore, while the antibodies' affinity for the myostatin is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains, domains or fragments thereof (such as Fab, Fab′, F(ab′)₂, Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e., rodent, chicken) CDRs or CDR sequences for the corresponding sequences of a human antibody, see, e.g., U.S. Pat. No. 5,225,539. In some instances, Fv framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of, at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

The presently disclosed and claimed invention further includes the use of fully human monoclonal antibodies cross-reactive against other mammalian myostatins. Fully human antibodies essentially relate to antibody molecules in which the entire sequence of both the light chain and the heavy chain, including the CDRs, arise from human genes. Such antibodies are termed “human antibodies” or “fully human antibodies” herein. Human monoclonal antibodies can be prepared by, e.g., the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)), or as taught herein. Human monoclonal antibodies may be utilized in the practice of the presently disclosed and claimed invention and may be produced by using human hybridomas (see Cote, et al., PNAS 80:2026 (1983)) or by transforming human B-cells with Epstein Barr Virus in vitro (see Cole, et al., 1985), relevant portions incorporated herein by reference.

In addition, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example but not by way of limitation, in U.S. Pat. Nos. 5,545,807; 5,545,806; 5,569,825; 5,625,126; 5,633,425; 5,661,016, and in Marks et al., J Biol. Chem. 267:16007, (1992); Lonberg et al., Nature, 368:856 (1994); Morrison, 1994; Fishwild et al., Nature Biotechnol. 14:845 (1996); Neuberger, Nat. Biotechnol. 14:826 (1996); and Lonberg and Huszar, Int Rev Immunol. 13:65 (1995), relevant portions incorporated herein by reference.

A method for producing an antibody of interest, such as a human antibody, is disclosed in U.S. Pat. No. 5,916,771, issued to Hori et al. on Jun. 29, 1999, and incorporated herein by reference. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

As used herein, the term “treatment” refers to both therapeutic treatment and prophylactic or preventative measures. Those in need of treatment include those already with the disorder as well as those in which the disorder is to be prevented.

As used herein, the term “effective amount” refers to the amount of a therapeutic agent (e.g., a peptide of the present invention) sufficient to effect beneficial or desired results. An effective amount can be administered in one or more administrations, applications or dosages and is not intended to be limited to a particular formulation or administration route.

As used herein, the term “disorder” refers to any condition that would benefit from treatment with the polypeptide. This includes chronic and acute disorders or diseases including those infectious or pathological conditions that predispose the mammal to the disorder in question.

An antibody, antigen-binding domain, or antibody fragment can be generated with an engineered sequence or glycosylation state to confer preferred levels of activity in antibody-dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

Alternatively, or additionally, it may be useful to combine amino acid modifications with one or more further amino acid modifications that alter complement component Clq binding and/or the complement-dependent cytotoxicity (CDC) function of the Fc region of an IL-23p19 binding molecule. The binding polypeptide of particular interest may be one that binds to Clq and displays complement-dependent cytotoxicity. Polypeptides with pre-existing Clq binding activity, optionally further having the ability to mediate CDC may be modified such that one or both of these activities are enhanced. Amino acid modifications that alter Clq and/or modify its complement-dependent cytotoxicity function are described, for example, in W0/0042072, which is hereby incorporated by reference.

An Fc region of an antibody can be designed to alter the effector function, e.g., by modifying Clq binding and/or FcγR binding and thereby changing complement-dependent cytotoxicity (CDC) activity and/or antibody-dependent cell-mediated cytotoxicity (ADCC) activity. These “effector functions” are responsible for activating or diminishing a biological activity (e.g., in a subject). Examples of effector functions include, but are not limited to: Clq binding; CDC; Fc receptor binding; ADCC; phagocytosis; down-regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved Clq binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa). Affinity of the Fc domain to FcRn can also be engineered; this has the effect of altering the half-life of the antibody in serum.

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen-binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine, and glycine. However, other residues can function as well. Phage display can be used to rapidly select tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) is displayed on filamentous phage and subjected to affinity selection with hapten. The population of selected variants exhibited significant increases in binding activity but retained considerable sequence diversity. Sequence analysis revealed a conserved proline in the linker two residues after the VH C terminus and an abundance of arginines and prolines at other positions as the only common features of the selected tethers. In certain embodiments, the antibody fragments are further modified to increase their half-life by using modified Fc regions or mutations to the various constant regions, as are known in the art. In certain aspects, the present invention includes novel antibodies with Fc regions that yield extended half-life, e.g., when used in subcutaneous administration.

As used herein, the term “pharmaceutical composition” refers to the combination of an active agent with a carrier, inert or active, making the composition especially suitable for diagnostic or therapeutic use in vitro, in vivo, or ex vivo. As used herein, the term “pharmaceutically acceptable carrier” refers to any of the standard pharmaceutical carriers, such as a buffered solutions, water, emulsions (e.g., such as an oil/water or water/oil emulsions), ionically stabilized solutions, and various types of wetting agents. The compositions also can include stabilizers, preservatives, carriers, stabilizers, and adjuvants. Formulations of anti-myostatin binding molecules can be prepared by mixing the antibodies and/or antigen binding domain(s) or fragment(s) thereof with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Twenty Third Edition, Elsevier, Hardback ISBN: 9780128200070, eBook ISBN: 9780128223895, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y., relevant portions incorporated herein by reference).

In certain embodiments, the antibodies of the present invention are formulated for administration to humans. For example, the antibodies of the present invention can be included in a pharmaceutical composition formulated for an administration that is: intranasal, intrapulmonary, intrabronchial, intravenous, oral, intraadiposal, intraarterial, intraarticular, intracranial, intradermal, intralesional, intramuscular, intrapericardial, intraperitoneal, intrapleural, intravesicular, local, mucosal, parenteral, enteral, subcutaneous, sublingual, topical, transbuccal, transdermal, via inhalation, via injection, in creams, in lipid compositions, via a catheter, via a lavage, via continuous infusion, via infusion, via local delivery, or via localized perfusion, and wherein the composition is a serum, drop, gel, ointment, spray, reservoir, or mist.

As used herein, the term “antigen” refers to a molecule containing one or more epitopes (either linear, conformational or both) that will stimulate a host's immune-system to make a humoral and/or cellular antigen-specific response. The term is used interchangeably with the term “immunogen”. Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “epitope” refers to a specific amino acid sequence or molecule (such as a carbohydrate, small molecule, lipid, etc.) that when present in the proper conformation, provides a reactive site for an antibody (e.g., B cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad Sci. USA 81:3998-4002; Geysen et al. (1986) Molec. Immunol. 23:709-715, relevant portions incorporated herein by reference.

As used herein, the term “substantially purified” refers to isolation of a substance (compound, polynucleotide, protein, polypeptide, polypeptide composition) such that the substance comprises the majority percent of the sample in which it resides. Typically, in a sample a substantially purified component comprises 50%, 80%-85%, 90-95%, or even 95 to 100% of the sample. Techniques for purifying polypeptides and polynucleotides of interest are well-known in the art and include, for example, ion-exchange chromatography, affinity chromatography and sedimentation according to density.

As used herein, the term “treatment” refers to any of (i) the prevention of infection or reinfection, as in a traditional vaccine, (ii) the reduction or elimination of symptoms, and (iii) treating muscle wasting disease(s), increasing the mass of muscle tissue, and/or increasing the strength of muscle tissue. Treatment may be effected prophylactically (prior to muscle dystrophy) or therapeutically (following muscle dystrophy).

The practice of the present invention employs, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference. The nucleic acid sequences encoding the polypeptides disclosed herein can be obtained using any generally available protein to nucleic acid software, and can be optimized for expression in a host cell, e.g., a human sequence and host cell. The nucleic acid sequences are part of the present invention, e.g., www.bioinformatics.org/sms2/rev_trans.html.

The amino acid, nucleic acid, and genomic sequences of full-length human myostatin can be found in the following: HGNC: 4223 NCBI Gene: 2660 Ensembl: ENSG00000138379 OMIM®: 601788 UniProtKB/Swiss-Prot: O14793, and Human Growth/differentiation factor 11 (GDF11) is also used herein, with Uniprot O95390, relevant sequences incorporated herein in their entirety.

Antibody Library Design and Construction. Antibody libraries were designed to optimize the binding characteristics, stability, and humanness of a template antibody. This was achieved by first performing germline annotations for the heavy chain variable domain (VH) and light chain variable domain (VL) of the input antibody. From the germline assignments, the modeled somatic hypermutation probabilities of each possible amino acid substitution at each position in the VH and VL sequences were queried from the cAbRep database (DOI: 10.3389/fimmu.2019.02365).

In order to construct antibody libraries modeled after the somatic hypermutation patterns, the VH sequence was split into three sections and VL sequence into two, resulting in five total library fragments. Each library fragment was diversified with all possible single mutations observed in the somatic hypermutation patterns above a set frequency threshold. Double mutational space was then sampled until 2000 total variants were identified. All library components were enforced to be free of certain amino acid and nucleotide liabilities that might negatively impact the structural integrity of the antibody, or the molecular biology required to assemble the libraries.

Antibody libraries were assembled by a two-step Golden Gate assembly process. First, the individual VH and VL libraries were assembled from their fragments into their own respective plasmids. These plasmids were then combined with a third plasmid encoding for a GGGGSGGGGSGGGGS (SEQ ID NO:267) linker, and an appropriate phagemid backbone, in a second Golden Gate reaction for the assembly of the final single-chain variable fragment library in an appropriate phage display vector.

Phagemids were electroporated into TG-1 bacteria and grown in flasks in 2x YT+2% glucose+100 ug/mL carbenicillin at 37° C. 250 rpm overnight. The following day, cultures were superinfected with M13K07 helper phage, after being grown for another hour at 37 C 250 rpm following a 1:20 dilution in 2x YT growth medium. 100 ug/ml Carbenicillin and 50 ug/ml Kanamycin were added to the cultures 1 hour after superinfection. Cultures were then incubated overnight at 32° C. 250 rpm to facilitate phage production. Phage virion were recovered with a PEG precipitation protocol, eluted into 1×TBS buffer, quantified by spectrophotometry, and diluted as appropriate for storage.

In-Vitro Antibody Selections. Antibody in-vitro selections were carried out with two rounds of phage display, followed by conversion to a mammalian display library for the final round. 5×10¹² virions were used as the input for each phage display round.

In the first round of selection, streptavidin-coated magnetic beads were incubated with biotinylated PSR, followed by incubation with the phage display library. Phages that bound to PSR were captured by the beads and removed using the KingFisher system, while the unbound phages were retained for subsequent positive selection. In the positive selection step, the remaining phage pool was incubated with soluble biotinylated GDF-8 (10 nM) in the presence of unbiotinylated GDF-11 at a concentration of 50 nM. After competitive incubation, streptavidin-coated magnetic beads were added to specifically capture the remaining biotinylated GDF-8 and any bound phages. These GDF-8-bound phages were then separated using the KingFisher system, washed to remove non-specifically bound phages, eluted from the beads, and amplified in bacterial host cells for use in the second round of selection.

In the second round, another round of negative selection was performed against biotinylated PSR using the same method as in the first round. For positive selection, the amplified phage pool was incubated with soluble biotinylated GDF-8 at a concentration of 5 nM, in the presence of a higher concentration of unbiotinylated GDF-11 (200 nM). After the competition step, streptavidin-coated magnetic beads were used to capture the remaining biotinylated GDF-8 and the GDF-8-specific phages. These beads were separated using the KingFisher™ system, washed to remove any non-specifically bound phages, and the GDF-8-bound phages were eluted and amplified for subsequent characterization.

Phagemid from the round 2 selection was collected by midiprep of the transduced E. coli from the phage propagation step. ScFv inserts were transferred into an appropriate mammalian display vector to display the scFv as transmembrane-bound scFv-Fc by PCR with appropriate primers, followed by ligation into the mammalian display vector. ExpiCHO™ cells were then transfected at a 0.25 ug/ml DNA to culture ratio using the Expifectamine™ CHO transfection system to enforce monoclonal expression of the transmembrane scFv-Fc library.

At 48 hours post-transfection, cells were sorted to collect high-expressing, GDF-8-binding clones. Cells were stained with an anti-Fc antibody to quantify expression level, GDF-8, GDF-11, and a viability dye in appropriate colors to enable multi-color cell sorting. The top 0.1% of live, anti-Fc+ cells were sorted into a collection tube. After collection, cells were lysed to collect their RNA, which was reverse transcribed and cloned back into a soluble mammalian expression vector to enable monoclonal screening by SPR kinetics.

Label-Free Binding Kinetics as Measured by SPR. The binding kinetics of antibodies to GDF-8 and GDF-11 were measured using a Carterra LSA. All steps were conducted in 1×HBST+0.05% BSA kinetics buffer. Antibodies were captured on an anti-Human Fc-functionalized HC200M chip for 25 minutes, followed by six buffer injections to establish a baseline. This was followed by a series of injections of either GDF-8 or GDF-11, arranged in order of increasing concentration. Each injection cycle consisted of a baseline, followed by an association phase with GDF-8 or GDF-11, and then a dissociation step in the assay buffer. This was followed by two 40-second injections of 0.1M glycine at pH 2.0 to regenerate the anti-Fc capture surface. Kinetic parameters were then calculated from the data using the LSA Kinetics software.

Antibody Expression and purification. Antibody expression plasmids were transiently introduced into an animal cell line using the ExpiFectamine CHO Transfection Kit (ThermoFisher; Cat #A29129) to yield transfectants that produced antibody. For a host cell line, ExpiCHO-S (ThermoFisher; Cat #A29127) was used. After 6-12 days of growth post introduction of DNA, cell suspensions of ExpiCHO were harvested via centrifugation for 20 min at 4,000×g, and then filtered using 0.2 μm Disposable PES Filter units (FisherScientific, Cat #FB12566504). Antibody was recovered from filtrate using Protein A purification (HiTrap MabSelect SuRe; Cytiva Cat #GE11-0034-93).

HEK293SBE SMAD Signaling Assay. HEK293 cells stably expressing a luciferase reporter gene linked to a SMAD binding element promoter (SBE) (BPS Bioscience, Cat #60653) were cultured in MEM media (Cytiva, Cat #SH30024.01) supplemented with 10% FBS (MilliporeSigma, Cat #F4135), 1% non-essential amino acids (Gibco, Cat #11140-050), 1 mM Na pyruvate (Gibco, Cat #11360-070), 1% Penicillin/Streptomycin (Corning, Cat #30-002-CI), and 400 μg/mL of Geneticin (Gibco, Cat #10131035) according to manufacturer's instructions.

To determine SMAD 2/3 activation, the HEK293/SBE/Luc cells were seeded in white, clear bottom 384-well plate (Greiner Bio-One, Cat #781098) at 3000 cells/well using Assay Media consisting of MEM media supplemented with 0.5% FBS (Gibco, Cat #26400044), 1% non-essential amino acids, 1 mM Na pyruvate, and 1% Penicillin/Streptomycin. 18-24 hours following plating, the cells were treated with the indicated antibodies at final dose range of 0.001-66.67 nM after an one hour, 37° C./5% CO₂, antibody pre-incubation with either Myostatin (PeproTech, Cat #120-00), GDF-11 (PeproTech, Cat #120-11), Activin A (PeproTech, Cat #120-14E), or Activin B (R&D Systems, Cat #11517-AB) at a final concentration of 1 nM for each ligand. Following 18 hours of antibody/ligand incubation, 1 volume of ONE-Step Luciferase reagent (BPS Bioscience, Cat #60690) was added to cells and plates were shaken for 15 minutes at room temperature to facilitate lysis. After 15 minutes, luminescence was read on a SpectraMax ID5 (Molecular Devices) with 1000 ms integration time. GraphPad Prism 10.2.2 was used to calculate IC₅₀ values, generate graphs, and perform statistical analysis.

Myoblast differentiation assay. Primary human myoblast cells were purchased from Cook Myocite (Cat #SK-1111-P01457-24M). The cells were cultured in SkGM-2 Skeletal Muscle Cell Growth Media (Lonza, Cat #CC-3246), supplemented with the Medium-2 BulletKit (Lonza, Cat #CC-3244). Cell passage 3rd-6^{th} were used for this experiment.

To evaluate the effectiveness of anti-myostatin antibodies in rescuing myoblast differentiation attenuated by myostatin, cells were seeded at 10,000 cells per well in a 96-well plate (Corning, high throughput content imaging, cat #4680) and incubated overnight in a cell culture incubator. Cell samples were then treated with the indicated antibodies at a final dose range of 0.0017-100 nM after a one-hour pre-incubation at room temperature with either Myostatin (PeproTech, Cat #120-00), GDF-11 (PeproTech, Cat #120-11), at a final concentration of 10 nM for each ligand. Cell samples were incubated in a cell culture incubator at 37° C. with 5% CO2 for 72 hours. After incubation, the samples were washed twice with 1×PBS and then fixed with 4% paraformaldehyde (Thermo Scientific, Cat #J19943.K2) for 30 minutes at room temperature. The samples were then washed twice with 1×PBS and blocked with blocking buffer (2% FBS, 0.2% Triton X-100 in 1×PBS) for 1 hour at room temperature. Prepare the anti-MF20 antibody (eBioscience, Cat #53-6503-82) by diluting the stock antibody 1:1000 and DAPI 1:10000 in antibody dilution buffer (2% FBS, 0.01% Triton X-100 in 1×PBS). Remove the blocking buffer and add 25 μL/well of the antibody solution. Incubate the samples for 1 hour at room temperature. Remove the antibody solution and wash the samples twice with 1×PBS. The samples are now ready for imaging. Acquire images of the samples using a 20× objective on the ImageExpress (Molecular Devices). Analyze the samples using custom analysis with ImageExpress software. The myoblast differentiation was evaluated by myotube coverage and fusion index. GraphPad Prism 10.2.2 was used to calculate EC₅₀ values and generate graphs.

Differential scanning fluorimetry/dynamic light scattering (DSF/DLS). Melting temperature (DSF) and aggregation (DLS) were measured using the NanoTemper Prometheus Panta nanoDSF instrument. Antibodies were diluted to 1 mg/mL in PBS and added to a 10 μL glass microcapillary tube. The cumulant radius (in nm) and polydispersion index (PDI) of the antibody samples were first determined by making multiple DLS measurements at room temperature. Melting temperature, turbidity, and aggregation as a function of temperature were determined by subjecting samples to a temperature gradient (20-95° C.) over time, plotting the ratio of the absorbances of the sample at 350:330 nm or the cumulant radius against time, and then calculating the inflection points.

Size exclusion chromatography (SEC-HPLC). Size-exclusion chromatography to measure antibody assembly and quality was performed using an Agilent Infinity 1290 HPLC instrument on a TSKgel® UP-SW3000 column (Tosoh Cat #0023449). Antibody samples were first diluted to 1 mg/mL in PBS and added to a 96-well plate. 2 μL of each sample was injected over the column for 10 minutes at 0.25 mL/min using 0.1 M sodium phosphate pH 7 as eluent. Peak integration was performed using the OpenLab Chemstation software and antibody quality was assessed by comparing sample peaks to a gel filtration standard (BioRad, Cat #1511901).

FcRn binding affinity. FcRn binding affinity was measured using biolayer interferometry (BLI) on a ForteBio Octet Red384 instrument. Biotinylated FcRn:beta-2-microglobulin heterodimer (Acro Biosystems, Cat #FCM-H5283) was prepared at 20 μg/mL in PBS+0.01% Tween-20 pH 7.4, then captured on SA Biosensors (Sartorius, Cat #18-5019) for 180 seconds to ≈7 nm. Biosensors were then baselined in acetate buffer at pH 6 for 180 seconds followed by 5-minute sample association and dissociation phases in the same buffer. Regeneration was performed by dipping biosensors in PBS+0.01% Tween-20, pH 7.4 for 30 seconds 3 times. The resulting kinetic profiles were fit using a 1:1 binding model with the Data Analysis HT software.

Capillary gel electrophoresis (CGE). Capillary gel electrophoresis was performed on the Revvity LabChip GXII Touch CGE instrument according to the Protein Express Assay kit instructions (Revvity, Cat #760499). Briefly, samples and ladder were denatured and reduced at 100° C. for 5 minutes in denaturing buffer containing beta-mercaptoethanol on a skirted PCR plate in a thermocycler. Staining and destaining gel as well as the lower marker solution were applied to the LabChip according to the kit instructions for a high-throughput assay. Results were analyzed using the LabChip GX Reviewer software.

Stress testing. Preliminary stability assessments were made of antibodies at 1 mg/ml under several conditions: 1 freeze-thaw cycle (in PBS), 3 freeze-thaw cycles (in PBS), overnight at 55° C. (in PBS), 1 hour at pH 3.2 (in citrate buffer), and 1 hour at pH 4.5 (in acetate buffer). Freeze-thaw cycles were performed by moving antibody samples from −80° C. to room temperature. For accelerated degradation at high temperature, antibody samples were left at 55° C. in a thermocycler for 16 hours. For acid stress, antibodies were exchanged into citrate or acetate buffer for 1 hour and then exchanged back into PBS. The effect of these conditions on antibody quality was measured using SEC-HPLC, DLS/DSF, and CGE as described and compared to antibody samples stored at 4° C.

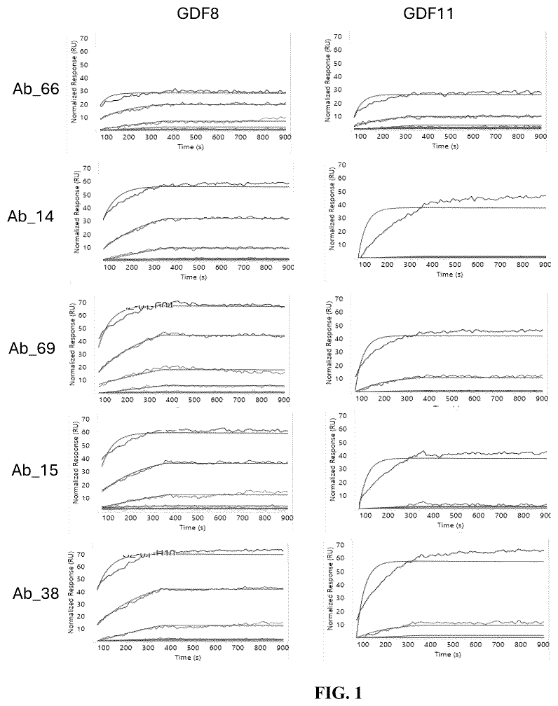

FIG. 1 shows SPR affinity measurements of binding to GDF8 or GDF11 for phage display-selected clones.

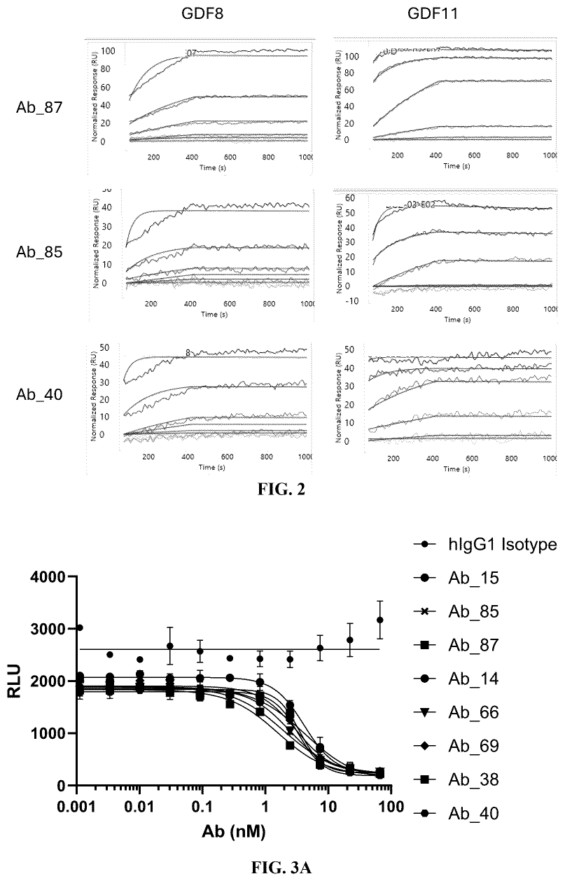

FIG. 2 shows SPR affinity measurements of binding to GDF8 or GDF11 for phage display and mammalian display-selected clones.

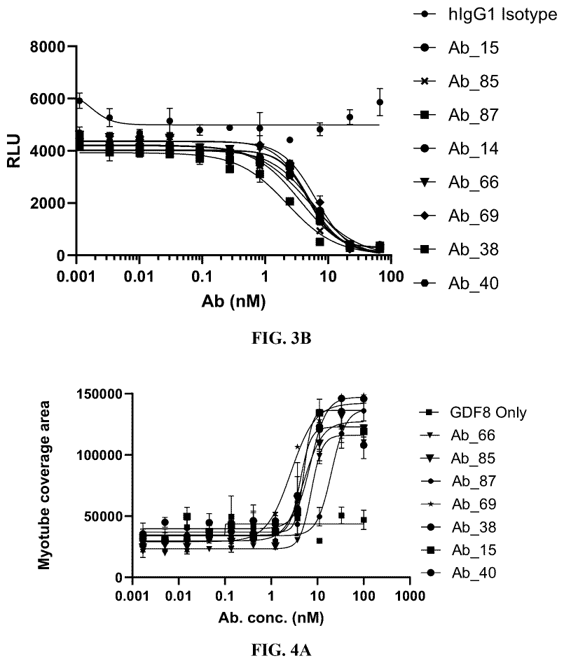

FIG. 3A shows reporter cell line to measure GDF8 antagonism. Antibodies expressed with Fc_1.

FIG. 3B shows reporter cell line to measure GDF11 antagonism. Antibodies expressed with Fc_1.

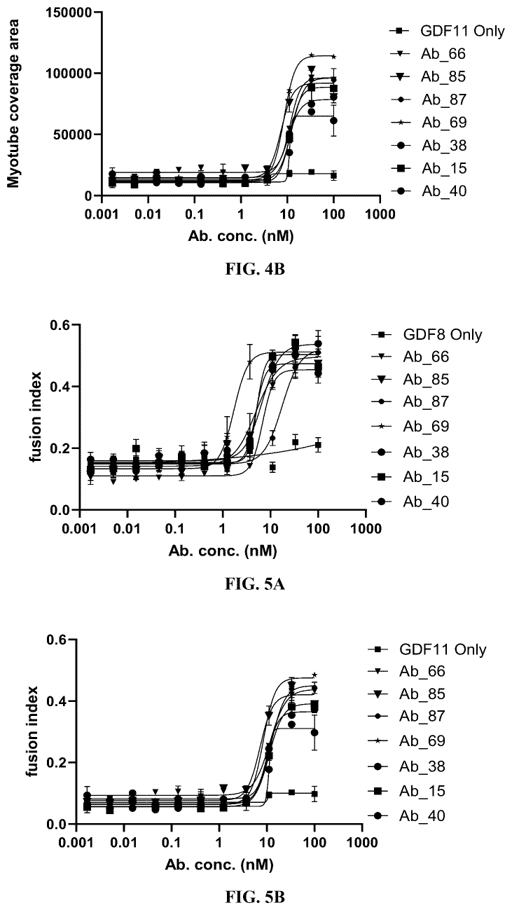

FIG. 4A shows myotube coverage as measure of myoblast differentiation. GDF8 antagonism. Antibodies made with Fc_1.

FIG. 4B shows myotube coverage as measure of myoblast differentiation. GDF11 antagonism. Antibodies made with Fc_1.

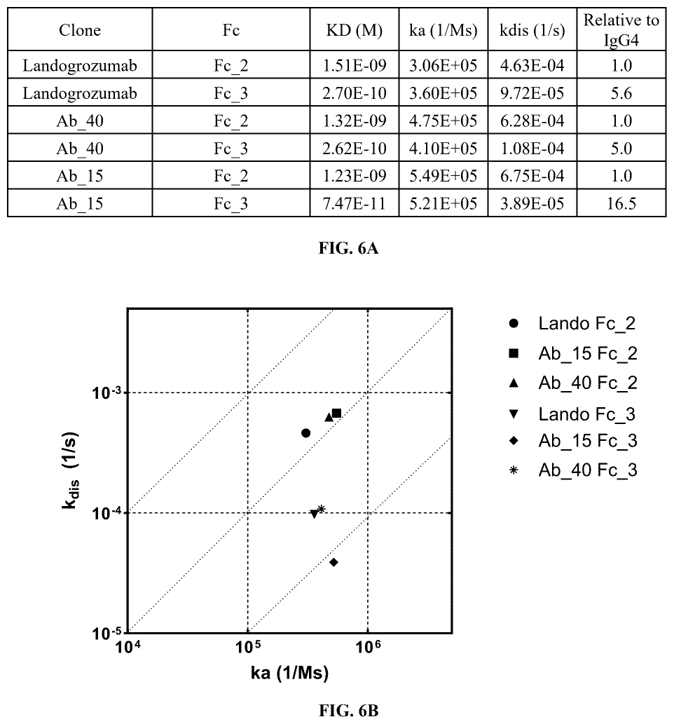

FIG. 5A shows a fusion index as measure of myoblast differentiation. GDF8 antagonism. Antibodies made with Fc_1.

FIG. 5B shows a fusion index as measure of myoblast differentiation. GDF11 antagonism. Antibodies made with Fc_1.

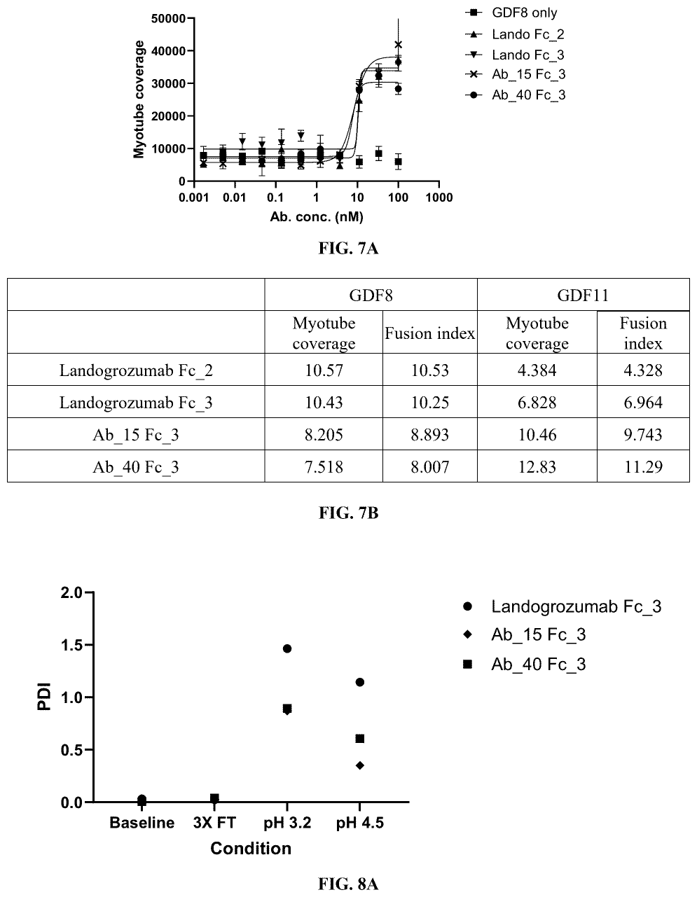

FIG. 6A shows the binding kinetics for antibody and Fc combinations against human FcRn via BLI.

FIG. 6B is an isoaffinity plot of antibody and Fc combinations for binding against human FcRn via BLI.

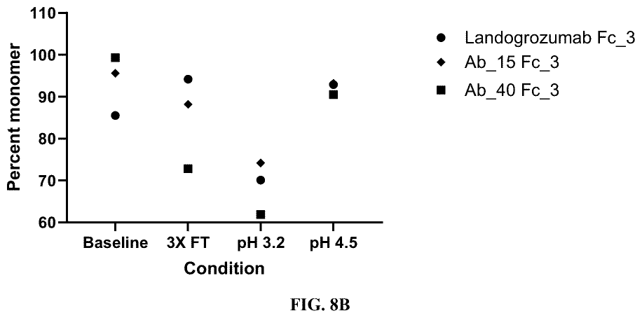

FIG. 7A shows myotube coverage as measure of myoblast differentiation.

FIG. 7B shows the IC₅₀ (nM) for antibody and Fc combinations against GDF8 and GDF11 in myoblast differentiation, for both myotube coverage and fusion index.

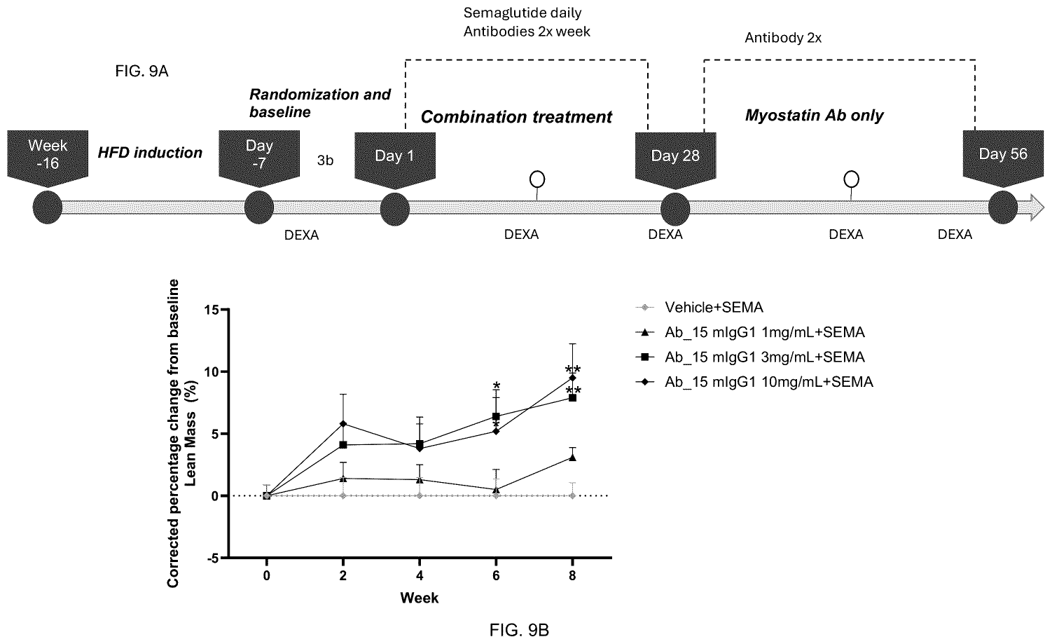

FIG. 8A shows the measure of antibody size distribution by DLS, reported as PDI, after treatment under several stress conditions.

FIG. 8B shows the measure of antibody monomericity by HPLC-SEC, reported as percent monomer.

The skilled artisan will recognize that antibodies which exhibit little or no binding to a target antigen can be described as having a low affinity, and a high equilibrium dissociation constant (KD) for the target antigen. The skilled artisan will also recognize that antibodies which exhibit little or no binding to a collective assembly of target antigenic epitopes can be described as having a low avidity, and a high equilibrium dissociation constant (KD) for the collective assembly of target antigenic epitopes.

In some embodiments, provided herein are anti-myostatin antibodies having a binding affinity (K_{D}) to myostatin of about 5 μM to about 5 pM, about 1 μM to about 5 pM, about 0.5 μM to about 5 pM, about 0.1 μM to about 5 pM, about 50 nM to about 5 pM, about 10 nM to about 5 pM, about 5 nM to about 5 pM, about 1 nM to about 5 pM, about 0.5 nM to about 5 pM, about 0.1 nM to about 5 pM, about 50 pM to about 5 pM, about 10 pM to about 5 pM.

In some embodiments, anti-myostatin antibodies have a binding avidity (K_{D})) to myostatin of about 500 nM to about 0.1 pM, about 100 nM to about 0.1 pM, about 50 nM to about 0.1 pM, about 10 nM to about 0.1 pM, about 5 nM to about 0.1 pM, about 1 nM to about 0.1 pM, about 0.5 nM to about 0.1 pM, about 0.1 nM to about 0.1 pM, about 50 pM to about 0.1 pM, about 10 pM to about 0.1 pM, about 5 pM to about 0.1 pM, about 1 pM to about 0.1 pM, about 0.5 pM to about 0.1 pM.

In some embodiments, anti-myostatin antibodies have a half maximal effective concentration (EC₅₀) to myostatin of about 500 nM to about 0.001 nM, about 100 nM to about 0.001 nM, about 50 nM to about 0.001 nM, about 10 nM to about 0.001 nM, about 5 nM to about 0.001 nM, about 1 nM to about 0.001 nM, about 0.5 nM to about 0.001 nM, about 0.1 nM to about 0.001 nM, about 0.05 nM to about 0.001 nM, about 0.01 nM to about 0.001 nM, about 0.005 nM to about 0.001 nM.

The skilled artisan will recognize that binding specificity may be determined through a series of competition binding paradigms, in which a desired antibody demonstrates its ability to prevent binding of a known refence antibody to its target epitope at varying concentrations. In some embodiments, the reference anti-myostatin antibody is landogrozumab. In some embodiments, the reference antibody, which binds the epitope recognized by landogrozumab. The skilled artisan will also recognize that landogrozumab may be utilized as control antibodies in agonist and antagonist assays.

In some embodiments, the myostatin antagonist antibody is a full-length antibody (referring to an antibody with two heavy and two light chains attached to the Fc domain, giving a ‘Y’ shape). In some embodiments the Fc domain (or simply referred to as an Fc) is a human Fc domain. In some embodiments, the Fc domain of myostatin antagonist antibody is from a human IgG1, human IgG2, human IgG3, or human IgG4, or can be one of the mutated Fc regions taught herein.

### Anti-Myostatin Antibodies—CDR and Amino Acid Sequences

Provided herein are sequences for exemplary myostatin antagonist antibodies of the disclosure. Included are complementarity determining region (CDR) sequences for the variable heavy and light domain sequences (VH, VL) that constitute the myostatin antigen binding domains of the disclosure.

As referred below, a light chain variable (VL) domain CDR1 region is referred to as CDR-L1; a VL CDR2 region is referred to as CDR-L2; a VL CDR3 region is referred to as CDR-L3; a heavy chain variable (VH) domain CDR1 region is referred to as CDR-H1; a VH CDR2 region is referred to as CDR-H2; and a VH CDR3 region is referred to as CDR-H3. Table 1 provides exemplary CDR combinations of antibodies of the disclosure.

TABLE 1
Exemplary Anti-Myostatin Antibody CDR Combinations

| ID | VH | VL | CDRH1 | CDRH2 | CDRH3 | CDRL1 | CDRL2 | CDRL3 |
|---|---|---|---|---|---|---|---|---|
| Ab_03 | EVQLVES | EIVLTQSP | GLTFSRYP | ITSSGG | ARLPDY | SSVSS | STS | QHHS |
| | GGGLVQ | GTLSLSPG | | ST | | SY | | WYHFT |
| | PGGSLRL | ERATLSCR | | | | | | |
| | SCAASGL | ASSSVSSS | | | | | | |
| | TFSRYPM | YLHWYQQ | | | | | | |
| | SWVRQA | KPGQAPR | | | | | | |
| | PGKGLV | LLIYSTSNL | | | | | | |
| | WVSAITS | VAGIPDRF | | | | | | |
| | SGGSTYY | SGSGSGT | | | | | | |
| | SDTVKGR | DFTLTISRL | | | | | | |
| | FTISRDN | EPEDFAVY | | | | | | |
| | AKNTLYL | YCQHHSW | | | | | | |
| | QMNSLR | YHFTFGG | | | | | | |
| | AEDTAVY | GTKVEIK | | | | | | |
| | YCARLPD | | | | | | | |
| | YWGQGT | | | | | | | |
| | LVTVSS | | | | | | | |
| SEQ ID | 1 | 2 | 3 | 4 | 5 | 6 | 7 | 8 |
| NO: | | | | | | | | |
| Ab_42 | EVQLVES | EIVLTQSP | GLTFSRYP | ITSSGG | ARLPDY | SSVSS | STS | QHHSG |
| | GGGLVQ | GTLSLSPG | | ST | | SY | | YHFT |
| | PGGSLRL | ERATLSCR | | | | | | |
| | SCAASGL | ASSSVSSS | | | | | | |
| | TFSRYPM | YLHWYQQ | | | | | | |
| | SWVRQA | KPGQAPR | | | | | | |
| | PGKGLV | LLIYSTSNL | | | | | | |
| | WVSAITS | VAGIPDRF | | | | | | |
| | SGGSTYY | SGSGSGT | | | | | | |
| | SDTVKGR | DFTLTISRL | | | | | | |
| | FTISRDN | EPEDFAVY | | | | | | |
| | AKATLYL | YCQHHSG | | | | | | |
| | QMNSLR | YHFTFGG | | | | | | |
| | AEDTAVY | GTKVEIK | | | | | | |
| | YCARLPD | | | | | | | |
| | YWGQGT | | | | | | | |
| | LVTVSS | | | | | | | |
| SEQ ID | 11 | 12 | 13 | 14 | 15 | 16 | 17 | 18 |
| NO: | | | | | | | | |
| Ab_65 | EVQLLES | DIQMTQS | GFTFSSYA | ISSGGS | AKQVYA | QDVS | TAS | QQHYS |
| | GGGLVQ | PSSLSASV | | YT | MNY | TA | | TPWT |
| | PGGSLRL | GDRVTITC | | | | | | |
| | SCAASGF | KASQDVS | | | | | | |
| | TFSSYAM | TAVAWYQ | | | | | | |
| | SWVRQA | QKPGKAP | | | | | | |
| | PGKGLE | KLLIYTASY | | | | | | |
| | WVSTISS | RETGVPSR | | | | | | |
| | GGSYTSY | FSGSGSGT | | | | | | |
| | PDSVKGR | DFTLTISSL | | | | | | |
| | FTISRDNS | QPEDFATY | | | | | | |
| | KNTLYLQ | YCQQHYS | | | | | | |
| | MNSLRA | TPWTFGG | | | | | | |
| | EDTAVYY | GTKVEIK | | | | | | |
| | CAKQVY | | | | | | | |
| | AMNYW | | | | | | | |
| | GQGTLVT | | | | | | | |
| | VSS | | | | | | | |
| SEQ ID | 21 | 22 | 23 | 24 | 25 | 26 | 27 | 28 |
| NO: | | | | | | | | |
| Ab_86 | EVQLLES | DIQMTQS | GFTFSSYA | ISSGGS | AKQDYA | QDVS | SAS | QQHYI |
| | GGGLVQ | PSSLSASV | | YT | MNY | TA | | TPWT |
| | TGGSLRL | GDRVTITC | | | | | | |
| | SCAASGF | KSSQDVST | | | | | | |
| | TFSSYAM | AVAWYQ | | | | | | |
| | SWVRQA | QKPGKAP | | | | | | |
| | PGKGLE | KLLIYSASY | | | | | | |
| | WVSTISS | RYTGVPSR | | | | | | |
| | GGSYTSY | FSGSGSGT | | | | | | |
| | PDSVKGR | DFTLSISSL | | | | | | |
| | FTISRDNS | QPEDFATY | | | | | | |
| | KNTLYLQ | YCQQHYIT | | | | | | |
| | MNSLRA | PWTFGGG | | | | | | |
| | EDTAVYY | TKVEIK | | | | | | |
| | CAKQDY | | | | | | | |
| | AMNYW | | | | | | | |
| | GQGTLVT | | | | | | | |
| | VSS | | | | | | | |
| SEQ ID | 31 | 32 | 33 | 34 | 35 | 36 | 37 | 38 |
| NO: | | | | | | | | |
| Ab_06 | EVQLVES | EIVLTQSP | GLTFSRYP | ITSSGG | ARLPDY | SSVSK | STS | QHHSG |
| | GGGLVQ | GTLSLSPG | | ST | | SY | | PHGT |
| | PGGSLRL | ERATLSCR | | | | | | |
| | SCAASGL | ASSSVSKS | | | | | | |
| | TFSRYPM | YLHWYQQ | | | | | | |
| | SWVRQA | KPGQAPR | | | | | | |
| | PGKGLV | LLIYSTSNL | | | | | | |
| | WVSAITS | VAGIPDRF | | | | | | |
| | SGGSTYY | SGSGSGT | | | | | | |
| | SDTVKGR | DFTLTISRL | | | | | | |
| | FTISRDN | EPEDFAVY | | | | | | |
| | AKNTLYL | YCQHHSG | | | | | | |
| | QMNSLR | PHGTFGG | | | | | | |
| | AEDTAVY | GTKVEIK | | | | | | |
| | YCARLPD | | | | | | | |
| | YWGQGT | | | | | | | |
| | LVTVSS | | | | | | | |
| SEQ ID | 41 | 42 | 43 | 44 | 45 | 46 | 47 | 48 |
| NO: | | | | | | | | |
| Ab 43 | EVQLVES | EIVLTQSP | GLTFSNYP | ITSSGG | ARLPDY | SSVSS | STS | QHESG |
| | GGGLVQ | GTLSLSPG | | ST | | SY | | YHFT |
| | PGGSLRL | ERATLSCR | | | | | | |
| | SCTASGL | ASSSVSSS | | | | | | |
| | TFSNYPM | YLHWYQQ | | | | | | |
| | SWVRQA | KPGQAPR | | | | | | |
| | PGKGLV | LLIYSTSNL | | | | | | |
| | WVSAITS | VAGIPDRF | | | | | | |
| | SGGSTYY | SGSGSGT | | | | | | |
| | SDTVKGR | DFTLTISRL | | | | | | |
| | FTISRDN | EPEDFAVY | | | | | | |
| | AKNTLYL | YCQHESG | | | | | | |
| | QMNSLR | YHFTFGG | | | | | | |
| | AEDTAVY | GTKVEIK | | | | | | |
| | YCARLPD | | | | | | | |
| | YWGQGT | | | | | | | |
| | LVTVSS | | | | | | | |
| SEQ ID | 51 | 52 | 53 | 54 | 55 | 56 | 57 | 58 |
| NO: | | | | | | | | |
| Ab_66 | EVQLLES | DIQMTQS | GFSFSSYA | ISSGGS | AKQDYA | QDVS | SAS | QQHYS |
| | GGGLVQ | PSSLSASV | | YT | MNY | TA | | TPWT |
| | PGGSLRL | GDRVTITC | | | | | | |
| | SCAASGF | KASQDVS | | | | | | |
| | SFSSYAM | TAVAWYQ | | | | | | |
| | SWVRQA | QKPGKAP | | | | | | |
| | PGKGLE | KLLIYSASY | | | | | | |
| | WVSTISS | RLTGVPSR | | | | | | |
| | GGSYTSY | FSGSGSGT | | | | | | |
| | PDSVKGR | DFTLTISSL | | | | | | |
| | FTISRDNS | QPEDFATY | | | | | | |
| | KNTLYLQ | YCQQHYS | | | | | | |
| | MNSLRA | TPWTFGG | | | | | | |
| | EDTAVYY | GTKVEIK | | | | | | |
| | CAKQDY | | | | | | | |
| | AMNYW | | | | | | | |
| | GQGTLVT | | | | | | | |
| | VSS | | | | | | | |
| | 61 | 62 | 63 | 64 | 65 | 66 | 67 | 68 |
| Ab_87 | EVQLLES | DIQMTQS | GFTFSSYA | ISSGGS | AKQDYA | QDVS | SAS | QQHYS |
| | GGGLVQ | PSSLSASV | | YT | MNY | TA | | TPWT |
| | PGGSLRL | GDRVTITC | | | | | | |
| | SCVASGF | KASQDVS | | | | | | |
| | TFSSYAM | TAVAWYQ | | | | | | |
| | SWVRQA | QKPGKAP | | | | | | |
| | PGKGLE | KLLIYSASY | | | | | | |
| | WVSTISS | RETGVPSR | | | | | | |
| | GGSYTSY | FSGSGSGT | | | | | | |
| | PDSVKGR | DFTLTISSL | | | | | | |
| | FTISRDNS | QPEDFATY | | | | | | |
| | KNTLYLQ | YCQQHYS | | | | | | |
| | MNSLRA | TPWTFGG | | | | | | |
| | EDTAVYY | GTKVEIK | | | | | | |
| | CAKQDY | | | | | | | |
| | AMNYW | | | | | | | |
| | GQGTLVT | | | | | | | |
| | VSS | | | | | | | |
| SEQ ID | 71 | 72 | 73 | 74 | 75 | 76 | 77 | 78 |
| NO: | | | | | | | | |
| Ab_14 | EVQLVES | EIVLTQSP | GLTFSRYP | ITSSGG | ARLPDY | SSVSS | STS | QHHSG |
| | GGGLVQ | GTLSLSPG | | GT | | SY | | YPFT |
| | PGGSLRL | ERATLSCR | | | | | | |
| | SCAASGL | ASSSVSSS | | | | | | |
| | TFSRYPM | YLHWYQQ | | | | | | |
| | SWVRQA | KPGQAPR | | | | | | |
| | PGKGLV | LLIYSTSNL | | | | | | |
| | WVSTITS | VAGIPDRF | | | | | | |
| | SGGGTYY | SGSGSGT | | | | | | |
| | SDTVKGR | DYTLTISRL | | | | | | |
| | FTISRDN | EPEDFAVY | | | | | | |
| | AKNTLYL | YCQHHSG | | | | | | |
| | QMNSLR | YPFTFGGG | | | | | | |
| | AEDTAVY | TKVEIK | | | | | | |
| | YCARLPD | | | | | | | |
| | YWGQGT | | | | | | | |
| | LVTVSS | | | | | | | |
| SEQ ID | 81 | 82 | 83 | 84 | 85 | 86 | 87 | 88 |
| NO: | | | | | | | | |
| Ab_44 | EVQLVES | EIVLTQSP | GLTFGRYP | ITSSGG | ARLPDY | SSLSS | STS | QHHSG |
| | GGGLVQ | GTLSLSPG | | ST | | SH | | YHFT |
| | PGGSLRL | ERVTLSCR | | | | | | |
| | SCAASGL | ASSSLSSS | | | | | | |
| | TFGRYP | HLHWYQ | | | | | | |
| | MSWVR | QKPGQAP | | | | | | |
| | QAPGKG | RLVIYSTSN | | | | | | |
| | LVWVSAI | LVAGIPDR | | | | | | |
| | TSSGGST | FSGSGSGT | | | | | | |
| | YYSDTVK | DFTLTISRL | | | | | | |
| | GRFTISR | EPEDFAVY | | | | | | |
| | DNAKTTL | YCQHHSG | | | | | | |
| | YLQMNS | | | | | | | |
| | LRAEDTA | YHFTFGG | | | | | | |
| | IYYCARLP | GTKVEIK | | | | | | |
| | DYWGQG | | | | | | | |
| | TLVTVSS | | | | | | | |
| SEQ ID | 91 | 92 | 93 | 94 | 95 | 96 | 97 | 98 |
| NO: | | | | | | | | |
| Ab_69 | EVQLLES | DIQMTQS | GFTFSSYA | ISSGGS | AKQDYA | QDVS | SAS | QQHYS |
| | GGGLVQ | PSSLSASV | | YT | MDY | TA | | TPWT |
| | PGGSLRL | GDRVTITC | | | | | | |
| | SCAASGF | KASQDVS | | | | | | |
| | TFSSYAM | TAVAWYQ | | | | | | |
| | SWVRQA | QKPGKAP | | | | | | |
| | PGKGLE | KLLMYSAS | | | | | | |
| | WVSTISS | YRYTGVPS | | | | | | |
| | GGSYTSY | RFSGSGSG | | | | | | |
| | PDSVKGR | TDFTLTIIN | | | | | | |
| | FTISRDNS | LQPEDFAT | | | | | | |
| | KNTLYLQ | YYCQQHY | | | | | | |
| | MNSLRA | STPWTFG | | | | | | |
| | EDTAVYY | GGTKVEIK | | | | | | |
| | CAKQDY | | | | | | | |
| | AMDYW | | | | | | | |
| | GQGTLVT | | | | | | | |
| | VSS | | | | | | | |
| SEQ ID | 101 | 102 | 103 | 104 | 105 | 106 | 107 | 108 |
| NO: | | | | | | | | |
| Ab_15 | EVQLVES | EIVLTQSP | GLTFSRYP | ITSSGG | ARLPDY | SSVSS | ATS | QHFSG |
| | GGGLVQ | GTLSLSPG | | ST | | SY | | YHFT |
| | PGGSLRL | ERATLSCR | | | | | | |
| | SCAASGL | ASSSVSSS | | | | | | |
| | TFSRYPM | YLHWYQQ | | | | | | |
| | SWVRQA | KPGQAPR | | | | | | |
| | PGKGLV | LLIYATSNL | | | | | | |
| | WVSAITS | VAGIPDRF | | | | | | |
| | SGGSTYY | SGSGSGT | | | | | | |
| | SDTVKGR | DFTLTISRL | | | | | | |
| | FTISRDN | EPEDFAVY | | | | | | |
| | AKNTLYL | YCQHFSG | | | | | | |
| | QMNSLR | YHFTFGG | | | | | | |
| | AEDTAVY | GTKVEIK | | | | | | |
| | YCARLPD | | | | | | | |
| | YWGQGT | | | | | | | |
| | LVTVSS | | | | | | | |
| SEQ ID | 111 | 112 | 113 | 114 | 115 | 116 | 117 | 118 |
| NO: | | | | | | | | |
| Ab 45 | EVQLVES | EIVLTQSP | GLTFSRYP | ITSSGG | ARLPDY | SSVSS | ATS | QHHSG |
| | GGGLVQ | GTLSLSPG | | ST | | SY | | YHFT |
| | PGGSLRL | ERATLSCR | | | | | | |
| | SCAASGL | ASSSVSSS | | | | | | |
| | TFSRYPM | YLHWYQQ | | | | | | |
| | SWVRQA | KPGQAPR | | | | | | |
| | PGKGLV | LLIYATSNL | | | | | | |
| | WVSAITS | VAGIPDRF | | | | | | |
| | SGGSTYY | SGSGSGT | | | | | | |
| | SDTVKGR | DFTLTISRL | | | | | | |
| | FTISRDN | EPEDFAVY | | | | | | |
| | AKNTLYL | YCQHHSG | | | | | | |
| | QMNSLT | YHFTFGG | | | | | | |
| | AEDTAVY | GTKVEIK | | | | | | |
| | YCARLPD | | | | | | | |
| | YWGQGT | | | | | | | |
| | LVTVSS | | | | | | | |
| SEQ ID | 121 | 122 | 123 | 124 | 125 | 126 | 127 | 128 |
| NO: | | | | | | | | |
| Ab 81 | EVQLLES | DIQMTQS | GFTFSSYA | ISSGGS | AKQDYA | QDVT | SAS | QQHYS |
| | GGGLVQ | PSSLSASV | | YT | MNQ | TA | | TPWT |
| | PGGSLRL | GDRVSITC | | | | | | |
| | SCAASGF | KASQDVT | | | | | | |
| | TFSSYAM | TAVAWYQ | | | | | | |
| | SWVRQA | QKRGKAP | | | | | | |
| | PGKGLE | KLLIYSASY | | | | | | |
| | WVSTISS | RYTGVPSR | | | | | | |
| | GGSYTSY | FSGSGSGT | | | | | | |
| | PDSVKGR | DFTLTISSL | | | | | | |
| | FTISRDNS | QREDFATY | | | | | | |
| | KNTLYLQ | YCQQHYS | | | | | | |
| | MNSLRA | TPWTFGG | | | | | | |
| | EDTAVYY | GTKVEIK | | | | | | |
| | CAKQDY | | | | | | | |
| | AMNQW | | | | | | | |
| | GQGTLVT | | | | | | | |
| | VSS | | | | | | | |
| SEQ ID | 131 | 132 | 133 | 134 | 135 | 136 | 137 | 138 |
| NO: | | | | | | | | |
| Ab_38 | EVQLVES | EIVLTQSP | GLTFSRYP | ITSSGG | ARLPDY | SSVSS | STS | QHHSG |
| | GGGLVQ | GTLSLSPG | | ST | | SQ | | SHFT |
| | PGGSLRL | ERATLSCR | | | | | | |
| | SCAASGL | ASSSVSSS | | | | | | |
| | TFSRYPM | QLHWYQ | | | | | | |
| | SWVRQA | QKPGQAP | | | | | | |
| | PGKGLV | RLLIYSTSN | | | | | | |
| | WVSAITS | LVAGIPDR | | | | | | |
| | SGGSTYY | FSGSGSGT | | | | | | |
| | SDTVKGR | DFTLTISRL | | | | | | |
| | FTISRDN | EPEDFAVY | | | | | | |
| | AKNTLYL | YCQHHSG | | | | | | |
| | QMNSLR | SHFTFGG | | | | | | |
| | AEDTAVY | GTKVEIK | | | | | | |
| | YCARLPD | | | | | | | |
| | YWGQGT | | | | | | | |
| | LVTVSS | | | | | | | |
| SEQ ID | 141 | 142 | 143 | 144 | 145 | 146 | 147 | 148 |
| NO: | | | | | | | | |
| Ab_46 | EVQLVES | EIVLTQSP | GLTFSRYP | ITSSGG | ARLPDY | SSVSS | STS | QHSSG |
| | GGDLVQ | GTLSLSPG | | ST | | SY | | YHIT |
| | PGGSLRL | ERATLSCR | | | | | | |
| | SCATSGL | ASSSVSSS | | | | | | |
| | TFSRYPM | YLHWYQQ | | | | | | |
| | SWVRQA | KPGQAPR | | | | | | |
| | PGKGLV | LLIYSTSNL | | | | | | |
| | WVSAITS | VAGIPDRF | | | | | | |
| | SGGSTYY | SGSGSGT | | | | | | |
| | SDTVKGR | DFTLTISRL | | | | | | |
| | FTISRDN | EPEDFAVY | | | | | | |
| | AKNTLYL | YCQHSSG | | | | | | |
| | QMNSLR | YHITFGGG | | | | | | |
| | AEDTAVY | TKVEIK | | | | | | |
| | YCARLPD | | | | | | | |
| | YWGQGT | | | | | | | |
| | LVTVSS | | | | | | | |
| SEQ ID | 151 | 152 | 153 | 154 | 155 | 156 | 157 | 158 |
| NO: | | | | | | | | |
| Ab_82 | EVQLLES | DIQMTQS | GFTFSSYA | ISSGGS | AKQDYA | QDVS | SAS | QQHYS |
| | GGGLVQ | PSSLSASV | | YT | MNY | TA | | TPWT |
| | PGGSLRL | GDRVTITC | | | | | | |
| | SCAASGF | KASQDVS | | | | | | |
| | TFSSYAM | TAVAWYQ | | | | | | |
| | SWVRQA | QKPGKAP | | | | | | |
| | PGKGLE | KLLIYSASY | | | | | | |
| | WVSTISS | RYTGVPSR | | | | | | |
| | GGSYTSY | FSGSGSGT | | | | | | |
| | PDSVKGR | DFTLTISSL | | | | | | |
| | FTISRDNS | LPEDFATY | | | | | | |
| | KNTLYLQ | YCQQHYS | | | | | | |
| | MNSLRA | TPWTFGG | | | | | | |
| | EDTAVYY | GTKVEIK | | | | | | |
| | CAKQDY | | | | | | | |
| | AMNYW | | | | | | | |
| | GQGTLVT | | | | | | | |
| | VSS | | | | | | | |
| SEQ ID | 161 | 162 | 163 | 164 | 165 | 166 | 167 | 168 |
| NO: | | | | | | | | |
| Ab_39 | EVQLVES | EIVLTQSP | GFTVSSYG | IRGSD | ARLPDY | SAVSG | STS | QHHSD |
| | GGGLVQ | GTQSLSPG | | GST | | SY | | YHYT |
| | PGGSLRL | ERATLSCR | | | | | | |
| | SCAASGF | ASSAVSGS | | | | | | |
| | TVSSYG | YLHWYQQ | | | | | | |
| | MSWVR | KPGQAPR | | | | | | |
| | QAPGKG | LLIYSTSNL | | | | | | |
| | LEWVSGI | VAGIPDRF | | | | | | |
| | RGSDGST | SGSGSGT | | | | | | |
| | YYADSVK | DFTLTITRL | | | | | | |
| | GRFTISR | EPEDFAVY | | | | | | |
| | DNSKNTL | YCQHHSD | | | | | | |
| | YLQMNS | YHYTFGG | | | | | | |
| | LRAEDTA | GTKVEIK | | | | | | |
| | VYYCARL | | | | | | | |
| | PDYWGQ | | | | | | | |
| | GTLVTVS | | | | | | | |
| | S | | | | | | | |
| SEQ ID | 171 | 172 | 173 | 174 | 175 | 176 | 177 | 178 |
| NO: | | | | | | | | |
| Ab_47 | EVQLVES | EIVLTQSP | GLTFSRYP | ITSSGG | ARLPDY | SSVSS | STS | QHHST |
| | GGGLVQ | GILSLSPGE | | ST | | SY | | PHFT |
| | PGGSLRL | RVTLSCRA | | | | | | |
| | SCAASGL | SSSVSSSYL | | | | | | |
| | TFSRYPM | HWYQQK | | | | | | |
| | SWVRQA | PGQAPRLL | | | | | | |
| | PGKGLV | IYSTSNLV | | | | | | |
| | WVSSITS | AGIPDRFS | | | | | | |
| | SGGSTDY | GSGSGTEF | | | | | | |
| | SDTVKGR | TLTISRLEP | | | | | | |
| | FTISRDN | EDFAVYYC | | | | | | |
| | AKNTLYL | QHHSTPH | | | | | | |
| | QMNSLR | FTFGGGTK | | | | | | |
| | AEDTAVY | VEIK | | | | | | |
| | YCARLPD | | | | | | | |
| | YWGQGT | | | | | | | |
| | LVTVSS | | | | | | | |
| SEQ ID | 181 | 182 | 183 | 184 | 185 | 186 | 187 | 188 |
| NO: | | | | | | | | |
| Ab_83 | EVQLLES | DIQMTQS | GFTFSSNA | ISSGGS | AKQDYA | QDVS | SAS | QQHYS |
| | GGGLVQ | PSSLSASV | | YT | MNY | DA | | TPWT |
| | PGGSLRL | GDRVTITC | | | | | | |
| | SCAASGF | KASQDVS | | | | | | |
| | TFSSNA | DAVAWY | | | | | | |
| | MSWVR | QQTPGKA | | | | | | |
| | QAPGKG | PKLLIYSAS | | | | | | |
| | LEWVSTI | YRPTGVPS | | | | | | |
| | SSGGSYT | RFSGSGSG | | | | | | |
| | SYPDSVK | TDFTLTISS | | | | | | |
| | GRFTISR | LQPEDFAT | | | | | | |
| | DNSKNTL | YYCQQHY | | | | | | |
| | YLQMNS | STPWTFG | | | | | | |
| | LRAEDTA | GGTKVEIK | | | | | | |
| | VYYCAKQ | | | | | | | |
| | DYAMNY | | | | | | | |
| | WGQGTL | | | | | | | |
| | VTVSS | | | | | | | |
| SEQ ID | 191 | 192 | 193 | 194 | 195 | 196 | 197 | 198 |
| NO: | | | | | | | | |
| Ab_40 | EVQLVES | EIVLTQSP | GLTFSRYP | ITSSGG | VRLPDY | SIVSSS | STF | QHHSG |
| | GGGLVQ | GTLSLSPG | | ST | | Y | | YPFT |
| | PGGSLRL | ERATLSCR | | | | | | |
| | SCAASGL | ASSIVSSSY | | | | | | |
| | TFSRYPM | LHWYQQK | | | | | | |
| | SWVRQA | PGQAPRLL | | | | | | |
| | PGKGLV | IYSTFNLV | | | | | | |
| | WVSAITS | ADIPDRFS | | | | | | |
| | SGGSTYY | GSGSGTD | | | | | | |
| | SDTVKGR | FTLTISRLE | | | | | | |
| | FTISRDN | PEDFAVYY | | | | | | |
| | AKNTLYL | CQHHSGY | | | | | | |
| | QMNSLR | PFTFGGGT | | | | | | |
| | AEDTAIY | KVEIK | | | | | | |
| | YCVRLPD | | | | | | | |
| | YWGQGT | | | | | | | |
| | LVTVSS | | | | | | | |
| SEQ ID | 201 | 202 | 203 | 204 | 205 | 206 | 207 | 208 |
| NO: | | | | | | | | |
| Ab_56 | EVQLLES | DIQMTQS | GFTFSSYA | ISSGGS | AKQDYA | QDVS | SAS | QQHYS |
| | GGGLVQ | PSSLSASV | | YT | MNY | TA | | TPWT |
| | PGGSLRL | GDRVTITC | | | | | | |
| | SCAASGF | KASQDVS | | | | | | |
| | TFSSYAM | TAVAWYQ | | | | | | |
| | SWVRQA | QKPGKAP | | | | | | |
| | PGKGLE | KLLIYSASY | | | | | | |
| | WVSTISS | RYTGVPSR | | | | | | |
| | GGSYTSY | FSGSGSGT | | | | | | |
| | PDSVKGR | DFTLTISSL | | | | | | |
| | FTISRDNS | QPEDFATY | | | | | | |
| | KNTLYLQ | HCQQHYS | | | | | | |
| | MNSLRA | TPWTFGG | | | | | | |
| | EDTAVYY | GTKVEIK | | | | | | |
| | CAKQDY | | | | | | | |
| | AMNYW | | | | | | | |
| | GQGTLVT | | | | | | | |
| | VSS | | | | | | | |
| SEQ ID | 211 | 212 | 213 | 214 | 215 | 216 | 217 | 218 |
| NO: | | | | | | | | |
| Ab_84 | EVQLLES | DIQMTQS | GFTFSSYA | ISSGGS | ARQDYA | QDVS | SAS | QQHYS |
| | GGGLVQ | PSSLSASV | | YT | MNY | TA | | TPWT |
| | PGGSLRL | GDRVTITC | | | | | | |
| | SCAASGF | KASQDVS | | | | | | |
| | TFSSYAM | TAVAWYQ | | | | | | |
| | SWVRQA | QKPGKAP | | | | | | |
| | PGKGLE | NLLIYSASY | | | | | | |
| | WVSTISS | RYTGVPSR | | | | | | |
| | GGSYTSY | FSGSGSGT | | | | | | |
| | PDSVKGR | DFTLTISSL | | | | | | |
| | FTISRDNS | QPEDFATY | | | | | | |
| | KNTLYLQ | YCQQHYS | | | | | | |
| | MNSLRG | TPWTFGG | | | | | | |
| | EDTALYY | GTKVEIK | | | | | | |
| | CARQDY | | | | | | | |
| | AMNYW | | | | | | | |
| | GQGTLVT | | | | | | | |
| | VSS | | | | | | | |
| SEQ ID | 221 | 222 | 223 | 224 | 225 | 226 | 227 | 228 |
| NO: | | | | | | | | |
| Ab_41 | EVQLVES | EIVLTQSP | GLTFSRYP | ITSSGG | ARLPDY | SSVSIS | STS | QHESG |
| | GGGLVQ | GTLSLSPG | | ST | | Y | | YHFT |
| | PGGSLRL | ERATLSCR | | | | | | |
| | SCAASGL | ASSSVSISY | | | | | | |
| | TFSRYPM | LHWYQVK | | | | | | |
| | SWVRQA | PGQAPRLL | | | | | | |
| | PGKGLV | IYSTSNLV | | | | | | |
| | WVSAITS | AGIPDRFS | | | | | | |
| | SGGSTYY | GSGSGTD | | | | | | |
| | SDTVKGR | FTLTISRLE | | | | | | |
| | FTISRDN | PEDFAVYF | | | | | | |
| | AKNTLFL | CQHESGY | | | | | | |
| | QMNSLR | HFTFGGG | | | | | | |
| | AEDTAVY | TKVEIK | | | | | | |
| | YCARLPD | | | | | | | |
| | YWGQGT | | | | | | | |
| | LVTVSS | | | | | | | |
| SEQ ID | 231 | 232 | 233 | 234 | 235 | 236 | 237 | 238 |
| NO: | | | | | | | | |
| Ab 63 | EVQLLES | DIQMTQS | GFTFHSYA | ISSGGS | AKQDYA | SQDV | YSA | CQQHY |
| | GGGLVQ | PSSLSASV | | YT | MDL | ST | | STPWT |
| | PGRSLRL | GDRVTITC | | | | | | |
| | SCAASGF | KASQDVS | | | | | | |
| | TFHSYA | TAVAWYQ | | | | | | |
| | MSWVR | QKPGKAP | | | | | | |
| | QAPGKG | KLLMYSAS | | | | | | |
| | LEWVSTI | YRRTGVPS | | | | | | |
| | SSGGSYT | RFSGSGSG | | | | | | |
| | SYPDSVK | TDFTLTISS | | | | | | |
| | GRFTISR | LQPEDVA | | | | | | |
| | DNSKNTL | TYYCQQH | | | | | | |
| | YLQMNS | YSTPWTF | | | | | | |
| | LRAEDTA | GGGTKVEI | | | | | | |
| | VYYCAKQ | K | | | | | | |
| | DYAMDL | | | | | | | |
| | WGQGTL | | | | | | | |
| | VTVSS | | | | | | | |
| SEQ ID | 241 | 242 | 243 | 244 | 245 | 246 | 247 | 248 |
| NO: | | | | | | | | |
| Ab_85 | EVQLLES | DIQMTQS | GFTFSSYA | ISSGGS | AKQDYA | QDVS | SAS | QQHYS |
| | GGGLVQ | PSSLSASV | | YT | MNY | TA | | TPWT |
| | PGGSLRL | GDRVTITC | | | | | | |
| | SCAASGF | KASQDVS | | | | | | |
| | TFSSYAM | TAVAWYQ | | | | | | |
| | SWVRQA | QKPGKAP | | | | | | |
| | PGKGLE | KLLIYSASY | | | | | | |
| | WVSTISS | RYTGVPSR | | | | | | |
| | GGSYTSY | FSGSGSGT | | | | | | |
| | PDSVKGR | DFTLTISRL | | | | | | |
| | FTISRDNS | QPEDFATY | | | | | | |
| | KNTLYLQ | YCQQHYS | | | | | | |
| | MNSLRA | TPWTFGG | | | | | | |
| | EDTAVYY | GTKVEIK | | | | | | |
| | CAKQD- | | | | | | | |
| | AMNYW | | | | | | | |
| | GQGTLVT | | | | | | | |
| | VSS | | | | | | | |
| SEQ ID | 251 | 252 | 253 | 254 | 255 | 256 | 257 | 258 |
| NO: | | | | | | | | |

### Nucleic Acid Sequences

TABLE 3
Nucleic Acid Sequences

| ID | Heavy Chain nucleic acid sequence | Light chain nucleic acid sequence |
|---|---|---|
| Ab_03 | gaagtgcagctggtggaaagcggcggcggcctggtgcagc | gaaattgtgctgacccagagcccgggcaccctgagcc |
| | cgggcggcagcctgcgcctgagctgcgcggcgagcggcct | tgagcccgggcgaacgcgcgaccctgagctgccgcg |
| | gacctttagccgctatccgatgagctgggtgcgccaggcgc | cgagcagcagcgtgagcagcagctatctgcattggta |
| | cgggcaaaggcctggtgtgggtgagcgcgattaccagcag | tcagcagaaaccgggccaggcgccgcgcctgctgatt |
| | cggcggcagcacctattatagcgataccgtgaaaggccgc | tatagcaccagcaacctggtggcgggcattccggatc |
| | tttaccattagccgcgataacgcgaaaaacaccctgtatct | gctttagcggcagcggcagcggcaccgattttaccct |
| | gcagatgaacagcctgcgcgcggaagataccgcggtgtat | gaccattagccgcctggaaccggaagattttgcggtg |
| | tattgcgcgcgcctgccggattattggggccagggcaccct | tattattgccagcatcatagctggtatcattttaccttt |
| | ggtgaccgtgagcagc | ggcggcggcaccaaagtggaaattaaa |
| SEQ ID | 9 | 10 |
| NO: | | |
| Ab_42 | gaagtgcagctggtggaaagcggcggcggcctggtgcagc | gaaattgtgctgacccagagcccgggcaccctgagcc |
| | cgggcggcagcctgcgcctgagctgcgcggcgagcggcct | tgagcccgggcgaacgcgcgaccctgagctgccgcg |
| | gacctttagccgctatccgatgagctgggtgcgccaggcgc | cgagcagcagcgtgagcagcagctatctgcattggta |
| | cgggcaaaggcctggtgtgggtgagcgcgattaccagcag | tcagcagaaaccgggccaggcgccgcgcctgctgatt |
| | cggcggcagcacctattatagcgataccgtgaaaggccgc | tatagcaccagcaacctggtggcgggcattccggatc |
| | tttaccattagccgcgataacgcgaaagcgaccctgtatct | gctttagcggcagcggcagcggcaccgattttaccct |
| | gcagatgaacagcctgcgcgcggaagataccgcggtgtat | gaccattagccgcctggaaccggaagattttgcggtg |
| | tattgcgcgcgcctgccggattattggggccagggcaccct | tattattgccagcatcatagcggctatcattttaccttt |
| | ggtgaccgtgagcagc | ggcggcggcaccaaagtggaaattaaa |
| SEQ ID | 19 | 20 |
| NO: | | |
| Ab_65 | gaagtgcagctgctggaaagcggcggcggcctggtgcagc | gatattcagatgacccagagcccgagcagcctgagc |
| | cgggcggcagcctgcgcctgagctgcgcggcgagcggctt | gcgagcgtgggcgatcgcgtgaccattacctgcaaag |
| | tacctttagcagctatgcgatgagctgggtgcgccaggcgc | cgagccaggatgtgagcaccgcggtggcgtggtatc |
| | cgggcaaaggcctggaatgggtgagcaccattagcagcgg | agcagaaaccgggcaaagcgccgaaactgctgattt |
| | cggcagctataccagctatccggatagcgtgaaaggccgc | ataccgcgagctatcgcgaaaccggcgtgccgagcc |
| | tttaccattagccgcgataacagcaaaaacaccctgtatct | gctttagcggcagcggcagcggcaccgattttaccct |
| | gcagatgaacagcctgcgcgcggaagataccgcggtgtat | gaccattagcagcctgcagccggaagattttgcgacc |
| | tattgcgcgaaacaggtgtatgcgatgaactattggggcca | tattattgccagcagcattatagcaccccgtggacctt |
| | gggcaccctggtgaccgtgagcagc | tggcggcggcaccaaagtggaaattaaa |
| SEQ ID | 29 | 30 |
| NO: | | |
| Ab_86 | gaagtgcagctgctggaaagcggcggcggcctggtgcaga | gatattcagatgacccagagcccgagcagcctgagc |
| | ccggcggcagcctgcgcctgagctgcgcggcgagcggcttt | gcgagcgtgggcgatcgcgtgaccattacctgcaaaa |
| | acctttagcagctatgcgatgagctgggtgcgccaggcgcc | gcagccaggatgtgagcaccgcggtggcgtggtatc |
| | gggcaaaggcctggaatgggtgagcaccattagcagcggc | agcagaaaccgggcaaagcgccgaaactgctgattt |
| | ggcagctataccagctatccggatagcgtgaaaggccgctt | atagcgcgagctatcgctataccggcgtgccgagccg |
| | taccattagccgcgataacagcaaaaacaccctgtatctgc | ctttagcggcagcggcagcggcaccgattttaccctg |
| | agatgaacagcctgcgcgcggaagataccgcggtgtatta | agcattagcagcctgcagccggaagattttgcgacct |
| | ttgcgcgaaacaggattatgcgatgaactattggggccagg | attattgccagcagcattatattaccccgtggacctttg |
| | gcaccctggtgaccgtgagcagc | gcggcggcaccaaagtggaaattaaa |
| SEQ ID | 39 | 40 |
| NO: | | |
| Ab_06 | gaagtgcagctggtggaaagcggcggcggcctggtgcagc | gaaattgtgctgacccagagcccgggcaccctgagcc |
| | cgggcggcagcctgcgcctgagctgcgcggcgagcggcct | tgagcccgggcgaacgcgcgaccctgagctgccgcg |
| | gacctttagccgctatccgatgagctgggtgcgccaggcgc | cgagcagcagcgtgagcaaaagctatctgcattggta |
| | cgggcaaaggcctggtgtgggtgagcgcgattaccagcag | tcagcagaaaccgggccaggcgccgcgcctgctgatt |
| | cggcggcagcacctattatagcgataccgtgaaaggccgc | tatagcaccagcaacctggtggcgggcattccggatc |
| | tttaccattagccgcgataacgcgaaaaacaccctgtatct | gctttagcggcagcggcagcggcaccgattttaccct |
| | gcagatgaacagcctgcgcgcggaagataccgcggtgtat | gaccattagccgcctggaaccggaagattttgcggtg |
| | tattgcgcgcgcctgccggattattggggccagggcaccct | tattattgccagcatcatagcggcccgcatggcacctt |
| | ggtgaccgtgagcagc | tggcggcggcaccaaagtggaaattaaa |
| SEQ ID | 49 | 50 |
| NO: | | |
| Ab_43 | gaagtgcagctggtggaaagcggcggcggcctggtgcagc | gaaattgtgctgacccagagcccgggcaccctgagcc |
| | cgggcggcagcctgcgcctgagctgcaccgcgagcggcct | tgagcccgggcgaacgcgcgaccctgagctgccgcg |
| | gacctttagcaactatccgatgagctgggtgcgccaggcgc | cgagcagcagcgtgagcagcagctatctgcattggta |
| | cgggcaaaggcctggtgtgggtgagcgcgattaccagcag | tcagcagaaaccgggccaggcgccgcgcctgctgatt |
| | cggcggcagcacctattatagcgataccgtgaaaggccgc | tatagcaccagcaacctggtggcgggcattccggatc |
| | tttaccattagccgcgataacgcgaaaaacaccctgtatct | gctttagcggcagcggcagcggcaccgattttaccct |
| | gcagatgaacagcctgcgcgcggaagataccgcggtgtat | gaccattagccgcctggaaccggaagattttgcggtg |
| | tattgcgcgcgcctgccggattattggggccagggcaccct | tattattgccagcatgaaagcggctatcattttaccttt |
| | ggtgaccgtgagcagc | ggcggcggcaccaaagtggaaattaaa |
| SEQ ID | 59 | 60 |
| NO: | | |
| Ab_66 | gaagtgcagctgctggaaagcggcggcggcctggtgcagc | gatattcagatgacccagagcccgagcagcctgagc |
| | cgggcggcagcctgcgcctgagctgcgcggcgagcggctt | gcgagcgtgggcgatcgcgtgaccattacctgcaaag |
| | tagctttagcagctatgcgatgagctgggtgcgccaggcgc | cgagccaggatgtgagcaccgcggtggcgtggtatc |
| | cgggcaaaggcctggaatgggtgagcaccattagcagcgg | agcagaaaccgggcaaagcgccgaaactgctgattt |
| | cggcagctataccagctatccggatagcgtgaaaggccgc | atagcgcgagctatcgcctgaccggcgtgccgagccg |
| | tttaccattagccgcgataacagcaaaaacaccctgtatct | ctttagcggcagcggcagcggcaccgattttaccctg |
| | gcagatgaacagcctgcgcgcggaagataccgcggtgtat | accattagcagcctgcagccggaagattttgcgacct |
| | tattgcgcgaaacaggattatgcgatgaactattggggcca | attattgccagcagcattatagcaccccgtggaccttt |
| | gggcaccctggtgaccgtgagcagc | ggcggcggcaccaaagtggaaattaaa |
| SEQ ID | 69 | 70 |
| NO: | | |
| Ab_87 | gaagtgcagctgctggaaagcggcggcggcctggtgcagc | gatattcagatgacccagagcccgagcagcctgagc |
| | cgggcggcagcctgcgcctgagctgcgtggcgagcggcttt | gcgagcgtgggcgatcgcgtgaccattacctgcaaag |
| | acctttagcagctatgcgatgagctgggtgcgccaggcgcc | cgagccaggatgtgagcaccgcggtggcgtggtatc |
| | gggcaaaggcctggaatgggtgagcaccattagcagcggc | agcagaaaccgggcaaagcgccgaaactgctgattt |
| | ggcagctataccagctatccggatagcgtgaaaggccgctt | atagcgcgagctatcgcgaaaccggcgtgccgagcc |
| | taccattagccgcgataacagcaaaaacaccctgtatctgc | gctttagcggcagcggcagcggcaccgattttaccct |
| | agatgaacagcctgcgcgcggaagataccgcggtgtatta | gaccattagcagcctgcagccggaagattttgcgacc |
| | ttgcgcgaaacaggattatgcgatgaactattggggccagg | tattattgccagcagcattatagcaccccgtggacctt |
| | gcaccctggtgaccgtgagcagc | tggcggcggcaccaaagtggaaattaaa |
| SEQ ID | 79 | 80 |
| NO: | | |
| Ab_14 | gaagtgcagctggtggaaagcggcggcggcctggtgcagc | gaaattgtgctgacccagagcccgggcaccctgagcc |
| | cgggcggcagcctgcgcctgagctgcgcggcgagcggcct | tgagcccgggcgaacgcgcgaccctgagctgccgcg |
| | gacctttagccgctatccgatgagctgggtgcgccaggcgc | cgagcagcagcgtgagcagcagctatctgcattggta |
| | cgggcaaaggcctggtgtgggtgagcaccattaccagcag | tcagcagaaaccgggccaggcgccgcgcctgctgatt |
| | cggcggcggcacctattatagcgataccgtgaaaggccgc | tatagcaccagcaacctggtggcgggcattccggatc |
| | tttaccattagccgcgataacgcgaaaaacaccctgtatct | gctttagcggcagcggcagcggcaccgattataccct |
| | gcagatgaacagcctgcgcgcggaagataccgcggtgtat | gaccattagccgcctggaaccggaagattttgcggtg |
| | tattgcgcgcgcctgccggattattggggccagggcaccct | tattattgccagcatcatagcggctatccgtttaccttt |
| | ggtgaccgtgagcagc | ggcggcggcaccaaagtggaaattaaa |
| SEQ ID | 89 | 90 |
| NO: | | |
| Ab_44 | gaagtgcagctggtggaaagcggcggcggcctggtgcagc | gaaattgtgctgacccagagcccgggcaccctgagcc |
| | cgggcggcagcctgcgcctgagctgcgcggcgagcggcct | tgagcccgggcgaacgcgtgaccctgagctgccgcg |
| | gacctttggccgctatccgatgagctgggtgcgccaggcgc | cgagcagcagcctgagcagcagccatctgcattggta |
| | cgggcaaaggcctggtgtgggtgagcgcgattaccagcag | tcagcagaaaccgggccaggcgccgcgcctggtgat |
| | cggcggcagcacctattatagcgataccgtgaaaggccgc | ttatagcaccagcaacctggtggcgggcattccggat |
| | tttaccattagccgcgataacgcgaaaaccaccctgtatct | cgctttagcggcagcggcagcggcaccgattttaccc |
| | gcagatgaacagcctgcgcgcggaagataccgcgatttatt | tgaccattagccgcctggaaccggaagattttgcggt |
| | attgcgcgcgcctgccggattattggggccagggcaccctg | gtattattgccagcatcatagcggctatcattttacctt |
| | gtgaccgtgagcagc | tggcggcggcaccaaagtggaaattaaa |
| SEQ ID | 99 | 100 |
| NO: | | |
| Ab_69 | gaagtgcagctgctggaaagcggcggcggcctggtgcagc | gatattcagatgacccagagcccgagcagcctgagc |
| | cgggcggcagcctgcgcctgagctgcgcggcgagcggctt | gcgagcgtgggcgatcgcgtgaccattacctgcaaag |
| | tacctttagcagctatgcgatgagctgggtgcgccaggcgc | cgagccaggatgtgagcaccgcggtggcgtggtatc |
| | cgggcaaaggcctggaatgggtgagcaccattagcagcgg | agcagaaaccgggcaaagcgccgaaactgctgatgt |
| | cggcagctataccagctatccggatagcgtgaaaggccgc | atagcgcgagctatcgctataccggcgtgccgagccg |
| | tttaccattagccgcgataacagcaaaaacaccctgtatct | ctttagcggcagcggcagcggcaccgattttaccctg |
| | gcagatgaacagcctgcgcgcggaagataccgcggtgtat | accattattaacctgcagccggaagattttgcgaccta |
| | tattgcgcgaaacaggattatgcgatggattattggggcca | ttattgccagcagcattatagcaccccgtggacctttg |
| | gggcaccctggtgaccgtgagcagc | gcggcggcaccaaagtggaaattaaa |
| SEQ ID | 109 | 110 |
| NO: | | |
| Ab_15 | gaagtgcagctggtggaaagcggcggcggcctggtgcagc | gaaattgtgctgacccagagcccgggcaccctgagcc |
| | cgggcggcagcctgcgcctgagctgcgcggcgagcggcct | tgagcccgggcgaacgcgcgaccctgagctgccgcg |
| | gacctttagccgctatccgatgagctgggtgcgccaggcgc | cgagcagcagcgtgagcagcagctatctgcattggta |
| | cgggcaaaggcctggtgtgggtgagcgcgattaccagcag | tcagcagaaaccgggccaggcgccgcgcctgctgatt |
| | cggcggcagcacctattatagcgataccgtgaaaggccgc | tatgcgaccagcaacctggtggcgggcattccggatc |
| | tttaccattagccgcgataacgcgaaaaacaccctgtatct | gctttagcggcagcggcagcggcaccgattttaccct |
| | gcagatgaacagcctgcgcgcggaagataccgcggtgtat | gaccattagccgcctggaaccggaagattttgcggtg |
| | tattgcgcgcgcctgccggattattggggccagggcaccct | tattattgccagcattttagcggctatcattttacctt |
| | ggtgaccgtgagcagc | tggcggcggcaccaaagtggaaattaaa |
| SEQ ID | 119 | 120 |
| NO: | | |
| Ab_45 | gaagtgcagctggtggaaagcggcggcggcctggtgcagc | gaaattgtgctgacccagagcccgggcaccctgagcc |
| | cgggcggcagcctgcgcctgagctgcgcggcgagcggcct | tgagcccgggcgaacgcgcgaccctgagctgccgcg |
| | gacctttagccgctatccgatgagctgggtgcgccaggcgc | cgagcagcagcgtgagcagcagctatctgcattggta |
| | cgggcaaaggcctggtgtgggtgagcgcgattaccagcag | tcagcagaaaccgggccaggcgccgcgcctgctgatt |
| | cggcggcagcacctattatagcgataccgtgaaaggccgc | tatgcgaccagcaacctggtggcgggcattccggatc |
| | tttaccattagccgcgataacgcgaaaaacaccctgtatct | gctttagcggcagcggcagcggcaccgattttaccct |
| | gcagatgaacagcctgaccgcggaagataccgcggtgtat | gaccattagccgcctggaaccggaagattttgcggtg |
| | tattgcgcgcgcctgccggattattggggccagggcaccct | tattattgccagcatcatagcggctatcattttaccttt |
| | ggtgaccgtgagcagc | ggcggcggcaccaaagtggaaattaaa |
| SEQ ID | 129 | 130 |
| NO: | | |
| Ab_81 | gaagtgcagctgctggaaagcggcggcggcctggtgcagc | gatattcagatgacccagagcccgagcagcctgagc |
| | cgggcggcagcctgcgcctgagctgcgcggcgagcggctt | gcgagcgtgggcgatcgcgtgagcattacctgcaaa |
| | tacctttagcagctatgcgatgagctgggtgcgccaggcgc | gcgagccaggatgtgaccaccgcggtggcgtggtatc |
| | cgggcaaaggcctggaatgggtgagcaccattagcagcgg | agcagaaacgcggcaaagcgccgaaactgctgattt |
| | cggcagctataccagctatccggatagcgtgaaaggccgc | atagcgcgagctatcgctataccggcgtgccgagccg |
| | tttaccattagccgcgataacagcaaaaacaccctgtatct | ctttagcggcagcggcagcggcaccgattttaccctg |
| | gcagatgaacagcctgcgcgcggaagataccgcggtgtat | accattagcagcctgcagcgcgaagattttgcgacct |
| | tattgcgcgaaacaggattatgcgatgaaccagtggggcc | attattgccagcagcattatagcaccccgtggaccttt |
| | agggcaccctggtgaccgtgagcagc | ggcggcggcaccaaagtggaaattaaa |
| SEQ ID | 139 | 140 |
| NO: | | |
| Ab_38 | gaagtgcagctggtggaaagcggcggcggcctggtgcagc | gaaattgtgctgacccagagcccgggcaccctgagcc |
| | cgggcggcagcctgcgcctgagctgcgcggcgagcggcct | tgagcccgggcgaacgcgcgaccctgagctgccgcg |
| | gacctttagccgctatccgatgagctgggtgcgccaggcgc | cgagcagcagcgtgagcagcagccagctgcattggt |
| | cgggcaaaggcctggtgtgggtgagcgcgattaccagcag | atcagcagaaaccgggccaggcgccgcgcctgctga |
| | cggcggcagcacctattatagcgataccgtgaaaggccgc | tttatagcaccagcaacctggtgggggcattccgga |
| | tttaccattagccgcgataacgcgaaaaacaccctgtatct | tcgctttagcggcagcggcagcggcaccgattttacc |
| | gcagatgaacagcctgcgcgcggaagataccgcggtgtat | ctgaccattagccgcctggaaccggaagattttgcgg |
| | tattgcgcgcgcctgccggattattggggccagggcaccct | tgtattattgccagcatcatagcggcagccattttacct |
| | ggtgaccgtgagcagc | ttggcggcggcaccaaagtggaaattaaa |
| SEQ ID | 149 | 150 |
| NO: | | |
| Ab_46 | gaagtgcagctggtggaaagcggcggcgatctggtgcagc | gaaattgtgctgacccagagcccgggcaccctgagcc |
| | cgggcggcagcctgcgcctgagctgcgcgaccagcggcct | tgagcccgggcgaacgcgcgaccctgagctgccgcg |
| | gacctttagccgctatccgatgagctgggtgcgccaggcgc | cgagcagcagcgtgagcagcagctatctgcattggta |
| | cgggcaaaggcctggtgtgggtgagcgcgattaccagcag | tcagcagaaaccgggccaggcgccgcgcctgctgatt |
| | cggcggcagcacctattatagcgataccgtgaaaggccgc | tatagcaccagcaacctggtggcgggcattccggatc |
| | tttaccattagccgcgataacgcgaaaaacaccctgtatct | gctttagcggcagcggcagcggcaccgattttaccct |
| | gcagatgaacagcctgcgcgcggaagataccgcggtgtat | gaccattagccgcctggaaccggaagattttgcggtg |
| | tattgcgcgcgcctgccggattattggggccagggcaccct | tattattgccagcatagcagcggctatcatattaccttt |
| | ggtgaccgtgagcagc | ggcggcggcaccaaagtggaaattaaa |
| SEQ ID | 159 | 160 |
| NO: | | |
| Ab_82 | gaagtgcagctgctggaaagcggcggcggcctggtgcagc | gatattcagatgacccagagcccgagcagcctgagc |
| | cgggcggcagcctgcgcctgagctgcgcggcgagcggctt | gcgagcgtgggcgatcgcgtgaccattacctgcaaag |
| | tacctttagcagctatgcgatgagctgggtgcgccaggcgc | cgagccaggatgtgagcaccgcggtggcgtggtatc |
| | cgggcaaaggcctggaatgggtgagcaccattagcagcgg | agcagaaaccgggcaaagcgccgaaactgctgattt |
| | cggcagctataccagctatccggatagcgtgaaaggccgc | atagcgcgagctatcgctataccggcgtgccgagccg |
| | tttaccattagccgcgataacagcaaaaacaccctgtatct | ctttagcggcagcggcagcggcaccgattttaccctg |
| | gcagatgaacagcctgcgcgcggaagataccgcggtgtat | accattagcagcctgctgccggaagattttgcgacct |
| | tattgcgcgaaacaggattatgcgatgaactattggggcca | attattgccagcagcattatagcaccccgtggaccttt |
| | gggcaccctggtgaccgtgagcagc | ggcggcggcaccaaagtggaaattaaa |
| SEQ ID | 169 | 170 |
| NO: | | |
| Ab_39 | gaagtgcagctggtggaaagcggcggcggcctggtgcagc | gaaattgtgctgacccagagcccgggcacccagagc |
| | cgggcggcagcctgcgcctgagctgcgcggcgagcggctt | ctgagcccgggcgaacgcgcgaccctgagctgccgc |
| | taccgtgagcagctatggcatgagctgggtgcgccaggcg | gcgagcagcgcggtgagcggcagctatctgcattggt |
| | ccgggcaaaggcctggaatgggtgagcggcattcgcggca | atcagcagaaaccgggccaggcgccgcgcctgctga |
| | gcgatggcagcacctattatgcggatagcgtgaaaggccg | tttatagcaccagcaacctggtggcgggcattccgga |
| | ctttaccattagccgcgataacagcaaaaacaccctgtatc | tcgctttagcggcagcggcagcggcaccgattttacc |
| | tgcagatgaacagcctgcgcgcggaagataccgcggtgta | ctgaccattacccgcctggaaccggaagattttgcgg |
| | ttattgcgcgcgcctgccggattattggggccagggcaccc | tgtattattgccagcatcatagcgattatcattatacct |
| | tggtgaccgtgagcagc | ttggcggcggcaccaaagtggaaattaaa |
| SEQ ID | 179 | 180 |
| NO: | | |
| Ab_47 | gaagtgcagctggtggaaagcggcggcggcctggtgcagc | gaaattgtgctgacccagagcccgggcattctgagcc |
| | cgggcggcagcctgcgcctgagctgcgcggcgagcggcct | tgagcccgggcgaacgcgtgaccctgagctgccgcg |
| | gacctttagccgctatccgatgagctgggtgcgccaggcgc | cgagcagcagcgtgagcagcagctatctgcattggta |
| | cgggcaaaggcctggtgtgggtgagcagcattaccagcag | tcagcagaaaccgggccaggcgccgcgcctgctgatt |
| | cggcggcagcaccgattatagcgataccgtgaaaggccgc | tatagcaccagcaacctggtggcgggcattccggatc |
| | tttaccattagccgcgataacgcgaaaaacaccctgtatct | gctttagcggcagcggcagcggcaccgaatttaccct |
| | gcagatgaacagcctgcgcgcggaagataccgcggtgtat | gaccattagccgcctggaaccggaagattttgcggtg |
| | tattgcgcgcgcctgccggattattggggccagggcaccct | tattattgccagcatcatagcaccccgcattttaccttt |
| | ggtgaccgtgagcagc | ggcggcggcaccaaagtggaaattaaa |
| SEQ ID | 189 | 190 |
| NO: | | |
| Ab_83 | gaagtgcagctgctggaaagcggcggcggcctggtgcagc | gatattcagatgacccagagcccgagcagcctgagc |
| | cgggcggcagcctgcgcctgagctgcgcggcgagcggctt | gcgagcgtgggcgatcgcgtgaccattacctgcaaag |
| | tacctttagcagcaacgcgatgagctgggtgcgccaggcg | cgagccaggatgtgagcgatgcggtggcgtggtatca |
| | ccgggcaaaggcctggaatgggtgagcaccattagcagcg | gcagaccccgggcaaagcgccgaaactgctgatttat |
| | gcggcagctataccagctatccggatagcgtgaaaggccg | agcgcgagctatcgcccgaccggcgtgccgagccgct |
| | ctttaccattagccgcgataacagcaaaaacaccctgtatc | ttagcggcagcggcagcggcaccgattttaccctgac |
| | tgcagatgaacagcctgcgcgcggaagataccgcggtgta | cattagcagcctgcagccggaagattttgcgacctatt |
| | ttattgcgcgaaacaggattatgcgatgaactattggggcc | attgccagcagcattatagcaccccgtggacctttggc |
| | agggcaccctggtgaccgtgagcagc | ggcggcaccaaagtggaaattaaa |
| SEQ ID | 199 | 200 |
| NO: | | |
| Ab_40 | gaagtgcagctggtggaaagcggcggcggcctggtgcagc | gaaattgtgctgacccagagcccgggcaccctgagcc |
| | cgggcggcagcctgcgcctgagctgcgcggcgagcggcct | tgagcccgggcgaacgcgcgaccctgagctgccgcg |
| | gacctttagccgctatccgatgagctgggtgcgccaggcgc | cgagcagcattgtgagcagcagctatctgcattggta |
| | cgggcaaaggcctggtgtgggtgagcgcgattaccagcag | tcagcagaaaccgggccaggcgccgcgcctgctgatt |
| | cggcggcagcacctattatagcgataccgtgaaaggccgc | tatagcacctttaacctggtggcggatattccggatcg |
| | tttaccattagccgcgataacgcgaaaaacaccctgtatct | ctttagcggcagcggcagcggcaccgattttaccctg |
| | gcagatgaacagcctgcgcgcggaagataccgcgatttatt | accattagccgcctggaaccggaagattttgcggtgt |
| | attgcgtgcgcctgccggattattggggccagggcaccctg | attattgccagcatcatagcggctatccgtttacctttg |
| | gtgaccgtgagcagc | gcggcggcaccaaagtggaaattaaa |
| SEQ ID | 209 | 210 |
| NO: | | |
| Ab_56 | gaagtgcagctgctggaaagcggcggcggcctggtgcagc | gatattcagatgacccagagcccgagcagcctgagc |
| | cgggcggcagcctgcgcctgagctgcgcggcgagcggctt | gcgagcgtgggcgatcgcgtgaccattacctgcaaag |
| | tacctttagcagctatgcgatgagctgggtgcgccaggcgc | cgagccaggatgtgagcaccgcggtggcgtggtatc |
| | cgggcaaaggcctggaatgggtgagcaccattagcagcgg | agcagaaaccgggcaaagcgccgaaactgctgattt |
| | cggcagctataccagctatccggatagcgtgaaaggccgc | atagcgcgagctatcgctataccggcgtgccgagccg |
| | tttaccattagccgcgataacagcaaaaacaccctgtatct | ctttagcggcagcggcagcggcaccgattttaccctg |
| | gcagatgaacagcctgcgcgcggaagataccgcggtgtat | accattagcagcctgcagccggaagattttgcgacct |
| | tattgcgcgaaacaggattatgcgatgaactattggggcca | atcattgccagcagcattatagcaccccgtggaccttt |
| | gggcaccctggtgaccgtgagcagc | ggcggcggcaccaaagtggaaattaaa |
| SEQ ID | 219 | 220 |
| NO: | | |
| Ab_84 | gaagtgcagctgctggaaagcggcggcggcctggtgcagc | gatattcagatgacccagagcccgagcagcctgagc |
| | cgggcggcagcctgcgcctgagctgcgcggcgagcggctt | gcgagcgtgggcgatcgcgtgaccattacctgcaaag |
| | tacctttagcagctatgcgatgagctgggtgcgccaggcgc | cgagccaggatgtgagcaccgcggtggcgtggtatc |
| | cgggcaaaggcctggaatgggtgagcaccattagcagcgg | agcagaaaccgggcaaagcgccgaacctgctgattt |
| | cggcagctataccagctatccggatagcgtgaaaggccgc | atagcgcgagctatcgctataccggcgtgccgagccg |
| | tttaccattagccgcgataacagcaaaaacaccctgtatct | ctttagcggcagcggcagcggcaccgattttaccctg |
| | gcagatgaacagcctgcgcggcgaagataccgcgctgtat | accattagcagcctgcagccggaagattttgcgacct |
| | tattgcgcgcgccaggattatgcgatgaactattggggcca | attattgccagcagcattatagcaccccgtggaccttt |
| | gggcaccctggtgaccgtgagcagc | ggcggcggcaccaaagtggaaattaaa |
| SEQ ID | 229 | 230 |
| NO: | | |
| Ab_41 | gaagtgcagctggtggaaagcggcggcggcctggtgcagc | gaaattgtgctgacccagagcccgggcaccctgagcc |
| | cgggcggcagcctgcgcctgagctgcgcggcgagcggcct | tgagcccgggcgaacgcgcgaccctgagctgccgcg |
| | gacctttagccgctatccgatgagctgggtgcgccaggcgc | cgagcagcagcgtgagcattagctatctgcattggta |
| | cgggcaaaggcctggtgtgggtgagcgcgattaccagcag | tcaggtgaaaccgggccaggcgccgcgcctgctgatt |
| | cggcggcagcacctattatagcgataccgtgaaaggccgc | tatagcaccagcaacctggtggcgggcattccggatc |
| | tttaccattagccgcgataacgcgaaaaacaccctgtttct | gctttagcggcagcggcagcggcaccgattttaccct |
| | gcagatgaacagcctgcgcgcggaagataccgcggtgtatt | gaccattagccgcctggaaccggaagattttgcggtg |
| | attgcgcgcgcctgccggattattggggccagggcaccctg | tatttttgccagcatgaaagcggctatcattttaccttt |
| | gtgaccgtgagcagc | ggcggcggcaccaaagtggaaattaaa |
| SEQ ID | 239 | 240 |
| NO: | | |
| Ab_63 | gaagtgcagctgctggaaagcggcggcggcctggtgcagc | gatattcagatgacccagagcccgagcagcctgagc |
| | cgggccgcagcctgcgcctgagctgcgcggcgagcggcttt | gcgagcgtgggcgatcgcgtgaccattacctgcaaag |
| | acctttcatagctatgcgatgagctgggtgcgccaggcgcc | cgagccaggatgtgagcaccgcggtggcgtggtatc |
| | gggcaaaggcctggaatgggtgagcaccattagcagcggc | agcagaaaccgggcaaagcgccgaaactgctgatgt |
| | ggcagctataccagctatccggatagcgtgaaaggccgctt | atagcgcgagctatcgccgcaccggcgtgccgagcc |
| | taccattagccgcgataacagcaaaaacaccctgtatctgc | gctttagcggcagcggcagcggcaccgattttaccct |
| | agatgaacagcctgcgcgcggaagataccgcggtgtatta | gaccattagcagcctgcagccggaagatgtggcgac |
| | ttgcgcgaaacaggattatgcgatggatctgtggggccagg | ctattattgccagcagcattatagcaccccgtggacct |
| | gcaccctggtgaccgtgagcagc | ttggcggcggcaccaaagtggaaattaaa |
| SEQ ID | 249 | 250 |
| NO: | | |
| Ab_85 | gaagtgcagctgctggaaagcggcggcggcctggtgcagc | gatattcagatgacccagagcccgagcagcctgagc |
| | cgggcggcagcctgcgcctgagctgcgcggcgagcggctt | gcgagcgtgggcgatcgcgtgaccattacctgcaaag |
| | tacctttagcagctatgcgatgagctgggtgcgccaggcgc | cgagccaggatgtgagcaccgcggtggcgtggtatc |
| | cgggcaaaggcctggaatgggtgagcaccattagcagcgg | agcagaaaccgggcaaagcgccgaaactgctgattt |
| | cggcagctataccagctatccggatagcgtgaaaggccgc | atagcgcgagctatcgctataccggcgtgccgagccg |
| | tttaccattagccgcgataacagcaaaaacaccctgtatct | ctttagcggcagcggcagcggcaccgattttaccctg |
| | gcagatgaacagcctgcgcgcggaagataccgcggtgtat | accattagccgcctgcagccggaagattttgcgacct |
| | tattgcgcgaaacaggatgcgatgaactattggggccagg | attattgccagcagcattatagcaccccgtggaccttt |
| | gcaccctggtgaccgtgagcagc | ggcggcggcaccaaagtggaaattaaa |
| SEQ ID | 259 | 260 |
| NO: | | |

### Fc Domain Sequences

The term variable domain and variable region are used interchangeably and refer to the portions of the light and heavy chains of an antibody that include the complementarity determining regions and framework regions (FRs).

Table 4 provides amino acid sequences for the Fc region or domain.

| | | |
|---|---|---|
| Fc 1 | ASTKGPSVFPLAPSSKS | GCATCCACCAAGGGCCCATCGGTCTTCCCCCTGGCACCCTCCTCCAA |
| | TSGGTAALGCLVKDYFP | GAGCACCTCTGGGGGCACAGCGGCCCTGGGCTGCCTGGTCAAGGAC |
| | EPVTVSWNSGALTSGV | TACTTCCCCGAACCGGTGACGGTGTCGTGGAACTCAGGCGCCCTGA |
| | HTFPAVLQSSGLYSLSS | CCAGCGGCGTGCACACCTTCCCGGCTGTCCTACAGTCCTCAGGACTC |
| | VVTVPSSSLGTQTYICN | TACTCCCTCAGCAGCGTGGTGACCGTGCCCTCCAGCAGCTTGGGCAC |
| | VNHKPSNTKVDKKVEP | CCAGACCTACATCTGCAACGTGAATCACAAGCCCAGCAACACCAAG |
| | KSCDKTHTCPPCPAPEL | GTGGACAAGAAAGTTGAGCCCAAATCTTGTGACAAAACTCACACAT |
| | LGGPSVFLFPPKPKDTL | GCCCACCGTGCCCAGCACCTGAACTCCTGGGGGGACCGTCAGTCTTC |
| | MISRTPEVTCVVVDVS | CTCTTCCCCCCAAAACCCAAGGACACCCTCATGATCTCCCGGACCCCT |
| | HEDPEVKFNWYVDGV | GAGGTCACATGCGTGGTGGTGGACGTGAGCCACGAAGACCCTGAG |
| | EVHNAKTKPREEQYNS | GTCAAGTTCAACTGGTACGTGGACGGCGTGGAGGTGCATAATGCCA |
| | TYRVVSVLTVLHQDWL | AGACAAAGCCGCGGGAGGAGCAGTACAACAGCACGTACCGTGTGG |
| | NGKEYKCKVSNKALPA | TCAGCGTCCTCACCGTCCTGCACCAGGACTGGCTGAATGGCAAGGA |
| | PIEKTISKAKGQPREPQ | GTACAAGTGCAAGGTCTCCAACAAAGCCCTCCCAGCCCCCATCGAGA |
| | VYTLPPSRDELTKNQVS | AAACCATCTCCAAAGCCAAAGGGCAGCCCCGAGAACCACAGGTGTA |
| | LTCLVKGFYPSDIAVEW | CACCCTGCCCCCATCCCGGGATGAGCTGACCAAGAACCAGGTCAGC |
| | ESNGQPENNYKTTPPV | CTGACCTGCCTGGTCAAAGGCTTCTATCCCAGCGACATCGCCGTGGA |
| | LDSDGSFFLYSKLTVDKS | GTGGGAGAGCAATGGGCAGCCGGAGAACAACTACAAGACCACGCC |
| | RWQQGNVFSCSVMHE | TCCCGTGCTGGACTCCGACGGCTCCTTCTTCCTCTATAGCAAGCTCAC |
| | ALHNHYTQKSLSLSPGK | CGTGGACAAGAGCAGGTGGCAGCAGGGGAACGTCTTCTCATGCTCC |
| | | GTGATGCATGAGGCTCTGCACAACCACTACACGCAGAAGAGCCTCT |
| | | CCCTGTCTCCGGGTAAA |
| SEQ | 261 | 262 |
| ID | | |
| NO: | | |
| Fc 2 | ASTKGPSVFPLAPCSRS | GCCAGTACAAAAGGACCTAGCGTGTTCCCTTTGGCTCCCTGTAGTCG |
| | TSESTAALGCLVKDYFP | TTCAACCTCTGAAAGCACAGCAGCGCTGGGGTGTTTGGTCAAGGAC |
| | EPVTVSWNSGALTSGV | TATTTTCCCGAACCCGTGACCGTCAGCTGGAATTCTGGGGCGTTGAC |
| | HTFPAVLQSSGLYSLSS | CTCTGGAGTTCATACATTTCCCGCTGTGCTCCAAAGTTCCGGCTTGTA |
| | VVTVPSSSLGTKTYTCN | TTCCCTTAGTAGCGTTGTTACCGTTCCATCATCCAGCTTGGGCACCAA |
| | VDHKPSNTKVDKRVES | AACTTATACCTGTAATGTTGATCATAAACCCTCAAATACAAAGGTCG |
| | KYGPPCPPCPAPEFLGG | ATAAGAGGGTCGAAAGCAAATACGGGCCCCCTTGTCCGCCCTGTCC |
| | PSVFLFPPKPKDTLMIS | AGCGCCCGAGTTTCTGGGCGGGCCTTCTGTGTTCTTGTTCCCGCCAA |
| | RTPEVTCVVVDVSQED | AACCCAAAGATACTCTCATGATTAGCCGAACTCCCGAGGTGACATGC |
| | PEVQFNWYVDGVEVH | GTCGTCGTTGATGTCTCTCAAGAAGATCCTGAAGTGCAATTTAATTG |
| | NAKTKPREEQFNSTYR | GTATGTTGATGGCGTGGAAGTACACAATGCTAAGACCAAGCCCCGC |
| | VVSVLTVLHQDWLNGK | GAGGAACAATTTAATAGCACTTACCGTGTGGTGAGCGTTCTCACTGT |
| | EYKCKVSNKGLPSSIEKT | CCTGCATCAAGACTGGTTGAATGGTAAAGAATATAAATGCAAAGTG |
| | ISKAKGQPREPQVYTLP | TCCAATAAAGGACTGCCCAGCAGCATCGAAAAGACCATATCCAAAG |
| | PSQEEMTKNQVSLTCL | CTAAGGGGCAACCGCGAGAACCTCAAGTTTATACGCTCCCACCATCC |
| | VKGFYPSDIAVEWESN | CAAGAAGAAATGACAAAGAACCAAGTGTCCCTTACTTGCTTGGTGA |
| | GQPENNYKTTPPVLDS | AGGGCTTTTACCCTTCAGATATAGCAGTAGAATGGGAAAGCAATGG |
| | DGSFFLYSRLTVDKSRW | CCAACCTGAAAACAATTATAAAACAACTCCCCCAGTACTGGATAGTG |
| | QEGNVFSCSVMHEALH | ATGGCTCTTTCTTCCTTTATAGCCGTCTTACAGTTGACAAGAGCCGCT |
| | NHYTQKSLSLSLG | GGCAAGAAGGCAATGTGTTCTCTTGTTCAGTCATGCACGAAGCGCTC |
| | | CACAATCATTATACTCAGAAGAGTTTGTCACTCAGTTTGGGG |
| SEQ | 263 | 264 |
| ID | | |
| NO: | | |
| Fc 3 | ASTKGPSVFPLAPCSRS | GCCAGTACAAAAGGACCTAGCGTGTTCCCTTTGGCTCCCTGTAGTCG |
| | TSESTAALGCLVKDYFP | TTCAACCTCTGAAAGCACAGCAGCGCTGGGGTGTTTGGTCAAGGAC |
| | EPVTVSWNSGALTSGV | TATTTTCCCGAACCCGTGACCGTCAGCTGGAATTCTGGGGCGTTGAC |
| | HTFPAVLQSSGLYSLSS | CTCTGGAGTTCATACATTTCCCGCTGTGCTCCAAAGTTCCGGCTTGTA |
| | VVTVPSSSLGTKTYTCN | TTCCCTTAGTAGCGTTGTTACCGTTCCATCATCCAGCTTGGGCACCAA |
| | VDHKPSNTKVDKRVES | AACTTATACCTGTAATGTTGATCATAAACCCTCAAATACAAAGGTCG |
| | KYGPPCPPCPAPEFLGG | ATAAGAGGGTCGAAAGCAAATACGGGCCCCCTTGTCCGCCCTGTCC |
| | PSVFLFPPKPKDTLMIS | AGCGCCCGAGTTTCTGGGCGGGCCTTCTGTGTTCTTGTTCCCGCCAA |
| | RTPEVTCVVVDVSQED | AACCCAAAGATACTCTCATGATTAGCCGAACTCCCGAGGTGACATGC |
| | PEVQFNWYVDGVEVH | GTCGTCGTTGATGTCTCTCAAGAAGATCCTGAAGTGCAATTTAATTG |
| | NAKTKPREEQFNSTYR | GTATGTTGATGGCGTGGAAGTACACAATGCTAAGACCAAGCCCCGC |
| | VVSVLTVLHQDWLNGK | GAGGAACAATTTAATAGCACTTACCGTGTGGTGAGCGTTCTCACTGT |
| | EYKCKVSNKGLPSSIEKT | CCTGCATCAAGACTGGTTGAATGGTAAAGAATATAAATGCAAAGTG |
| | ISKAKGQPREPQVYTLP | TCCAATAAAGGACTGCCCAGCAGCATCGAAAAGACCATATCCAAAG |
| | PSQEEMTKNQVSLTCL | CTAAGGGGCAACCGCGAGAACCTCAAGTTTATACGCTCCCACCATCC |
| | VKGFYPSDIAVEWESN | CAAGAAGAAATGACAAAGAACCAAGTGTCCCTTACTTGCTTGGTGA |
| | GQPENNYKTTPPVLDS | AGGGCTTTTACCCTTCAGATATAGCAGTAGAATGGGAAAGCAATGG |
| | DGSFFLYSRLTVDKSRW | CCAACCTGAAAACAATTATAAAACAACTCCCCCAGTACTGGATAGTG |
| | QEGNVFSCSVLHEALH | ATGGCTCTTTCTTCCTTTATAGCCGTCTTACAGTTGACAAGAGCCGCT |
| | SHYTQKSLSLSLG | GGCAAGAAGGCAATGTGTTCTCTTGTTCAGTCCTGCACGAAGCGCTC |
| | | CACTCTCATTATACTCAGAAGAGTTTGTCACTCAGTTTGGGG |
| SEQ | 265 | 266 |
| ID | | |
| NO: | | |

DIO Mouse Study. Male C57BL/6J mice with diet-induced obesity (DIO) were used in this study. Obesity was induced over 12-16 weeks through ad libitum access to a high-fat diet (HFD, 60% kcal from fat). Water was provided ad libitum throughout the study, and mice continued on HFD for the duration of the study.

Mice were randomized into four treatment groups: Group 1: Ab15_mIgG1 vehicle+semaglutide (SEMA), Group 2: Ab15_mIgG1 1 mg/kg+SEMA, Group 3: Ab15_mIgG1 3 mg/kg+SEMA, and Group 4: Ab15_mIgG1 10 mg/kg+SEMA. Ab15_mIgG1 or vehicle was administered twice weekly via subcutaneous (SC) injection at a dose volume of 5 mL/kg for the duration of the study (eight weeks). SEMA was administered SC once daily at 30 nmol/kg for the first four weeks, followed by vehicle administration in weeks 5-8.

Dual-energy X-ray absorptiometry (DEXA) scans were performed at baseline (week 0) and at weeks 2, 4, 6, and 8 to assess body composition. For each DEXA scan, mice were anesthetized with ketamine/xylazine. Body weight and food intake were recorded twice weekly for all animals throughout the study.

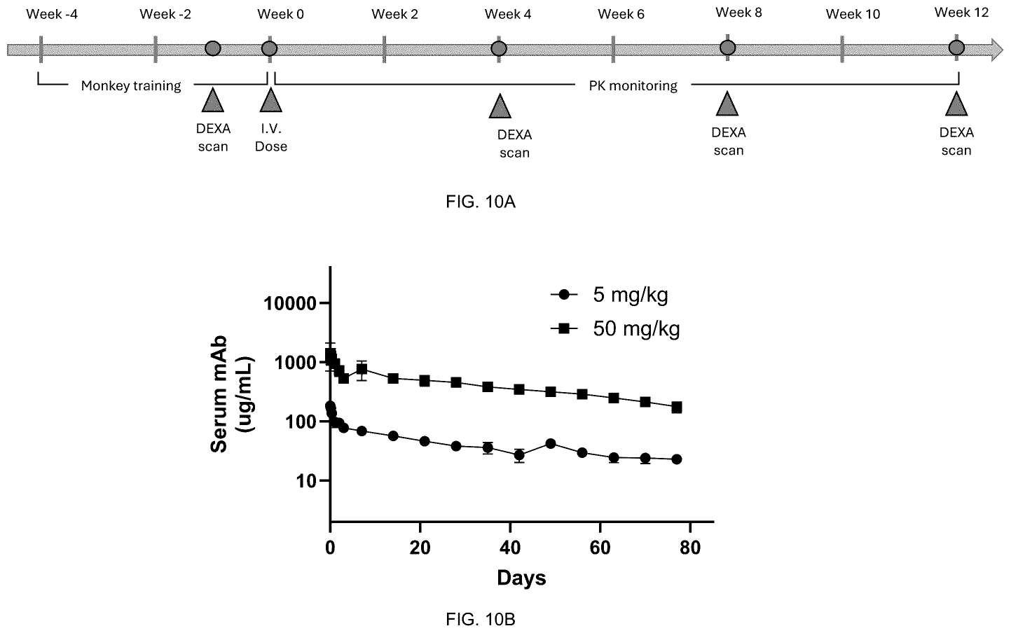

FIG. 9A shows a DIO mouse study design to test Ab_15 with semaglutide. FIG. 9B is a graph that shows the baseline corrected percent change in lean mass measured by DEXA scan. Obese NHP Study. Male diet-induced obese (DIO) cynomolgus monkeys (Macaca fascicularis), aged 16-21 years, with a body weight >7.5 kg and BMI >35 kg/m², were used in this study. Animals were housed under standard conditions with ad libitum access to water and a controlled diet. Food and water intake were monitored throughout the study, and daily cage-side clinical observations were performed.

Animals were randomized into two treatment groups (n=3 per group) and received a single intravenous (IV) dose of Ab_15 Fc_3. Group 1 received 5 mg/kg, and Group 2 received 50 mg/kg. The dosing volume was adjusted based on individual body weight.

Serum samples were collected at predefined time points following dosing. At each time point, 4 mL of whole blood was collected from a peripheral vein and transferred into serum separation tubes (SST). Samples were centrifuged at 2500×g for 10 minutes at 4° C., and the resulting serum was aliquoted into three 400 μL fractions and stored at −80° C. until analysis.

Whole-body composition was assessed at baseline, week 4, week 8, and week 12 using dual-energy X-ray absorptiometry (DEXA). Scans were performed with animals under sedation with ketamine hydrochloride (10 mg/kg IM).

Serum concentrations of Ab_15 Fc_3 were determined using a Human IgG4 ELISA kit (Cayman Chemical). Monkey serum samples were diluted 1:10 in assay buffer, and a standard curve was generated to quantify antibody concentrations at each time point. Antibody half-life was calculated based on a linear elimination model, using the serum concentration data obtained from the ELISA assay.

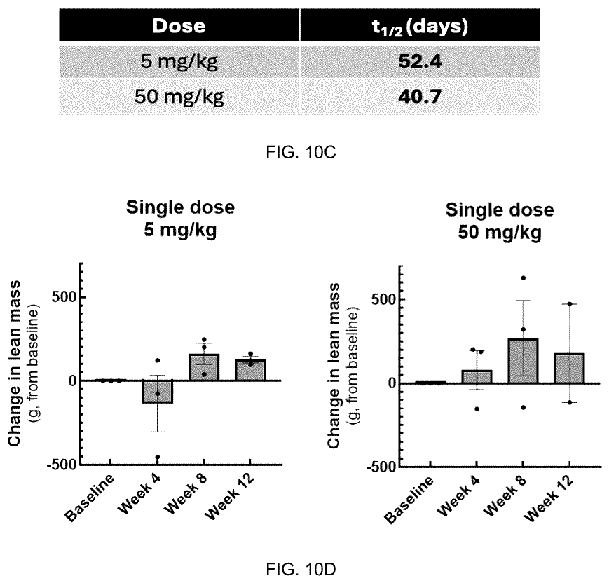

FIG. 10A shows an Obese NHP study design to test Ab_15 Fc_3, and FIG. 10B is a graph that shows serum antibody concentration levels over time, after single I.V. dose at 2 levels. FIG. 10C is a table with the calculated linear elimination half-life, and FIG. 10D is a graph that shows a change in lean mass from baseline based on DEXA scan analysis. In week 12 data at 50 mg/kg, one NHP was excluded from analysis due to diagnosed giardia infection.

### Administration of Therapeutic Anti-Myostatin Antibodies

The in vivo administration of the therapeutic anti-myostatin antibodies described herein may be carried out intravenously, intramuscularly, subcutaneously, topically, orally, transdermally, intraperitoneally, intraorbitally, intrathecally, intraventricularly, intranasally, transmucosally, through implantation, or through inhalation. Intravenous administration may be carried out via injection or infusion. In some embodiments, the therapeutic anti-myostatin antibodies of the disclosure are administered intravenously. In some embodiments, the therapeutic anti-myostatin antibodies of the disclosure are administered subcutaneously. Administration of the therapeutic anti-myostatin antibodies may be performed with any suitable excipients, carriers, or other agents to provide suitable or improved tolerance, transfer, delivery, and the like.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An anti-myostatin antibody or antigen binding domain thereof, wherein the antibody or antigen binding domain thereof comprises:
a. a heavy chain variable domain (VH) complementarity determining region (CDR) 1, VH CDR2, and VH CDR3 comprising an amino acid sequence of any one of the following SEQ ID NOS: 3, 4, 5; 13, 14, 15; 23, 24, 25; 33, 34, 35; 43, 44, 45; 53, 54, 55; 63, 64, 65; 73, 74, 75; 83, 84, 85; 93, 94, 95; 103, 104, 105; 113, 114, 115; 123, 124, 125; 133, 134, 135; 143, 144, 145; 153, 154, 155; 163, 164, 165; 173, 174, 175; 183, 184, 185; 193, 194, 195; 203, 204, 205; 213, 214, 215; 223, 224, 225; 233, 234, 235; 243, 244, 245; or 253, 254, 255, respectively; and
b. a light chain variable domain (VL) CDR1, VL CDR2, and VL CDR3 comprising the amino acid sequence of any one of the following SEQ ID NOS: 6, 7, 8; 16, 17, 18; 26, 27, 28; 36, 37, 38; 46, 47, 48; 56, 57, 58; 66, 67, 68; 76, 77, 78; 86, 87, 88; 96, 97, 98; 106, 107, 108; 116, 117, 118; 126, 127, 128; 136, 137, 138; 146, 147, 148; 156, 157, 158; 166, 167, 168; 176, 177, 178; 186, 187, 188; 196, 197, 198; 206, 207, 208; 216, 217, 218; 226, 227, 228; 236, 237, 238; 246, 247, 248; or 256, 257, 258, respectively; or
c. a heavy chain amino acid sequence having at least 95, 96, 97, 98, 99, or 100% sequence identity to any one of the following SEQ ID NOS: 1, 11, 21, 31, 41, 51, 61, 71, 81, 91, 101, 111, 121, 131, 141, 151, 161, 171, 181, 191, 201, 211, 221, 231, 241, or 251, respectively; and
d. a light chain amino acid sequence having at least 95, 96, 97, 98, 99, or 100% sequence identity to any one of the following SEQ ID NOS: 2, 12, 22, 32, 42, 52, 62, 72, 82, 92, 102, 112, 122, 132, 142, 152, 162, 172, 182, 192, 202, 212, 222, 232, 242, or 252, respectively.

2. The antibody or antigen binding domain of claim 1, wherein the antibody or antigen binding domain comprises:
a. a VH comprising the amino acid sequence of any one of the following SEQ ID NOS: 1, 11, 21, 31, 41, 51, 61, 71, 81, 91, 101, 111, 121, 131, 141, 151, 161, 171, 181, 191, 201, 211, 221, 231, 241, or 251, and
b. a VL comprising the amino acid sequence of any one of the following SEQ ID NOS: 2, 12, 22, 32, 42, 52, 62, 72, 82, 92, 102, 112, 122, 132, 142, 152, 162, 172, 182, 192, 202, 212, 222, 232, 242, or 252.

3. The antibody or antigen binding domain of claim 1, wherein the antibody is a monoclonal antibody.

4. The antibody or antigen binding domain of claim 1, wherein the antibody is a full-length antibody.

5. The antibody or antigen binding domain of claim 1, wherein the antibody is an antibody domain or single chain antigen binding domain of the heavy chain selected from SEQ ID NOS: 9, 19, 29, 39, 49, 59, 69, 79, 89, 99, 109, 119, 129, 139, 149, 159, 169, 179, 189, 199, 209, 219, 229, 239, 249, or 259; and the light chain selected from SEQ ID NOS: 10, 20, 30, 40, 50, 60, 70, 80, 90, 100, 110, 120, 130, 140, 150, 160, 170, 180, 190, 200, 210, 220, 230, 240, 250, or 260.

6. The antibody or antigen binding domain of claim 5, wherein the antigen binding domain is fused to an Fc domain of any one of the following: human IgG1, human IgG2, human IgG3, and human IgG4.

7. The antibody or antigen binding domain of claim 1, wherein the antibody comprises an Fc domain having an amino acid sequence selected from SEQ ID NOS: 261, 263, or 265.

8. The antibody or antigen binding domain of claim 1, wherein the antibody or antigen binding domain heavy chain amino acid sequence is encoded by a nucleic acid having at least 90, 91, 92, 93, 94, 95, 96, 97, 98, 99, or 100% sequence identity to any one of the following SEQ ID NOS: 9, 19, 29, 39, 49, 59, 69, 79, 89, 99, 109, 119, 129, 139, 149, 159, 169, 179, 189, 199, 209, 219, 229, 239, 249, or 259; and
the antibody or antigen binding domain light chain amino acid sequence is encoded by a nucleic acid having at least 90, 91, 92, 93, 94, 95, 96, 97, 98, 99, or 100% sequence identity to any one of the following SEQ ID NOS: 10, 20, 30, 40, 50, 60, 70, 80, 90, 100, 110, 120, 130, 140, 150, 160, 170, 180, 190, 200, 210, 220, 230, 240, 250, or 260.

9. A method of treating a muscle condition or metabolic disease in a subject in need thereof, comprising administering to the subject a therapeutically effective amount of the antibody or antigen binding domain of claim 1.

10. The method of claim 9, wherein the muscle condition is selected from myopathy, muscular atrophy, muscular dystrophy, and nerve injury, sarcopenia, cachexia, Parkinson's disease, osteoporosis, osteoarthritis, osteopenia, muscle injury, muscle wasting from disuse, immobilization, bed rest, injury, medical treatments, or surgical interventions.

11. The method of claim 9, wherein the muscle condition is associated with spinal muscular atrophy (SMA), amyotrophic lateral sclerosis (ALS), or myasthenia gravis.

12. The method of claim 10, wherein the nerve injury comprises partial denervation of neurons that innervate muscle, or impaired signaling between a motor neuron and a target muscle.

13. The method of claim 9, wherein the metabolic disease selected from the group consisting of type I diabetes, type II diabetes, obesity, metabolic syndrome/pre-diabetes, cardiovascular disease, non-alcoholic steatohepatitis (NASH), spinal cord injury (SCI), a hypo-metabolic state, double diabetes, Cushings disease, and an obesity syndrome.

14. The method of claim 9, wherein the antibody or antigen binding domain is provided in an amount sufficient to treat at least one of: treatment of a muscle wasting disease, increasing mass of muscle tissue, increasing strength of muscle tissue, enhance force generation, or prevent muscle loss, wherein the subject has, or is at risk of, at least one of: developing myopathy, muscle atrophy, or a metabolic disorder.

15. The method of claim 9, wherein the subject is human.

16. The method of claim 9, wherein the antibody or antigen binding domain is provided in an amount effective to cause one or more of the following in the subject: (a) an increase in mass and/or function of a muscle tissue in the subject; (b) an increase in a metabolic rate of the subject; (c) an increase in insulin sensitivity of the subject; (d) an increase in a level of brown adipose tissue in the subject; (e) an increase in a level of beige adipose tissue in the subject; (f) a decrease in a level of white adipose tissue in the subject; (g) a decrease in a level of visceral adipose tissue in the subject; (h) a decrease in ratio of adipose-to-muscle tissue in the subject; (i) an increase in glucose uptake by a brown adipose tissue, a beige adipose tissue, or a muscle tissue in the subject; (j) a decrease in glucose uptake by a white adipose tissue or a liver tissue; (k) a decrease in muscle catabolism of protein and/or muscle release of amino acids in the subject; (l) an increase in insulin dependent glycemic control in the subject; (m) a decrease in intramuscular fat infiltration in the subject; (n) an improvement in quality of life, as assessed by a standardized quality of life test; (o) prevention of muscle loss or atrophy in the subject; or (p) prevention of developing a metabolic dysregulation associated with muscle dysfunction in the subject, wherein the subject is a human subject that benefits from reduced myostatin signaling.

17. A polynucleotide encoding an anti-myostatin antibody or antigen binding domain thereof comprising:
a heavy chain variable domain (VH) complementarity determining region (CDR) 1, VH CDR2, and VH CDR3 comprising an amino acid sequence of any one of the following SEQ ID NOS: 3, 4, 5; 13, 14, 15; 23, 24, 25; 33, 34, 35; 43, 44, 45; 53, 54, 55; 63, 64, 65; 73, 74, 75; 83, 84, 85; 93, 94, 95; 103, 104, 105; 113, 114, 115; 123, 124, 125; 133, 134, 135; 143, 144, 145; 153, 154, 155; 163, 164, 165; 173, 174, 175; 183, 184, 185; 193, 194, 195; 203, 204, 205; 213, 214, 215; 223, 224, 225; 233, 234, 235; 243, 244, 245; or 253, 254, 255, respectively; and a light chain variable domain (VL) CDR1, VL CDR2, and VL CDR3 comprising the amino acid sequence of any one of the following SEQ ID NOS: 6, 7, 8; 16, 17, 18; 26, 27, 28; 36, 37, 38; 46, 47, 48; 56, 57, 58; 66, 67, 68; 76, 77, 78; 86, 87, 88; 96, 97, 98; 106, 107, 108; 116, 117, 118; 126, 127, 128; 136, 137, 138; 146, 147, 148; 156, 157, 158; 166, 167, 168; 176, 177, 178; 186, 187, 188; 196, 197, 198; 206, 207, 208; 216, 217, 218; 226, 227, 228; 236, 237, 238; 246, 247, 248; or 256, 257, 258, respectively; or
a heavy chain amino acid sequence is encoded by a nucleic acid having at least 90, 91, 92, 93, 94, 95, 96, 97, 98, 99, or 100% sequence identity to any one of the following SEQ ID NOS: 9, 19, 29, 39, 49, 59, 69, 79, 89, 99, 109, 119, 129, 139, 149, 159, 169, 179, 189, 199, 209, 219, 229, 239, 249, or 259; and
a light chain amino acid sequence is encoded by a nucleic acid having at least 90, 91, 92, 93, 94, 95, 96, 97, 98, 99, or 100% sequence identity to any one of the following SEQ ID NOS: 10, 20, 30, 40, 50, 60, 70, 80, 90, 100, 110, 120, 130, 140, 150, 160, 170, 180, 190, 200, 210, 220, 230, 240, 250, or 260.

18. The polynucleotide of claim 17, wherein the antibody is a monoclonal, bispecific, multivalent, multi-specific, diabody, chimeric, scFv antibody, domains or fragments thereof.

19. The polynucleotide of claim 17, wherein the antigen binding domain is fused to an Fc domain of any one of the following: human IgG1, human IgG2, human IgG3, and human IgG4.

20. The polynucleotide of claim 17, wherein a nucleic acid sequence is optimized for expression in a human, mouse, rat, hamster, bacterial, fungal, insect, or plant cell.

21. The polynucleotide of claim 17, further comprising a vector comprising the polynucleotide.

22. The polynucleotide of claim 21, further comprising a host cell comprising the vector.

23. A method of making an anti-myostatin antibody or binding domain thereof comprising expression in a host cell a polynucleotide that expresses:
a heavy chain variable domain (VH) complementarity determining region (CDR) 1, VH CDR2, and VH CDR3 comprising an amino acid sequence of any one of the following SEQ ID NOS: 3, 4, 5; 13, 14, 15; 23, 24, 25; 33, 34, 35; 43, 44, 45; 53, 54, 55; 63, 64, 65; 73, 74, 75; 83, 84, 85; 93, 94, 95; 103, 104, 105; 113, 114, 115; 123, 124, 125; 133, 134, 135; 143, 144, 145; 153, 154, 155; 163, 164, 165; 173, 174, 175; 183, 184, 185; 193, 194, 195; 203, 204, 205; 213, 214, 215; 223, 224, 225; 233, 234, 235; 243, 244, 245; or 253, 254, 255, respectively; and a light chain variable domain (VL) CDR1, VL CDR2, and VL CDR3 comprising the amino acid sequence of any one of the following SEQ ID NOS: 6, 7, 8; 16, 17, 18; 26, 27, 28; 36, 37, 38; 46, 47, 48; 56, 57, 58; 66, 67, 68; 76, 77, 78; 86, 87, 88; 96, 97, 98; 106, 107, 108; 116, 117, 118; 126, 127, 128; 136, 137, 138; 146, 147, 148; 156, 157, 158; 166, 167, 168; 176, 177, 178; 186, 187, 188; 196, 197, 198; 206, 207, 208; 216, 217, 218; 226, 227, 228; 236, 237, 238; 246, 247, 248; or 256, 257, 258, respectively; or
a heavy chain amino acid sequence is encoded by a nucleic acid having at least 90, 91, 92, 93, 94, 95, 96, 97, 98, 99, or 100% sequence identity to any one of the following SEQ ID NOS: 9, 19, 29, 39, 49, 59, 69, 79, 89, 99, 109, 119, 129, 139, 149, 159, 169, 179, 189, 199, 209, 219, 229, 239, 249, or 259; and
a light chain amino acid sequence is encoded by a nucleic acid having at least 90, 91, 92, 93, 94, 95, 96, 97, 98, 99, or 100% sequence identity to any one of the following SEQ ID NOS: 10, 20, 30, 40, 50, 60, 70, 80, 90, 100, 110, 120, 130, 140, 150, 160, 170, 180, 190, 200, 210, 220, 230, 240, 250, or 260.


SEQUENCE LISTING (267 sequences)

SEQ ID NO: 1 - protein, 113 aa, Homo sapiens
EVQLVESGGGLVQPGGSLRLSCAASGLTFSRYPMSWVRQAPGKGLVWVSAITSSGGSTYYSDTVKGRFTISRDNAKNTLYLQMNSLRAEDTAVYYCARLPDYWGQGTLVTVSS

SEQ ID NO: 2 - protein, 108 aa, Homo sapiens
EIVLTQSPGTLSLSPGERATLSCRASSSVSSSYLHWYQQKPGQAPRLLIYSTSNLVAGIPDRFSGSGSGTDFTLTISRLEPEDFAVYYCQHHSWYHFTFGGGTKVEIK

SEQ ID NO: 3 - protein, 8 aa, Homo sapiens
GLTFSRYP

SEQ ID NO: 4 - protein, 8 aa, Homo sapiens
ITSSGGST

SEQ ID NO: 5 - protein, 6 aa, Homo sapiens
ARLPDY

SEQ ID NO: 6 - protein, 7 aa, Homo sapiens
SSVSSSY

SEQ ID NO: 7 - skipped

SEQ ID NO: 8 - protein, 9 aa, Homo sapiens
QHHSWYHFT

SEQ ID NO: 9 - DNA, 339 bp, Homo sapiens
  source 1..339: mol_type = unassigned DNA
gaagtgcagctggtggaaagcggcggcggcctggtgcagccgggcggcagcctgcgcctgagctgcgcggcgagcggcctgacctttagccgctatccgatgagctgggtgcgccaggcgccgggcaaaggcctggtgtgggtgagcgcgattaccagcagcggcggcagcacctattatagcgataccgtgaaaggccgctttaccattagccgcgataacgcgaaaaacaccctgtatctgcagatgaacagcctgcgcgcggaagataccgcggtgtattattgcgcgcgcctgccggattattggggccagggcaccctggtgaccgtgagcagc

SEQ ID NO: 10 - DNA, 324 bp, Homo sapiens
  source 1..324: mol_type = unassigned DNA
gaaattgtgctgacccagagcccgggcaccctgagcctgagcccgggcgaacgcgcgaccctgagctgccgcgcgagcagcagcgtgagcagcagctatctgcattggtatcagcagaaaccgggccaggcgccgcgcctgctgatttatagcaccagcaacctggtggcgggcattccggatcgctttagcggcagcggcagcggcaccgattttaccctgaccattagccgcctggaaccggaagattttgcggtgtattattgccagcatcatagctggtatcattttacctttggcggcggcaccaaagtggaaattaaa

SEQ ID NO: 11 - protein, 113 aa, Homo sapiens
EVQLVESGGGLVQPGGSLRLSCAASGLTFSRYPMSWVRQAPGKGLVWVSAITSSGGSTYYSDTVKGRFTISRDNAKATLYLQMNSLRAEDTAVYYCARLPDYWGQGTLVTVSS

SEQ ID NO: 12 - protein, 108 aa, Homo sapiens
EIVLTQSPGTLSLSPGERATLSCRASSSVSSSYLHWYQQKPGQAPRLLIYSTSNLVAGIPDRFSGSGSGTDFTLTISRLEPEDFAVYYCQHHSGYHFTFGGGTKVEIK

SEQ ID NO: 13 - protein, 8 aa, Homo sapiens
GLTFSRYP

SEQ ID NO: 14 - protein, 8 aa, Homo sapiens
ITSSGGST

SEQ ID NO: 15 - protein, 6 aa, Homo sapiens
ARLPDY

SEQ ID NO: 16 - protein, 7 aa, Homo sapiens
SSVSSSY

SEQ ID NO: 17 - skipped

SEQ ID NO: 18 - protein, 9 aa, Homo sapiens
QHHSGYHFT

SEQ ID NO: 19 - DNA, 339 bp, Homo sapiens
  source 1..339: mol_type = unassigned DNA
gaagtgcagctggtggaaagcggcggcggcctggtgcagccgggcggcagcctgcgcctgagctgcgcggcgagcggcctgacctttagccgctatccgatgagctgggtgcgccaggcgccgggcaaaggcctggtgtgggtgagcgcgattaccagcagcggcggcagcacctattatagcgataccgtgaaaggccgctttaccattagccgcgataacgcgaaagcgaccctgtatctgcagatgaacagcctgcgcgcggaagataccgcggtgtattattgcgcgcgcctgccggattattggggccagggcaccctggtgaccgtgagcagc

SEQ ID NO: 20 - DNA, 324 bp, Homo sapiens
  source 1..324: mol_type = unassigned DNA
gaaattgtgctgacccagagcccgggcaccctgagcctgagcccgggcgaacgcgcgaccctgagctgccgcgcgagcagcagcgtgagcagcagctatctgcattggtatcagcagaaaccgggccaggcgccgcgcctgctgatttatagcaccagcaacctggtggcgggcattccggatcgctttagcggcagcggcagcggcaccgattttaccctgaccattagccgcctggaaccggaagattttgcggtgtattattgccagcatcatagcggctatcattttacctttggcggcggcaccaaagtggaaattaaa

SEQ ID NO: 21 - protein, 116 aa, Homo sapiens
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQVYAMNYWGQGTLVTVSS

SEQ ID NO: 22 - protein, 107 aa, Homo sapiens
DIQMTQSPSSLSASVGDRVTITCKASQDVSTAVAWYQQKPGKAPKLLIYTASYRETGVPSRFSGSGSGTDFTLTISSLQPEDFATYYCQQHYSTPWTFGGGTKVEIK

SEQ ID NO: 23 - protein, 8 aa, Homo sapiens
GFTFSSYA

SEQ ID NO: 24 - protein, 8 aa, Homo sapiens
ISSGGSYT

SEQ ID NO: 25 - protein, 9 aa, Homo sapiens
AKQVYAMNY

SEQ ID NO: 26 - protein, 6 aa, Homo sapiens
QDVSTA

SEQ ID NO: 27 - skipped

SEQ ID NO: 28 - protein, 9 aa, Homo sapiens
QQHYSTPWT

SEQ ID NO: 29 - DNA, 348 bp, Homo sapiens
  source 1..348: mol_type = unassigned DNA
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcaccattagcagcggcggcagctataccagctatccggatagcgtgaaaggccgctttaccattagccgcgataacagcaaaaacaccctgtatctgcagatgaacagcctgcgcgcggaagataccgcggtgtattattgcgcgaaacaggtgtatgcgatgaactattggggccagggcaccctggtgaccgtgagcagc

SEQ ID NO: 30 - DNA, 321 bp, Homo sapiens
  source 1..321: mol_type = unassigned DNA
gatattcagatgacccagagcccgagcagcctgagcgcgagcgtgggcgatcgcgtgaccattacctgcaaagcgagccaggatgtgagcaccgcggtggcgtggtatcagcagaaaccgggcaaagcgccgaaactgctgatttataccgcgagctatcgcgaaaccggcgtgccgagccgctttagcggcagcggcagcggcaccgattttaccctgaccattagcagcctgcagccggaagattttgcgacctattattgccagcagcattatagcaccccgtggacctttggcggcggcaccaaagtggaaattaaa

SEQ ID NO: 31 - protein, 116 aa, Homo sapiens
EVQLLESGGGLVQTGGSLRLSCAASGFTFSSYAMSWVRQAPGKGLEWVSTISSGGSYTSYPDSVKGRFTISRDNSKNTLYLQMNSLRAEDTAVYYCAKQDYAMNYWGQGTLVTVSS

SEQ ID NO: 32 - protein, 107 aa, Homo sapiens
DIQMTQSPSSLSASVGDRVTITCKSSQDVSTAVAWYQQKPGKAPKLLIYSASYRYTGVPSRFSGSGSGTDFTLSISSLQPEDFATYYCQQHYITPWTFGGGTKVEIK

SEQ ID NO: 33 - protein, 8 aa, Homo sapiens
GFTFSSYA

SEQ ID NO: 34 - protein, 8 aa, Homo sapiens
ISSGGSYT

SEQ ID NO: 35 - protein, 9 aa, Homo sapiens
AKQDYAMNY

SEQ ID NO: 36 - protein, 6 aa, Homo sapiens
QDVSTA

SEQ ID NO: 37 - skipped

SEQ ID NO: 38 - protein, 9 aa, Homo sapiens
QQHYITPWT

SEQ ID NO: 39 - DNA, 348 bp, Homo sapiens
  source 1..348: mol_type = unassigned DNA
gaagtgcagctgctggaaagcggcggcggcctggtgcagaccggcggcagcctgcgcctgagctgcgcggcgagcggctttacctttagcagctatgcgatgagctgggtgcgccaggcgccgggcaaaggcctggaatgggtgagcaccattagcagcggcggcagctataccagctatccggatagcgtgaaaggccgctttaccattagccgcgataacagcaaaaacaccctgtatctgcagatgaacagcctgcgcgcggaagataccgcggtgtattattgcgcgaaacaggattatgcgatgaactattggggccagggcaccctggtgaccgtgagcagc

SEQ ID NO: 40 - DNA, 321 bp, Homo sapiens
  source 1..321: mol_type = unassigned DNA
gatattcagatgacccagagcccgagcagcctgagcgcgagcgtgggcgatcgcgtgaccattacctgcaaaagcagccaggatgtgagcaccgcggtggcgtggtatcagcagaaaccgggcaaagcgccgaaactgctgatttatagcgcgagctatcgctataccggcgtgccgagccgctttagcggcagcggcagcggcaccgattttaccctgagcattagcagcctgcagccggaagattttgcgacctattattgccagcagcattatattaccccgtggacctttggcggcggcaccaaagtggaaattaaa

SEQ ID NO: 41 - protein, 113 aa, Homo sapiens; identical to SEQ ID NO: 1

SEQ ID NO: 42 - protein, 108 aa, Homo sapiens
EIVLTQSPGTLSLSPGERATLSCRASSSVSKSYLHWYQQKPGQAPRLLIYSTSNLVAGIPDRFSGSGSGTDFTLTISRLEPEDFAVYYCQHHSGPHGTFGGGTKVEIK

SEQ ID NO: 43 - protein, 8 aa, Homo sapiens
GLTFSRYP

SEQ ID NO: 44 - protein, 8 aa, Homo sapiens
ITSSGGST

SEQ ID NO: 45 - protein, 6 aa, Homo sapiens
ARLPDY

SEQ ID NO: 46 - protein, 7 aa, Homo sapiens
SSVSKSY

SEQ ID NO: 47 - skipped

SEQ ID NO: 48 - protein, 9 aa, Homo sapiens
QHHSGPHGT

SEQ ID NO: 49 - DNA, 339 bp, Homo sapiens; identical to SEQ ID NO: 9
  source 1..339: mol_type = unassigned DNA

SEQ ID NO: 50 - DNA, 324 bp, Homo sapiens
  source 1..324: mol_type = unassigned DNA
gaaattgtgctgacccagagcccgggcaccctgagcctgagcccgggcgaacgcgcgaccctgagctgccgcgcgagcagcagcgtgagcaaaagctatctgcattggtatcagcagaaaccgggccaggcgccgcgcctgctgatttatagcaccagcaacctggtggcgggcattccggatcgctttagcggcagcggcagcggcaccgattttaccctgaccattagccgcctggaaccggaagattttgcggtgtattattgccagcatcatagcggcccgcatggcacctttggcggcggcaccaaagtggaaattaaa

SEQ ID NO: 51 - protein, 113 aa, Homo sapiens
EVQLVESGGGLVQPGGSLRLSCTASGLTFSNYPMSWVRQAPGKGLVWVSAITSSGGSTYYSDTVKGRFTISRDNAKNTLYLQMNSLRAEDTAVYYCARLPDYWGQGTLVTVSS

SEQ ID NO: 52 - protein, 108 aa, Homo sapiens
EIVLTQSPGTLSLSPGERATLSCRASSSVSSSYLHWYQQKPGQAPRLLIYSTSNLVAGIPDRFSGSGSGTDFTLTISRLEPEDFAVYYCQHESGYHFTFGGGTKVEIK

SEQ ID NO: 53 - protein, 8 aa, Homo sapiens
GLTFSNYP

SEQ ID NO: 54 - protein, 8 aa, Homo sapiens
ITSSGGST

SEQ ID NO: 55 - protein, 6 aa, Homo sapiens
ARLPDY

SEQ ID NO: 56 - protein, 7 aa, Homo sapiens
SSVSSSY

SEQ ID NO: 57 - skipped

SEQ ID NO: 58 - protein, 9 aa, Homo sapiens
QHESGYHFT

SEQ ID NO: 59 - DNA, 339 bp, Homo sapiens
  source 1..339: mol_type = unassigned DNA
gaagtgcagctggtggaaagcggcggcggcctggtgcagccgggcggcagcctgcgcctgagctgcaccgcgagcggcctgacctttagcaactatccgatgagctgggtgcgccaggcgccgggcaaaggcctggtgtgggtgagcgcgattaccagcagcggcggcagcacctattatagcgataccgtgaaaggccgctttaccattagccgcgataacgcgaaaaacaccctgtatctgcagatgaacagcctgcgcgcggaagataccgcggtgtattattgcgcgcgcctgccggattattggggccagggcaccctggtgaccgtgagcagc

SEQ ID NO: 60 - DNA, 324 bp, Homo sapiens
  source 1..324: mol_type = unassigned DNA
gaaattgtgctgacccagagcccgggcaccctgagcctgagcccgggcgaacgcgcgaccctgagctgccgcgcgagcagcagcgtgagcagcagctatctgcattggtatcagcagaaaccgggccaggcgccgcgcctgctgatttatagcaccagcaacctggtggcgggcattccggatcgctttagcggcagcggcagcggcaccgattttaccctgaccattagccgcctggaaccggaagattttgcggtgtattattgccagcatgaaagcggctatcattttacctttggcggcggcaccaaagtggaaattaaa

SEQ ID NO: 61 - protein, 116 aa, Homo sapiens
EVQLLESGGGLVQPGGSLRLSCAASGFSFSSYAMSWVRQAPGKGLEWVSTISSGGSYTSYPDSVKGRFTISRDNSKNTLYLQMNSLRAEDTAVYYCAKQDYAMNYWGQGTLVTVSS

SEQ ID NO: 62 - protein, 107 aa, Homo sapiens
DIQMTQSPSSLSASVGDRVTITCKASQDVSTAVAWYQQKPGKAPKLLIYSASYRLTGVPSRFSGSGSGTDFTLTISSLQPEDFATYYCQQHYSTPWTFGGGTKVEIK

SEQ ID NO: 63 - protein, 8 aa, Homo sapiens
GFSFSSYA

SEQ ID NO: 64 - protein, 8 aa, Homo sapiens
ISSGGSYT

SEQ ID NO: 65 - protein, 9 aa, Homo sapiens
AKQDYAMNY

SEQ ID NO: 66 - protein, 6 aa, Homo sapiens
QDVSTA

SEQ ID NO: 67 - skipped

SEQ ID NO: 68 - protein, 9 aa, Homo sapiens
QQHYSTPWT

SEQ ID NO: 69 - DNA, 348 bp, Homo sapiens
  source 1..348: mol_type = unassigned DNA
gaagtgcagctgctggaaagcggcggcggcctggtgcagccgggcggcagcctgcgcctgagctgcgcggcgagcggctttagctttagcagctatgcgatgagctgggtgcgccaggcgccgggcaaaggcctggaatgggtgagcaccattagcagcggcggcagctataccagctatccggatagcgtgaaaggccgctttaccattagccgcgataacagcaaaaacaccctgtatctgcagatgaacagcctgcgcgcggaagataccgcggtgtattattgcgcgaaacaggattatgcgatgaactattggggccagggcaccctggtgaccgtgagcagc

SEQ ID NO: 70 - DNA, 321 bp, Homo sapiens
  source 1..321: mol_type = unassigned DNA
gatattcagatgacccagagcccgagcagcctgagcgcgagcgtgggcgatcgcgtgaccattacctgcaaagcgagccaggatgtgagcaccgcggtggcgtggtatcagcagaaaccgggcaaagcgccgaaactgctgatttatagcgcgagctatcgcctgaccggcgtgccgagccgctttagcggcagcggcagcggcaccgattttaccctgaccattagcagcctgcagccggaagattttgcgacctattattgccagcagcattatagcaccccgtggacctttggcggcggcaccaaagtggaaattaaa

SEQ ID NO: 71 - protein, 116 aa, Homo sapiens
EVQLLESGGGLVQPGGSLRLSCVASGFTFSSYAMSWVRQAPGKGLEWVSTISSGGSYTSYPDSVKGRFTISRDNSKNTLYLQMNSLRAEDTAVYYCAKQDYAMNYWGQGTLVTVSS

SEQ ID NO: 72 - protein, 107 aa, Homo sapiens
DIQMTQSPSSLSASVGDRVTITCKASQDVSTAVAWYQQKPGKAPKLLIYSASYRETGVPSRFSGSGSGTDFTLTISSLQPEDFATYYCQQHYSTPWTFGGGTKVEIK

SEQ ID NO: 73 - protein, 8 aa, Homo sapiens
GFTFSSYA

SEQ ID NO: 74 - protein, 8 aa, Homo sapiens
ISSGGSYT

SEQ ID NO: 75 - protein, 9 aa, Homo sapiens
AKQDYAMNY

SEQ ID NO: 76 - protein, 6 aa, Homo sapiens
QDVSTA

SEQ ID NO: 77 - skipped

SEQ ID NO: 78 - protein, 9 aa, Homo sapiens
QQHYSTPWT

SEQ ID NO: 79 - DNA, 348 bp, Homo sapiens
  source 1..348: mol_type = unassigned DNA
gaagtgcagctgctggaaagcggcggcggcctggtgcagccgggcggcagcctgcgcctgagctgcgtggcgagcggctttacctttagcagctatgcgatgagctgggtgcgccaggcgccgggcaaaggcctggaatgggtgagcaccattagcagcggcggcagctataccagctatccggatagcgtgaaaggccgctttaccattagccgcgataacagcaaaaacaccctgtatctgcagatgaacagcctgcgcgcggaagataccgcggtgtattattgcgcgaaacaggattatgcgatgaactattggggccagggcaccctggtgaccgtgagcagc

SEQ ID NO: 80 - DNA, 321 bp, Homo sapiens
  source 1..321: mol_type = unassigned DNA
gatattcagatgacccagagcccgagcagcctgagcgcgagcgtgggcgatcgcgtgaccattacctgcaaagcgagccaggatgtgagcaccgcggtggcgtggtatcagcagaaaccgggcaaagcgccgaaactgctgatttatagcgcgagctatcgcgaaaccggcgtgccgagccgctttagcggcagcggcagcggcaccgattttaccctgaccattagcagcctgcagccggaagattttgcgacctattattgccagcagcattatagcaccccgtggacctttggcggcggcaccaaagtggaaattaaa

SEQ ID NO: 81 - protein, 113 aa, Homo sapiens
EVQLVESGGGLVQPGGSLRLSCAASGLTFSRYPMSWVRQAPGKGLVWVSTITSSGGGTYYSDTVKGRFTISRDNAKNTLYLQMNSLRAEDTAVYYCARLPDYWGQGTLVTVSS

SEQ ID NO: 82 - protein, 108 aa, Homo sapiens
EIVLTQSPGTLSLSPGERATLSCRASSSVSSSYLHWYQQKPGQAPRLLIYSTSNLVAGIPDRFSGSGSGTDYTLTISRLEPEDFAVYYCQHHSGYPFTFGGGTKVEIK

SEQ ID NO: 83 - protein, 8 aa, Homo sapiens
GLTFSRYP

SEQ ID NO: 84 - protein, 8 aa, Homo sapiens
ITSSGGGT

SEQ ID NO: 85 - protein, 6 aa, Homo sapiens
ARLPDY

SEQ ID NO: 86 - protein, 7 aa, Homo sapiens
SSVSSSY

SEQ ID NO: 87 - skipped

SEQ ID NO: 88 - protein, 9 aa, Homo sapiens
QHHSGYPFT

SEQ ID NO: 89 - DNA, 339 bp, Homo sapiens
  source 1..339: mol_type = unassigned DNA
gaagtgcagctggtggaaagcggcggcggcctggtgcagccgggcggcagcctgcgcctgagctgcgcggcgagcggcctgacctttagccgctatccgatgagctgggtgcgccaggcgccgggcaaaggcctggtgtgggtgagcaccattaccagcagcggcggcggcacctattatagcgataccgtgaaaggccgctttaccattagccgcgataacgcgaaaaacaccctgtatctgcagatgaacagcctgcgcgcggaagataccgcggtgtattattgcgcgcgcctgccggattattggggccagggcaccctggtgaccgtgagcagc

SEQ ID NO: 90 - DNA, 324 bp, Homo sapiens
  source 1..324: mol_type = unassigned DNA
gaaattgtgctgacccagagcccgggcaccctgagcctgagcccgggcgaacgcgcgaccctgagctgccgcgcgagcagcagcgtgagcagcagctatctgcattggtatcagcagaaaccgggccaggcgccgcgcctgctgatttatagcaccagcaacctggtggcgggcattccggatcgctttagcggcagcggcagcggcaccgattataccctgaccattagccgcctggaaccggaagattttgcggtgtattattgccagcatcatagcggctatccgtttacctttggcggcggcaccaaagtggaaattaaa

SEQ ID NO: 91 - protein, 113 aa, Homo sapiens
EVQLVESGGGLVQPGGSLRLSCAASGLTFGRYPMSWVRQAPGKGLVWVSAITSSGGSTYYSDTVKGRFTISRDNAKTTLYLQMNSLRAEDTAIYYCARLPDYWGQGTLVTVSS

SEQ ID NO: 92 - protein, 108 aa, Homo sapiens
EIVLTQSPGTLSLSPGERVTLSCRASSSLSSSHLHWYQQKPGQAPRLVIYSTSNLVAGIPDRFSGSGSGTDFTLTISRLEPEDFAVYYCQHHSGYHFTFGGGTKVEIK

SEQ ID NO: 93 - protein, 8 aa, Homo sapiens
GLTFGRYP

SEQ ID NO: 94 - protein, 8 aa, Homo sapiens
ITSSGGST

SEQ ID NO: 95 - protein, 6 aa, Homo sapiens
ARLPDY

SEQ ID NO: 96 - protein, 7 aa, Homo sapiens
SSLSSSH

SEQ ID NO: 97 - skipped

SEQ ID NO: 98 - protein, 9 aa, Homo sapiens
QHHSGYHFT

SEQ ID NO: 99 - DNA, 339 bp, Homo sapiens
  source 1..339: mol_type = unassigned DNA
gaagtgcagctggtggaaagcggcggcggcctggtgcagccgggcggcagcctgcgcctgagctgcgcggcgagcggcctgacctttggccgctatccgatgagctgggtgcgccaggcgccgggcaaaggcctggtgtgggtgagcgcgattaccagcagcggcggcagcacctattatagcgataccgtgaaaggccgctttaccattagccgcgataacgcgaaaaccaccctgtatctgcagatgaacagcctgcgcgcggaagataccgcgatttattattgcgcgcgcctgccggattattggggccagggcaccctggtgaccgtgagcagc

SEQ ID NO: 100 - DNA, 324 bp, Homo sapiens
  source 1..324: mol_type = unassigned DNA
gaaattgtgctgacccagagcccgggcaccctgagcctgagcccgggcgaacgcgtgaccctgagctgccgcgcgagcagcagcctgagcagcagccatctgcattggtatcagcagaaaccgggccaggcgccgcgcctggtgatttatagcaccagcaacctggtggcgggcattccggatcgctttagcggcagcggcagcggcaccgattttaccctgaccattagccgcctggaaccggaagattttgcggtgtattattgccagcatcatagcggctatcattttacctttggcggcggcaccaaagtggaaattaaa

SEQ ID NO: 101 - protein, 116 aa, Homo sapiens
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDYAMDYWGQGTLVTVSS

SEQ ID NO: 102 - protein, 107 aa, Homo sapiens
DIQMTQSPSSLSASVGDRVTITCKASQDVSTAVAWYQQKPGKAPKLLMYSASYRYTGVPSRFSGSGSGTDFTLTIINLQPEDFATYYCQQHYSTPWTFGGGTKVEIK

SEQ ID NO: 103 - protein, 8 aa, Homo sapiens
GFTFSSYA

SEQ ID NO: 104 - protein, 8 aa, Homo sapiens
ISSGGSYT

SEQ ID NO: 105 - protein, 9 aa, Homo sapiens
AKQDYAMDY

SEQ ID NO: 106 - protein, 6 aa, Homo sapiens
QDVSTA

SEQ ID NO: 107 - skipped

SEQ ID NO: 108 - protein, 9 aa, Homo sapiens
QQHYSTPWT

SEQ ID NO: 109 - DNA, 348 bp, Homo sapiens
  source 1..348: mol_type = unassigned DNA
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcaccattagcagcggcggcagctataccagctatccggatagcgtgaaaggccgctttaccattagccgcgataacagcaaaaacaccctgtatctgcagatgaacagcctgcgcgcggaagataccgcggtgtattattgcgcgaaacaggattatgcgatggattattggggccagggcaccctggtgaccgtgagcagc

SEQ ID NO: 110 - DNA, 321 bp, Homo sapiens
  source 1..321: mol_type = unassigned DNA
gatattcagatgacccagagcccgagcagcctgagcgcgagcgtgggcgatcgcgtgaccattacctgcaaagcgagccaggatgtgagcaccgcggtggcgtggtatcagcagaaaccgggcaaagcgccgaaactgctgatgtatagcgcgagctatcgctataccggcgtgccgagccgctttagcggcagcggcagcggcaccgattttaccctgaccattattaacctgcagccggaagattttgcgacctattattgccagcagcattatagcaccccgtggacctttggcggcggcaccaaagtggaaattaaa

SEQ ID NO: 111 - protein, 113 aa, Homo sapiens; identical to SEQ ID NO: 1

SEQ ID NO: 112 - protein, 108 aa, Homo sapiens
EIVLTQSPGTLSLSPGERATLSCRASSSVSSSYLHWYQQKPGQAPRLLIYATSNLVAGIPDRFSGSGSGTDFTLTISRLEPEDFAVYYCQHFSGYHFTFGGGTKVEIK

SEQ ID NO: 113 - protein, 8 aa, Homo sapiens
GLTFSRYP

SEQ ID NO: 114 - protein, 8 aa, Homo sapiens
ITSSGGST

SEQ ID NO: 115 - protein, 6 aa, Homo sapiens
ARLPDY

SEQ ID NO: 116 - protein, 7 aa, Homo sapiens
SSVSSSY

SEQ ID NO: 117 - skipped

SEQ ID NO: 118 - protein, 9 aa, Homo sapiens
QHFSGYHFT

SEQ ID NO: 119 - DNA, 339 bp, Homo sapiens; identical to SEQ ID NO: 9
  source 1..339: mol_type = unassigned DNA

SEQ ID NO: 120 - DNA, 324 bp, Homo sapiens
  source 1..324: mol_type = unassigned DNA
gaaattgtgctgacccagagcccgggcaccctgagcctgagcccgggcgaacgcgcgaccctgagctgccgcgcgagcagcagcgtgagcagcagctatctgcattggtatcagcagaaaccgggccaggcgccgcgcctgctgatttatgcgaccagcaacctggtggcgggcattccggatcgctttagcggcagcggcagcggcaccgattttaccctgaccattagccgcctggaaccggaagattttgcggtgtattattgccagcattttagcggctatcattttacctttggcggcggcaccaaagtggaaattaaa

SEQ ID NO: 121 - protein, 113 aa, Homo sapiens
EVQLVESGGGLVQPGGSLRLSCAASGLTFSRYPMSWVRQAPGKGLVWVSAITSSGGSTYYSDTVKGRFTISRDNAKNTLYLQMNSLTAEDTAVYYCARLPDYWGQGTLVTVSS

SEQ ID NO: 122 - protein, 108 aa, Homo sapiens
EIVLTQSPGTLSLSPGERATLSCRASSSVSSSYLHWYQQKPGQAPRLLIYATSNLVAGIPDRFSGSGSGTDFTLTISRLEPEDFAVYYCQHHSGYHFTFGGGTKVEIK

SEQ ID NO: 123 - protein, 8 aa, Homo sapiens
GLTFSRYP

SEQ ID NO: 124 - protein, 8 aa, Homo sapiens
ITSSGGST

SEQ ID NO: 125 - protein, 6 aa, Homo sapiens
ARLPDY

SEQ ID NO: 126 - protein, 7 aa, Homo sapiens
SSVSSSY

SEQ ID NO: 127 - skipped

SEQ ID NO: 128 - protein, 9 aa, Homo sapiens
QHHSGYHFT

SEQ ID NO: 129 - DNA, 339 bp, Homo sapiens
  source 1..339: mol_type = unassigned DNA
gaagtgcagctggtggaaagcggcggcggcctggtgcagccgggcggcagcctgcgcctgagctgcgcggcgagcggcctgacctttagccgctatccgatgagctgggtgcgccaggcgccgggcaaaggcctggtgtgggtgagcgcgattaccagcagcggcggcagcacctattatagcgataccgtgaaaggccgctttaccattagccgcgataacgcgaaaaacaccctgtatctgcagatgaacagcctgaccgcggaagataccgcggtgtattattgcgcgcgcctgccggattattggggccagggcaccctggtgaccgtgagcagc

SEQ ID NO: 130 - DNA, 324 bp, Homo sapiens
  source 1..324: mol_type = unassigned DNA
gaaattgtgctgacccagagcccgggcaccctgagcctgagcccgggcgaacgcgcgaccctgagctgccgcgcgagcagcagcgtgagcagcagctatctgcattggtatcagcagaaaccgggccaggcgccgcgcctgctgatttatgcgaccagcaacctggtggcgggcattccggatcgctttagcggcagcggcagcggcaccgattttaccctgaccattagccgcctggaaccggaagattttgcggtgtattattgccagcatcatagcggctatcattttacctttggcggcggcaccaaagtggaaattaaa

SEQ ID NO: 131 - protein, 116 aa, Homo sapiens
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDYAMNQWGQGTLVTVSS

SEQ ID NO: 132 - protein, 107 aa, Homo sapiens
DIQMTQSPSSLSASVGDRVSITCKASQDVTTAVAWYQQKRGKAPKLLIYSASYRYTGVPSRFSGSGSGTDFTLTISSLQREDFATYYCQQHYSTPWTFGGGTKVEIK

SEQ ID NO: 133 - protein, 8 aa, Homo sapiens
GFTFSSYA

SEQ ID NO: 134 - protein, 8 aa, Homo sapiens
ISSGGSYT

SEQ ID NO: 135 - protein, 9 aa, Homo sapiens
AKQDYAMNQ

SEQ ID NO: 136 - protein, 6 aa, Homo sapiens
QDVTTA

SEQ ID NO: 137 - skipped

SEQ ID NO: 138 - protein, 9 aa, Homo sapiens
QQHYSTPWT

SEQ ID NO: 139 - DNA, 348 bp, Homo sapiens
  source 1..348: mol_type = unassigned DNA
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcaccattagcagcggcggcagctataccagctatccggatagcgtgaaaggccgctttaccattagccgcgataacagcaaaaacaccctgtatctgcagatgaacagcctgcgcgcggaagataccgcggtgtattattgcgcgaaacaggattatgcgatgaaccagtggggccagggcaccctggtgaccgtgagcagc

SEQ ID NO: 140 - DNA, 321 bp, Homo sapiens
  source 1..321: mol_type = unassigned DNA
gatattcagatgacccagagcccgagcagcctgagcgcgagcgtgggcgatcgcgtgagcattacctgcaaagcgagccaggatgtgaccaccgcggtggcgtggtatcagcagaaacgcggcaaagcgccgaaactgctgatttatagcgcgagctatcgctataccggcgtgccgagccgctttagcggcagcggcagcggcaccgattttaccctgaccattagcagcctgcagcgcgaagattttgcgacctattattgccagcagcattatagcaccccgtggacctttggcggcggcaccaaagtggaaattaaa

SEQ ID NO: 141 - protein, 113 aa, Homo sapiens; identical to SEQ ID NO: 1

SEQ ID NO: 142 - protein, 108 aa, Homo sapiens
EIVLTQSPGTLSLSPGERATLSCRASSSVSSSQLHWYQQKPGQAPRLLIYSTSNLVAGIPDRFSGSGSGTDFTLTISRLEPEDFAVYYCQHHSGSHFTFGGGTKVEIK

SEQ ID NO: 143 - protein, 8 aa, Homo sapiens
GLTFSRYP

SEQ ID NO: 144 - protein, 8 aa, Homo sapiens
ITSSGGST

SEQ ID NO: 145 - protein, 6 aa, Homo sapiens
ARLPDY

SEQ ID NO: 146 - protein, 7 aa, Homo sapiens
SSVSSSQ

SEQ ID NO: 147 - skipped

SEQ ID NO: 148 - protein, 9 aa, Homo sapiens
QHHSGSHFT

SEQ ID NO: 149 - DNA, 339 bp, Homo sapiens; identical to SEQ ID NO: 9
  source 1..339: mol_type = unassigned DNA

SEQ ID NO: 150 - DNA, 324 bp, Homo sapiens
  source 1..324: mol_type = unassigned DNA
gaaattgtgctgacccagagcccgggcaccctgagcctgagcccgggcgaacgcgcgaccctgagctgccgcgcgagcagcagcgtgagcagcagccagctgcattggtatcagcagaaaccgggccaggcgccgcgcctgctgatttatagcaccagcaacctggtggcgggcattccggatcgctttagcggcagcggcagcggcaccgattttaccctgaccattagccgcctggaaccggaagattttgcggtgtattattgccagcatcatagcggcagccattttacctttggcggcggcaccaaagtggaaattaaa

SEQ ID NO: 151 - protein, 113 aa, Homo sapiens
EVQLVESGGDLVQPGGSLRLSCATSGLTFSRYPMSWVRQAPGKGLVWVSAITSSGGSTYYSDTVKGRFTISRDNAKNTLYLQMNSLRAEDTAVYYCARLPDYWGQGTLVTVSS

SEQ ID NO: 152 - protein, 108 aa, Homo sapiens
EIVLTQSPGTLSLSPGERATLSCRASSSVSSSYLHWYQQKPGQAPRLLIYSTSNLVAGIPDRFSGSGSGTDFTLTISRLEPEDFAVYYCQHSSGYHITFGGGTKVEIK

SEQ ID NO: 153 - protein, 8 aa, Homo sapiens
GLTFSRYP

SEQ ID NO: 154 - protein, 8 aa, Homo sapiens
ITSSGGST

SEQ ID NO: 155 - protein, 6 aa, Homo sapiens
ARLPDY

SEQ ID NO: 156 - protein, 7 aa, Homo sapiens
SSVSSSY

SEQ ID NO: 157 - skipped

SEQ ID NO: 158 - protein, 9 aa, Homo sapiens
QHSSGYHIT

SEQ ID NO: 159 - DNA, 339 bp, Homo sapiens
  source 1..339: mol_type = unassigned DNA
gaagtgcagctggtggaaagcggcggcgatctggtgcagccgggcggcagcctgcgcctgagctgcgcgaccagcggcctgacctttagccgctatccgatgagctgggtgcgccaggcgccgggcaaaggcctggtgtgggtgagcgcgattaccagcagcggcggcagcacctattatagcgataccgtgaaaggccgctttaccattagccgcgataacgcgaaaaacaccctgtatctgcagatgaacagcctgcgcgcggaagataccgcggtgtattattgcgcgcgcctgccggattattggggccagggcaccctggtgaccgtgagcagc

SEQ ID NO: 160 - DNA, 324 bp, Homo sapiens
  source 1..324: mol_type = unassigned DNA
gaaattgtgctgacccagagcccgggcaccctgagcctgagcccgggcgaacgcgcgaccctgagctgccgcgcgagcagcagcgtgagcagcagctatctgcattggtatcagcagaaaccgggccaggcgccgcgcctgctgatttatagcaccagcaacctggtggcgggcattccggatcgctttagcggcagcggcagcggcaccgattttaccctgaccattagccgcctggaaccggaagattttgcggtgtattattgccagcatagcagcggctatcatattacctttggcggcggcaccaaagtggaaattaaa

SEQ ID NO: 161 - protein, 116 aa, Homo sapiens
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDYAMNYWGQGTLVTVSS

SEQ ID NO: 162 - protein, 107 aa, Homo sapiens
DIQMTQSPSSLSASVGDRVTITCKASQDVSTAVAWYQQKPGKAPKLLIYSASYRYTGVPSRFSGSGSGTDFTLTISSLLPEDFATYYCQQHYSTPWTFGGGTKVEIK

SEQ ID NO: 163 - protein, 8 aa, Homo sapiens
GFTFSSYA

SEQ ID NO: 164 - protein, 8 aa, Homo sapiens
ISSGGSYT

SEQ ID NO: 165 - protein, 9 aa, Homo sapiens
AKQDYAMNY

SEQ ID NO: 166 - protein, 6 aa, Homo sapiens
QDVSTA

SEQ ID NO: 167 - skipped

SEQ ID NO: 168 - protein, 9 aa, Homo sapiens
QQHYSTPWT

SEQ ID NO: 169 - DNA, 348 bp, Homo sapiens
  source 1..348: mol_type = unassigned DNA
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcaccattagcagcggcggcagctataccagctatccggatagcgtgaaaggccgctttaccattagccgcgataacagcaaaaacaccctgtatctgcagatgaacagcctgcgcgcggaagataccgcggtgtattattgcgcgaaacaggattatgcgatgaactattggggccagggcaccctggtgaccgtgagcagc

SEQ ID NO: 170 - DNA, 321 bp, Homo sapiens
  source 1..321: mol_type = unassigned DNA
gatattcagatgacccagagcccgagcagcctgagcgcgagcgtgggcgatcgcgtgaccattacctgcaaagcgagccaggatgtgagcaccgcggtggcgtggtatcagcagaaaccgggcaaagcgccgaaactgctgatttatagcgcgagctatcgctataccggcgtgccgagccgctttagcggcagcggcagcggcaccgattttaccctgaccattagcagcctgctgccggaagattttgcgacctattattgccagcagcattatagcaccccgtggacctttggcggcggcaccaaagtggaaattaaa

SEQ ID NO: 171 - protein, 113 aa, Homo sapiens
EVQLVESGGGLVQPGGSLRLSCAASGFTVSSYGMSWVRQAPGKGLEWVSGIRGSDGSTYYADSVKGRFTISRDNSKNTLYLQMNSLRAEDTAVYYCARLPDYWGQGTLVTVSS

SEQ ID NO: 172 - protein, 108 aa, Homo sapiens
EIVLTQSPGTQSLSPGERATLSCRASSAVSGSYLHWYQQKPGQAPRLLIYSTSNLVAGIPDRFSGSGSGTDFTLTITRLEPEDFAVYYCQHHSDYHYTFGGGTKVEIK

SEQ ID NO: 173 - protein, 8 aa, Homo sapiens
GFTVSSYG

SEQ ID NO: 174 - protein, 8 aa, Homo sapiens
IRGSDGST

SEQ ID NO: 175 - protein, 6 aa, Homo sapiens
ARLPDY

SEQ ID NO: 176 - protein, 7 aa, Homo sapiens
SAVSGSY

SEQ ID NO: 177 - skipped

SEQ ID NO: 178 - protein, 9 aa, Homo sapiens
QHHSDYHYT

SEQ ID NO: 179 - DNA, 339 bp, Homo sapiens
  source 1..339: mol_type = unassigned DNA
gaagtgcagctggtggaaagcggcggcggcctggtgcagccgggcggcagcctgcgcctgagctgcgcggcgagcggctttaccgtgagcagctatggcatgagctgggtgcgccaggcgccgggcaaaggcctggaatgggtgagcggcattcgcggcagcgatggcagcacctattatgcggatagcgtgaaaggccgctttaccattagccgcgataacagcaaaaacaccctgtatctgcagatgaacagcctgcgcgcggaagataccgcggtgtattattgcgcgcgcctgccggattattggggccagggcaccctggtgaccgtgagcagc

SEQ ID NO: 180 - DNA, 324 bp, Homo sapiens
  source 1..324: mol_type = unassigned DNA
gaaattgtgctgacccagagcccgggcacccagagcctgagcccgggcgaacgcgcgaccctgagctgccgcgcgagcagcgcggtgagcggcagctatctgcattggtatcagcagaaaccgggccaggcgccgcgcctgctgatttatagcaccagcaacctggtggcgggcattccggatcgctttagcggcagcggcagcggcaccgattttaccctgaccattacccgcctggaaccggaagattttgcggtgtattattgccagcatcatagcgattatcattatacctttggcggcggcaccaaagtggaaattaaa

SEQ ID NO: 181 - protein, 113 aa, Homo sapiens
EVQLVESGGGLVQPGGSLRLSCAASGLTFSRYPMSWVRQAPGKGLVWVSSITSSGGSTDYSDTVKGRFTISRDNAKNTLYLQMNSLRAEDTAVYYCARLPDYWGQGTLVTVSS

SEQ ID NO: 182 - protein, 108 aa, Homo sapiens
EIVLTQSPGILSLSPGERVTLSCRASSSVSSSYLHWYQQKPGQAPRLLIYSTSNLVAGIPDRFSGSGSGTEFTLTISRLEPEDFAVYYCQHHSTPHFTFGGGTKVEIK

SEQ ID NO: 183 - protein, 8 aa, Homo sapiens
GLTFSRYP

SEQ ID NO: 184 - protein, 8 aa, Homo sapiens
ITSSGGST

SEQ ID NO: 185 - protein, 6 aa, Homo sapiens
ARLPDY

SEQ ID NO: 186 - protein, 7 aa, Homo sapiens
SSVSSSY

SEQ ID NO: 187 - skipped

SEQ ID NO: 188 - protein, 9 aa, Homo sapiens
QHHSTPHFT

SEQ ID NO: 189 - DNA, 339 bp, Homo sapiens
  source 1..339: mol_type = unassigned DNA
gaagtgcagctggtggaaagcggcggcggcctggtgcagccgggcggcagcctgcgcctgagctgcgcggcgagcggcctgacctttagccgctatccgatgagctgggtgcgccaggcgccgggcaaaggcctggtgtgggtgagcagcattaccagcagcggcggcagcaccgattatagcgataccgtgaaaggccgctttaccattagccgcgataacgcgaaaaacaccctgtatctgcagatgaacagcctgcgcgcggaagataccgcggtgtattattgcgcgcgcctgccggattattggggccagggcaccctggtgaccgtgagcagc

SEQ ID NO: 190 - DNA, 324 bp, Homo sapiens
  source 1..324: mol_type = unassigned DNA
gaaattgtgctgacccagagcccgggcattctgagcctgagcccgggcgaacgcgtgaccctgagctgccgcgcgagcagcagcgtgagcagcagctatctgcattggtatcagcagaaaccgggccaggcgccgcgcctgctgatttatagcaccagcaacctggtggcgggcattccggatcgctttagcggcagcggcagcggcaccgaatttaccctgaccattagccgcctggaaccggaagattttgcggtgtattattgccagcatcatagcaccccgcattttacctttggcggcggcaccaaagtggaaattaaa

SEQ ID NO: 191 - protein, 116 aa, Homo sapiens
EVQLLESGGGLVQPGGSLRLSCAASGFTFSSNAMSWVRQAPGKGLEWVSTISSGGSYTSYPDSVKGRFTISRDNSKNTLYLQMNSLRAEDTAVYYCAKQDYAMNYWGQGTLVTVSS

SEQ ID NO: 192 - protein, 107 aa, Homo sapiens
DIQMTQSPSSLSASVGDRVTITCKASQDVSDAVAWYQQTPGKAPKLLIYSASYRPTGVPSRFSGSGSGTDFTLTISSLQPEDFATYYCQQHYSTPWTFGGGTKVEIK

SEQ ID NO: 193 - protein, 8 aa, Homo sapiens
GFTFSSNA

SEQ ID NO: 194 - protein, 8 aa, Homo sapiens
ISSGGSYT

SEQ ID NO: 195 - protein, 9 aa, Homo sapiens
AKQDYAMNY

SEQ ID NO: 196 - protein, 6 aa, Homo sapiens
QDVSDA

SEQ ID NO: 197 - skipped

SEQ ID NO: 198 - protein, 9 aa, Homo sapiens
QQHYSTPWT

SEQ ID NO: 199 - DNA, 348 bp, Homo sapiens
  source 1..348: mol_type = unassigned DNA
gaagtgcagctgctggaaagcggcggcggcctggtgcagccgggcggcagcctgcgcctgagctgcgcggcgagcggctttacctttagcagcaacgcgatgagctgggtgcgccaggcgccgggcaaaggcctggaatgggtgagcaccattagcagcggcggcagctataccagctatccggatagcgtgaaaggccgctttaccattagccgcgataacagcaaaaacaccctgtatctgcagatgaacagcctgcgcgcggaagataccgcggtgtattattgcgcgaaacaggattatgcgatgaactattggggccagggcaccctggtgaccgtgagcagc

SEQ ID NO: 200 - DNA, 321 bp, Homo sapiens
  source 1..321: mol_type = unassigned DNA
gatattcagatgacccagagcccgagcagcctgagcgcgagcgtgggcgatcgcgtgaccattacctgcaaagcgagccaggatgtgagcgatgcggtggcgtggtatcagcagaccccgggcaaagcgccgaaactgctgatttatagcgcgagctatcgcccgaccggcgtgccgagccgctttagcggcagcggcagcggcaccgattttaccctgaccattagcagcctgcagccggaagattttgcgacctattattgccagcagcattatagcaccccgtggacctttggcggcggcaccaaagtggaaattaaa

SEQ ID NO: 201 - protein, 113 aa, Homo sapiens
EVQLVESGGGLVQPGGSLRLSCAASGLTFSRYPMSWVRQAPGKGLVWVSAITSSGGSTYYSDTVKGRFTISRDNAKNTLYLQMNSLRAEDTAIYYCVRLPDYWGQGTLVTVSS

SEQ ID NO: 202 - protein, 108 aa, Homo sapiens
EIVLTQSPGTLSLSPGERATLSCRASSIVSSSYLHWYQQKPGQAPRLLIYSTFNLVADIPDRFSGSGSGTDFTLTISRLEPEDFAVYYCQHHSGYPFTFGGGTKVEIK

SEQ ID NO: 203 - protein, 8 aa, Homo sapiens
GLTFSRYP

SEQ ID NO: 204 - protein, 8 aa, Homo sapiens
ITSSGGST

SEQ ID NO: 205 - protein, 6 aa, Homo sapiens
VRLPDY

SEQ ID NO: 206 - protein, 7 aa, Homo sapiens
SIVSSSY

SEQ ID NO: 207 - skipped

SEQ ID NO: 208 - protein, 9 aa, Homo sapiens
QHHSGYPFT

SEQ ID NO: 209 - DNA, 339 bp, Homo sapiens
  source 1..339: mol_type = unassigned DNA
gaagtgcagctggtggaaagcggcggcggcctggtgcagccgggcggcagcctgcgcctgagctgcgcggcgagcggcctgacctttagccgctatccgatgagctgggtgcgccaggcgccgggcaaaggcctggtgtgggtgagcgcgattaccagcagcggcggcagcacctattatagcgataccgtgaaaggccgctttaccattagccgcgataacgcgaaaaacaccctgtatctgcagatgaacagcctgcgcgcggaagataccgcgatttattattgcgtgcgcctgccggattattggggccagggcaccctggtgaccgtgagcagc

SEQ ID NO: 210 - DNA, 324 bp, Homo sapiens
  source 1..324: mol_type = unassigned DNA
gaaattgtgctgacccagagcccgggcaccctgagcctgagcccgggcgaacgcgcgaccctgagctgccgcgcgagcagcattgtgagcagcagctatctgcattggtatcagcagaaaccgggccaggcgccgcgcctgctgatttatagcacctttaacctggtggcggatattccggatcgctttagcggcagcggcagcggcaccgattttaccctgaccattagccgcctggaaccggaagattttgcggtgtattattgccagcatcatagcggctatccgtttacctttggcggcggcaccaaagtggaaattaaa

SEQ ID NO: 211 - protein, 116 aa, Homo sapiens; identical to SEQ ID NO: 161

SEQ ID NO: 212 - protein, 107 aa, Homo sapiens
DIQMTQSPSSLSASVGDRVTITCKASQDVSTAVAWYQQKPGKAPKLLIYSASYRYTGVPSRFSGSGSGTDFTLTISSLQPEDFATYHCQQHYSTPWTFGGGTKVEIK

SEQ ID NO: 213 - protein, 8 aa, Homo sapiens
GFTFSSYA

SEQ ID NO: 214 - protein, 8 aa, Homo sapiens
ISSGGSYT

SEQ ID NO: 215 - protein, 9 aa, Homo sapiens
AKQDYAMNY

SEQ ID NO: 216 - protein, 6 aa, Homo sapiens
QDVSTA

SEQ ID NO: 217 - skipped

SEQ ID NO: 218 - protein, 9 aa, Homo sapiens
QQHYSTPWT

SEQ ID NO: 219 - DNA, 348 bp, Homo sapiens; identical to SEQ ID NO: 169
  source 1..348: mol_type = unassigned DNA

SEQ ID NO: 220 - DNA, 321 bp, Homo sapiens
  source 1..321: mol_type = unassigned DNA
gatattcagatgacccagagcccgagcagcctgagcgcgagcgtgggcgatcgcgtgaccattacctgcaaagcgagccaggatgtgagcaccgcggtggcgtggtatcagcagaaaccgggcaaagcgccgaaactgctgatttatagcgcgagctatcgctataccggcgtgccgagccgctttagcggcagcggcagcggcaccgattttaccctgaccattagcagcctgcagccggaagattttgcgacctatcattgccagcagcattatagcaccccgtggacctttggcggcggcaccaaagtggaaattaaa

SEQ ID NO: 221 - protein, 116 aa, Homo sapiens
EVQLLESGGGLVQPGGSLRLSCAASGFTFSSYAMSWVRQAPGKGLEWVSTISSGGSYTSYPDSVKGRFTISRDNSKNTLYLQMNSLRGEDTALYYCARQDYAMNYWGQGTLVTVSS

SEQ ID NO: 222 - protein, 107 aa, Homo sapiens
DIQMTQSPSSLSASVGDRVTITCKASQDVSTAVAWYQQKPGKAPNLLIYSASYRYTGVPSRFSGSGSGTDFTLTISSLQPEDFATYYCQQHYSTPWTFGGGTKVEIK

SEQ ID NO: 223 - protein, 8 aa, Homo sapiens
GFTFSSYA

SEQ ID NO: 224 - protein, 8 aa, Homo sapiens
ISSGGSYT

SEQ ID NO: 225 - protein, 9 aa, Homo sapiens
ARQDYAMNY

SEQ ID NO: 226 - protein, 6 aa, Homo sapiens
QDVSTA

SEQ ID NO: 227 - skipped

SEQ ID NO: 228 - protein, 9 aa, Homo sapiens
QQHYSTPWT

SEQ ID NO: 229 - DNA, 348 bp, Homo sapiens
  source 1..348: mol_type = unassigned DNA
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcaccattagcagcggcggcagctataccagctatccggatagcgtgaaaggccgctttaccattagccgcgataacagcaaaaacaccctgtatctgcagatgaacagcctgcgcggcgaagataccgcgctgtattattgcgcgcgccaggattatgcgatgaactattggggccagggcaccctggtgaccgtgagcagc

SEQ ID NO: 230 - DNA, 321 bp, Homo sapiens
  source 1..321: mol_type = unassigned DNA
gatattcagatgacccagagcccgagcagcctgagcgcgagcgtgggcgatcgcgtgaccattacctgcaaagcgagccaggatgtgagcaccgcggtggcgtggtatcagcagaaaccgggcaaagcgccgaacctgctgatttatagcgcgagctatcgctataccggcgtgccgagccgctttagcggcagcggcagcggcaccgattttaccctgaccattagcagcctgcagccggaagattttgcgacctattattgccagcagcattatagcaccccgtggacctttggcggcggcaccaaagtggaaattaaa

SEQ ID NO: 231 - protein, 113 aa, Homo sapiens
EVQLVESGGGLVQPGGSLRLSCAASGLTFSRYPMSWVRQAPGKGLVWVSAITSSGGSTYYSDTVKGRFTISRDNAKNTLFLQMNSLRAEDTAVYYCARLPDYWGQGTLVTVSS

SEQ ID NO: 232 - protein, 108 aa, Homo sapiens
EIVLTQSPGTLSLSPGERATLSCRASSSVSISYLHWYQVKPGQAPRLLIYSTSNLVAGIPDRFSGSGSGTDFTLTISRLEPEDFAVYFCQHESGYHFTFGGGTKVEIK

SEQ ID NO: 233 - protein, 8 aa, Homo sapiens
GLTFSRYP

SEQ ID NO: 234 - protein, 8 aa, Homo sapiens
ITSSGGST

SEQ ID NO: 235 - protein, 6 aa, Homo sapiens
ARLPDY

SEQ ID NO: 236 - protein, 7 aa, Homo sapiens
SSVSISY

SEQ ID NO: 237 - skipped

SEQ ID NO: 238 - protein, 9 aa, Homo sapiens
QHESGYHFT

SEQ ID NO: 239 - DNA, 339 bp, Homo sapiens
  source 1..339: mol_type = unassigned DNA
gaagtgcagctggtggaaagcggcggcggcctggtgcagccgggcggcagcctgcgcctgagctgcgcggcgagcggcctgacctttagccgctatccgatgagctgggtgcgccaggcgccgggcaaaggcctggtgtgggtgagcgcgattaccagcagcggcggcagcacctattatagcgataccgtgaaaggccgctttaccattagccgcgataacgcgaaaaacaccctgtttctgcagatgaacagcctgcgcgcggaagataccgcggtgtattattgcgcgcgcctgccggattattggggccagggcaccctggtgaccgtgagcagc

SEQ ID NO: 240 - DNA, 324 bp, Homo sapiens
  source 1..324: mol_type = unassigned DNA
gaaattgtgctgacccagagcccgggcaccctgagcctgagcccgggcgaacgcgcgaccctgagctgccgcgcgagcagcagcgtgagcattagctatctgcattggtatcaggtgaaaccgggccaggcgccgcgcctgctgatttatagcaccagcaacctggtggcgggcattccggatcgctttagcggcagcggcagcggcaccgattttaccctgaccattagccgcctggaaccggaagattttgcggtgtatttttgccagcatgaaagcggctatcattttacctttggcggcggcaccaaagtggaaattaaa

SEQ ID NO: 241 - protein, 116 aa, Homo sapiens
EVQLLESGGGLVQPGRSLRLSCAASGFTFHSYAMSWVRQAPGKGLEWVSTISSGGSYTSYPDSVKGRFTISRDNSKNTLYLQMNSLRAEDTAVYYCAKQDYAMDLWGQGTLVTVSS

SEQ ID NO: 242 - protein, 107 aa, Homo sapiens
DIQMTQSPSSLSASVGDRVTITCKASQDVSTAVAWYQQKPGKAPKLLMYSASYRRTGVPSRFSGSGSGTDFTLTISSLQPEDVATYYCQQHYSTPWTFGGGTKVEIK

SEQ ID NO: 243 - protein, 8 aa, Homo sapiens
GFTFHSYA

SEQ ID NO: 244 - protein, 8 aa, Homo sapiens
ISSGGSYT

SEQ ID NO: 245 - protein, 9 aa, Homo sapiens
AKQDYAMDL

SEQ ID NO: 246 - protein, 6 aa, Homo sapiens
SQDVST

SEQ ID NO: 247 - skipped

SEQ ID NO: 248 - protein, 10 aa, Homo sapiens
CQQHYSTPWT

SEQ ID NO: 249 - DNA, 348 bp, Homo sapiens
  source 1..348: mol_type = unassigned DNA
gaagtgcagctgctggaaagcggcggcggcctggtgcagccgggccgcagcctgcgcctgagctgcgcggcgagcggctttacctttcatagctatgcgatgagctgggtgcgccaggcgccgggcaaaggcctggaatgggtgagcaccattagcagcggcggcagctataccagctatccggatagcgtgaaaggccgctttaccattagccgcgataacagcaaaaacaccctgtatctgcagatgaacagcctgcgcgcggaagataccgcggtgtattattgcgcgaaacaggattatgcgatggatctgtggggccagggcaccctggtgaccgtgagcagc

SEQ ID NO: 250 - DNA, 321 bp, Homo sapiens
  source 1..321: mol_type = unassigned DNA
gatattcagatgacccagagcccgagcagcctgagcgcgagcgtgggcgatcgcgtgaccattacctgcaaagcgagccaggatgtgagcaccgcggtggcgtggtatcagcagaaaccgggcaaagcgccgaaactgctgatgtatagcgcgagctatcgccgcaccggcgtgccgagccgctttagcggcagcggcagcggcaccgattttaccctgaccattagcagcctgcagccggaagatgtggcgacctattattgccagcagcattatagcaccccgtggacctttggcggcggcaccaaagtggaaattaaa

SEQ ID NO: 251 - protein, 115 aa, Homo sapiens
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDAMNYWGQGTLVTVSS

SEQ ID NO: 252 - protein, 107 aa, Homo sapiens
DIQMTQSPSSLSASVGDRVTITCKASQDVSTAVAWYQQKPGKAPKLLIYSASYRYTGVPSRFSGSGSGTDFTLTISRLQPEDFATYYCQQHYSTPWTFGGGTKVEIK

SEQ ID NO: 253 - protein, 8 aa, Homo sapiens
GFTFSSYA

SEQ ID NO: 254 - protein, 8 aa, Homo sapiens
ISSGGSYT

SEQ ID NO: 255 - protein, 9 aa, Homo sapiens
AKQDYAMNY

SEQ ID NO: 256 - protein, 6 aa, Homo sapiens
QDVSTA

SEQ ID NO: 257 - skipped

SEQ ID NO: 258 - protein, 9 aa, Homo sapiens
QQHYSTPWT

SEQ ID NO: 259 - DNA, 345 bp, Homo sapiens
  source 1..345: mol_type = unassigned DNA
gaagtgcagctgctggaaagcggcggcggcctggtgcagccgggcggcagcctgcgcctgagctgcgcggcgagcggctttacctttagcagctatgcgatgagctgggtgcgccaggcgccgggcaaaggcctggaatgggtgagcaccattagcagcggcggcagctataccagctatccggatagcgtgaaaggccgctttaccattagccgcgataacagcaaaaacaccctgtatctgcagatgaacagcctgcgcgcggaagataccgcggtgtattattgcgcgaaacaggatgcgatgaactattggggccagggcaccctggtgaccgtgagcagc

SEQ ID NO: 260 - DNA, 321 bp, Homo sapiens
  source 1..321: mol_type = unassigned DNA
gatattcagatgacccagagcccgagcagcctgagcgcgagcgtgggcgatcgcgtgaccattacctgcaaagcgagccaggatgtgagcaccgcggtggcgtggtatcagcagaaaccgggcaaagcgccgaaactgctgatttatagcgcgagctatcgctataccggcgtgccgagccgctttagcggcagcggcagcggcaccgattttaccctgaccattagccgcctgcagccggaagattttgcgacctattattgccagcagcattatagcaccccgtggacctttggcggcggcaccaaagtggaaattaaa

SEQ ID NO: 261 - protein, 330 aa, Homo sapiens [sequence omitted]

SEQ ID NO: 262 - DNA, 990 bp, Homo sapiens
  source 1..990: mol_type = unassigned DNA
gcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 263 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 264 - DNA, 978 bp, Homo sapiens
  source 1..978: mol_type = unassigned DNA
gccagtacaaaaggacctagcgtgttccctttggctccctgtagtcgttcaacctctgaaagcacagcagcgctggggtgtttggtcaaggactattttcccgaacccgtgaccgtcagctggaattctggggcgttgacctctggagttcatacatttcccgctgtgctccaaagttccggcttgtattcccttagtagcgttgttaccgttccatcatccagcttgggcaccaaaacttatacctgtaatgttgatcataaaccctcaaatacaaaggtcgataagagggtcgaaagcaaatacgggcccccttgtccgccctgtccagcgcccgagtttctgggcgggccttctgtgttcttgttcccgccaaaacccaaagatactctcatgattagccgaactcccgaggtgacatgcgtcgtcgttgatgtctctcaagaagatcctgaagtgcaatttaattggtatgttgatggcgtggaagtacacaatgctaagaccaagccccgcgaggaacaatttaatagcacttaccgtgtggtgagcgttctcactgtcctgcatcaagactggttgaatggtaaagaatataaatgcaaagtgtccaataaaggactgcccagcagcatcgaaaagaccatatccaaagctaaggggcaaccgcgagaacctcaagtttatacgctcccaccatcccaagaagaaatgacaaagaaccaagtgtcccttacttgcttggtgaagggcttttacccttcagatatagcagtagaatgggaaagcaatggccaacctgaaaacaattataaaacaactcccccagtactggatagtgatggctctttcttcctttatagccgtcttacagttgacaagagccgctggcaagaaggcaatgtgttctcttgttcagtcatgcacgaagcgctccacaatcattatactcagaagagtttgtcactcagtttgggg

SEQ ID NO: 265 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 266 - DNA, 978 bp, Homo sapiens
  source 1..978: mol_type = unassigned DNA
gccagtacaaaaggacctagcgtgttccctttggctccctgtagtcgttcaacctctgaaagcacagcagcgctggggtgtttggtcaaggactattttcccgaacccgtgaccgtcagctggaattctggggcgttgacctctggagttcatacatttcccgctgtgctccaaagttccggcttgtattcccttagtagcgttgttaccgttccatcatccagcttgggcaccaaaacttatacctgtaatgttgatcataaaccctcaaatacaaaggtcgataagagggtcgaaagcaaatacgggcccccttgtccgccctgtccagcgcccgagtttctgggcgggccttctgtgttcttgttcccgccaaaacccaaagatactctcatgattagccgaactcccgaggtgacatgcgtcgtcgttgatgtctctcaagaagatcctgaagtgcaatttaattggtatgttgatggcgtggaagtacacaatgctaagaccaagccccgcgaggaacaatttaatagcacttaccgtgtggtgagcgttctcactgtcctgcatcaagactggttgaatggtaaagaatataaatgcaaagtgtccaataaaggactgcccagcagcatcgaaaagaccatatccaaagctaaggggcaaccgcgagaacctcaagtttatacgctcccaccatcccaagaagaaatgacaaagaaccaagtgtcccttacttgcttggtgaagggcttttacccttcagatatagcagtagaatgggaaagcaatggccaacctgaaaacaattataaaacaactcccccagtactggatagtgatggctctttcttcctttatagccgtcttacagttgacaagagccgctggcaagaaggcaatgtgttctcttgttcagtcctgcacgaagcgctccactctcattatactcagaagagtttgtcactcagtttgggg

SEQ ID NO: 267 - protein, 15 aa, synthetic construct
GGGGSGGGGSGGGGS